(19) United States
(12) Patent Application Publication
(10) Pub. No.: US 2005/0208054 A1
(43) Pub. Date: Sep. 22, 2005

(54) Methods of identifying insulin response modulators and uses therefor

(75) Inventors: Michael P. Czech, Westborough, MA (US); Zhen Y. Jiang, Shrewsbury, MA (US)

Correspondence Address: LAHIVE & COCKFIELD, LLP., 28 STATE STREET, BOSTON, MA, 02109, US

(73) Assignee: UNIVERSITY OF MASSACHUSETTS, Boston, MA (US)

(21) Appl. No.: 11/009,554

(22) Filed: Dec. 9, 2004

Related U.S. Application Data
(60) Provisional application No. 60/528,354, filed on Dec. 9, 2003.

(51) Int. Cl. G01N 33/53; G01N 33/567; A61K 39/395

(52) U.S. Cl. 424/146.1; 435/7.2

(57) ABSTRACT

Methods of identifying insulin response modulators are provided. In particular, methods that feature identifying modulators of Akt and its associated substrates (including R-type calcium channel alpha-1E subunit (R-CaC1E), WNK1, FMS interacting protein (FMIP), nGAP-like protein, nuclear matrix protein p84, HIRA interacting protein 3 (HIRIP3), HSP71, ribosomal protein L6, guanine nucleotide exchange factor Lbc (GEF Lbc), ATP citrate lyase, Mi-2b, peripheral benzodiazepine receptor-associated protein 1, heterogeneous nuclear ribonucleoprotein U (hnRNP U protein), pyruvate carboxylase precursor, Eps domain containing protein (RalBP1), nonmuscle myosin IIA (NMMIIA), and stress 70 protein (p66 mot1/GRP75), or activities associated therewith, are provided. Therapeutic methods utilizing compounds identified according to the methods of the invention are also provided.

### RELATED APPLICATIONS

This application claims priority to U.S. Provisional Application No. 60/528,354 filed on Dec. 9, 2003, the contents of which are incorporated herein by reference.

### GOVERNMENT RIGHTS

This invention was made at least in part with government support under Grant No. DK30648 awarded by the National Institutes of Health. The government may have certain rights in this invention.

### BACKGROUND OF THE INVENTION

The regulation of blood glucose levels by insulin is achieved mainly by increased glucose transport exclusively into adipose and skeletal muscle tissue; De Fronzo et al. (1981) Diabetes 30:1000-1007 and James et al. (1985) Am. J. Physiol. 248:E567-E574. These are the only two tissues that express a specific isoform of the glucose transporter, GLUT4, which mediates the hormonal effect of insulin (for reviews of glucose transporter isoforms and their expression, see Deveskar and Mueckler (1992) Pediatr. Res. 31:1-13; Bell et al. (1993) J. Biol. Chem. 268:3352-3356; and Baldwin (1993) Biochim. Biophys. Acta 1154:17-49). The mechanism of glucose transport activation by insulin is the hormone-dependent enhancement of the rate of GLUT4 translocation from intracellular storage vesicles to the plasma membrane in such a way that the concentration of the transporter on the cell surface increases 10- to 40-fold, depending on the cell type and method of measurement (Zorzono et al. (1989) J. Biol. Chem. 264:12358-12363; Holman et al. (1990) J. Biol. Chem. 265:18172-18179; Slot et al. (1991) J. Biol. Chem. 113:123-135; Slot et al. (1991) Proc. Nat'l. Acad. Sci. USA 88:7815-7819; and Smith et al. (1991) Proc. Nat'l. Acad. Sci. USA 88:6893-6897). Glucose uptake is increased proportionally to the increment of GLUT4 molecules in the plasma membrane, suggesting that redistribution of transporters is the main, if not only, mechanism that accounts for this effect, Kandror and Pilch (1994) Proc. Nat'l Acad. Sci USA 91:8017-8021.

Insulin signaling through its receptor tyrosine kinase mediates a phosphatidylinositol 3-kinase-dependent pathway that produces the phosphatidylinositol trisphosphate required for recycling GLUT4 to the plasma membrane (Virkamaki et al., (1999) J. Biol. Chem. 255:4758-4762; White (2002) Am. J. Physiol. 283:E413-E422; Czech et al. (1999) J. Biol. Chem. 274:1865-1868). Among the downstream effectors strongly implicated in linking this signaling pathway to components that regulate GLUT4 trafficking is the protein kinase Akt (also known as protein kinase B) based on studies with cultured cells and a gene knockout mouse model (Jiang et al. (2003) Proc. Natl. Acad. Sci. 100:7569-7574).

Akt is present primarily in two isoforms, Akt1 and Akt2. Akt2 is the predominant isoform expressed in muscle and fat. Recent studies demonstrate an important role of both isoforms in glucose homeostasis. Loss of Akt1 alone slightly impairs insulin-mediated hexose transport activity but has no detectable effect on glycogen synthase kinase (GSK)-3 phosphorylation. In contrast, depletion of Akt2 alone by 70% inhibits approximately half of the insulin responsiveness. Combined depletions of Akt1 plus Akt2 in these cells even more markedly attenuated insulin action on glucose transporter 4 movements, hexose transport activity, and GSK-3 phosphorylation. Studies demonstrate a primary role of Akt2 in insulin signaling, significant functional redundancy of Akt1 and Akt2 isoforms in this pathway, and an absolute requirement of Akt protein kinases for regulation of glucose transport and GSK-3 in cultured adipocytes (Jiang et al. (2003) Proc. Natl. Acad. Sci. 100:7569-7574).

Given the important role of Akt in regulating insulin-responsive translocation of GLUT4, understanding the mechanism by which Akt regulates GLUT4 translocation and the substrates that interact with Akt will allow for the identification of modulators for use in regulating a variety insulin-sensitive cellular responses.

### SUMMARY OF THE INVENTION

The present invention is based, at least in part, on the identification of a heretofore unrecognized biological activity of numerous Akt substrates. In particular, the present invention is based on the discovery that these Akt substrates interact with Akt, an important effector molecule implicated in the regulation of GLUT4 trafficking. These Akt substrates were identified by using a proteomics approach and an anti-Akt substrate antibody.

The present inventors are the first to identify a novel interaction between these Akt substrates and Akt. In particular, the present inventors have demonstrated that these substrates specifically associate with Akt, a critical component of GLUT4 trafficking. Based at least in part on this discovery and on the fact that Akt is important in insulin response and glucose homeostasis, the present invention features methods of identifying insulin response modulators.

Other features and advantages of the invention will be apparent from the following detailed description and claims.

### BRIEF DESCRIPTION OF THE DRAWINGS

FIG. 1 depicts sequence-specific gene silencing by siRNA transfected into 3T3-L1 fibroblasts and 3T3-L1 adipocytes. FIG. 1A depicts sequences of Cy3-labeled lamin A/C and scrambled siRNA duplexes. The lamin A/C sequence is set forth as SEQ ID NO: 1. The scrambled sequence is set forth as SEQ ID NO:2. The FIG. 1B depicts the immunofluorescence of nuclear membrane localization of lamin A/C in 3T3-L1 adipocytes and fibroblasts transfected with Cy3-labeled mouse lamin A/C or scrambled siRNA duplexes. FIG. 1C depicts concentration dependence of lamin A/C gene silencing by siRNA in 3T3-L1 adipocytes.

FIG. 2 depicts time-dependent gene silencing of Akt1 and Akt2 by synthetic siRNA. FIG. 2A depicts synthetic siRNA duplexes targeting different regions of Akt1 and Akt2 mRNAs and used for transfection into 3T3-L1 adipocytes. The akt1a sequence is set forth as SEQ ID NO:3. The akt1b sequence is set forth as SEQ ID NO:4. The akt2a sequence is set forth as SEQ ID NO:5. The akt2b sequence is set forth as SEQ ID NO:6. FIG. 2B depicts western blots of Akt1, Akt2 and Myosin IIB.

FIG. 3 depicts Akt2's primary role in insulin-stimulated Akt protein kinase activity in 3T3-L1 adipocytes. FIG. 3A depicts 3T3-L1 detection of Akt1, Akt2, phosphoT308/309Akt, Acrp30, Myosin IIB in 3T3-L1 adipocytes transfected with scrambled, akt1b, and akt2b siRNA's. FIG. 3B depicts quantification of Akt1 and Akt2 protein levels. FIG. 3C depicts quantification of Akt Thr-308/309 phosphorylation.

FIG. 4 depicts attenuation of insulin-stimulated GSK-3 phosphorylation in 3T3-L1 adipocytes by selective gene silencing. FIG. 4A depicts western blot with phosphor-GSK-3 and GSK antibodies. FIG. 4B depicts western blot with phosphor-Erk-1/2 and Erk-1/2 antibodies. FIG. 4C depicts quantification of phosphorylation of GSK-3α FIG. 4D depicts quantification of phosphorylation of Erk-1/2.

FIG. 5 depicts redundancy of Akt1 and Akt2 in the insulin signaling pathway to hexose transport and GSK-3 phosphorylation in 3T3-L1 adipocytes. FIG. 5A depicts western blot images for Akt protein levels, phosphor-Thr-308/309 levels, phosphor-Ser-21-GSK-3α levels, phosphor-Tyr in IRS proteins, and PKCλ/ξ protein levels in total lysates. FIG. 5B depicts dose dependence of insulin-stimulated deoxyglucose uptake. FIG. 5C depicts inhibition of 100 nM insulin-induced glucose uptake and GSK-3α phosphorylation by knockdown Akt1 and Akt2 protein levels.

FIG. 6 depicts inhibition of insulin-mediated GLUT4 glucose transporter transduction by combined depletion of Akt1 and Akt2 proteins. FIG. 6A depicts representive images for GFP-positive cells and exofacial Myc staining. FIG. 6B depicts Akt protein levels in adipocytes transfected with Myc-GLUT4-EGFP and siRNAs for 48 h. FIG. 6C depicts percentage of the transfected adipocytes showing a Myc-GLUT4-GFP rim on the cell surface. FIG. 6D depicts quantification of the ratio of cell surface Myc signal over the total GFP signal in adipocytes expressing Myc-GLUT4-GFP.

### DETAILED DESCRIPTION OF THE INVENTION

The present invention is based, at least in part, on the discovery of previously unrecognized activity of R-type calcium channel alpha-1E subunit (R-CaC1E), WNK1, FMS interacting protein (FMIP), nGAP-like protein, nuclear matrix protein p84, HIRA interacting protein 3 (HIRIP3), HSP71, ribosomal protein L6, guanine nucleotide exchange factor Lbc (GEF Lbc), ATP citrate lyase, Mi-2b, peripheral benzodiazepine receptor-associated protein 1, heterogeneous nuclear ribonucleoprotein U (hnRNP U protein), pyruvate carboxylase precursor, Eps domain containing protein (RalBP1), nonmuscle myosin IIA (NMMIIA), and stress 70 protein (p66 mot1/GRP75). In particular, these proteins have now been discovered to be substrates of Akt. The present invention is based on the discovery of an interaction between these Akt substrates and Akt, an important effector implicated in the regulation of GLUT4 trafficking.

Generally, insulin is a key regulator of glucose homeostasis, and its absence is lethal in humans. The ability of insulin to lower blood glucose stems in part from its actions in muscle and fat to enhance sugar uptake through regulation of GLUT4. These transporters are sequestered in perinuclear membranes in unstimulated cells and are rapidly induced to recycle to the plasma membrane in response to insulin. Insulin signaling through its receptor tyrosine kinase mediates a phosphatidylinositol 3-kinase-dependent pathway that produces the phosphatidylinositol trisphosphate required for recycling GLUT4 to the plasma membrane. Among the downstream effectors strongly implicated in linking this signaling pathway to components that regulate GLUT4 trafficking is the protein kinase Akt (also known as protein kinase B) based on studies with cultured cells and a gene knockout mouse model (Jiang et al. (2003) Proc. Natl. Acad. Sci. 100:7569-7574, hereby incorporated by reference).

In accordance with the present invention, the Akt substrates identified herein have been found to associate with Akt and therefore may play an important role in GLUT4 trafficking and, more generally, glucose homeostasis. As such, these Akt substrates provide new targets to modulate insulin effects.

Accordingly, the present invention features methods of identifying insulin response modulators. In certain aspects, the present invention provides methods for identifying an insulin response modulator, including contacting a composition comprising, or a cell that expresses Akt or a bioactive fragment thereof and an Akt substrate or a bioactive fragment thereof with a test compound and determining the ability of the test compound to modulate interaction (e.g., binding) of Akt or the Akt bioactive fragment to the Akt substrate or the Akt substrate bioactive fragment, such that the insulin response modulator is identified. In other aspects, the present invention provides methods for identifying an insulin response modulator, including contacting a composition comprising, or a cell that expresses Akt or a bioactive fragment thereof and an Akt substrate or a bioactive fragment thereof with a test compound and determining the ability of the test compound to modulate an activity of Akt or the Akt bioactive fragment, such that an the insulin response modulator is identified. In certain embodiments, the activity of Akt or the bioactive fragment thereof may be selected from the group consisting of regulation of insulin signaling to glycogen synthase kinase, regulation of intracellular GLUT4 trafficking and regulation of intracellular retention of GLUT4. In other aspects, the invention provides methods for identifying an insulin response modulator, including contacting a composition comprising, or a cell that expresses Akt or a bioactive fragment thereof and an Akt substrate or a bioactive fragment thereof with a test compound and determining the ability of the test compound to modulate an activity of the substrate or the substrate bioactive fragment, such that the insulin response modulator is identified. In further aspects, the invention provides a method for identifying an insulin response modulator, including contacting a composition comprising, or a cell that expresses Akt or a bioactive fragment thereof and an Akt substrate or a bioactive fragment thereof with a test compound and determining the ability of the test compound to modulate the phosphorylation state of the Akt substrate or the substrate bioactive fragment, such that the insulin response modulator is identified. In various embodiments of the preceding aspects the modulator identified may be a positive modulator, a negative modulator.

In various embodiments of the preceding aspects of the invention, the Akt substrate may be selected from the group consisting of R-type calcium channel alpha-1E subunit (R-CaC1E), WNK1, FMS interacting protein (FMIP), nGAP-like protein, nuclear matrix protein p84, HIRA interacting protein 3 (HIRIP3), HSP71, ribosomal protein L6, guanine nucleotide exchange factor Lbc (GEF Lbc), ATP citrate lyase, Mi-2b, peripheral benzodiazepine receptor-associated protein 1, heterogeneous nuclear ribonucleoprotein U (hnRNP U protein), pyruvate carboxylase precursor, Eps domain containing protein (RalBP1), nonmuscle myosin IIA (NMMIIA), and stress 70 protein (p66 mot1/GRP75). In other embodiments, the Akt is either Akt1 or Akt2. The Akt bioactive fragment may be any fragment of the Akt substrate having sufficient size and structure to carry out at least one activity (e.g., biological activity) of the corresponding full-length Akt substrate. Exemplary bioactive fragments include, but are not limited to, enzymatic domains, protein binding and/or interaction domains, metal binding domains. Preferred bioactive fragments include regions or domains as described in detail in subsections IA-IQ, infra. The Akt, Akt bioactive fragment, Akt substrate or the substrate bioactive fragment may be detectably labeled, radioactively labeled, or fluorescently labeled. Furthermore, in other embodiments, the interaction, activity, or phosphorylation state may be compared to an appropriate control. In addition, at least one of the Akt, Akt bioactive fragment, Akt substrate or substrate bioactive fragment may be immobilized.

In various embodiments, the activity of the Akt substrate or substrate bioactive fragment is an activity set forth in Table 1 or in subsections IA-IQ, infra. In specific embodiments, the Akt substrate is selected from the group consisting of R-type calcium channel alpha 1E subunit, WNK1, guanine nucleotide exchange factor Lbc (GEF Lbc) or ATP citrate lyase. In yet another embodiment, the Akt substrate is ribosomal protein L6. Bioactive fragments and/or fragment activities (and accordingly, AKT substrate activities) are further described in detail in the references cited throughout subsections IA-IQ, infra. The entire content of these references is incorporated herein by reference.

In the aspects of the present invention where the method involves a cell, the cell may overexpress the Akt substrate or the bioactive fragment thereof, Akt or the bioactive fragment thereof, or both the Akt substrate (or substrate bioactive fragment) and Akt (or Akt bioactive fragment).

In another aspect, the invention provides a modulator as identified by any of the preceding claims. The invention also provides for a pharmaceutical composition including the modulator.

In one aspect, the invention provides a method for identifying an Akt:Akt substrate modulator, including contacting a cell expressing, or a composition comprising, Akt or a bioactive fragment thereof and an Akt substrate or a bioactive fragment thereof with a test compound and determining the ability of the test compound to affect interaction (e.g., binding) of the Akt or the bioactive fragment thereof to the Akt substrate or the bioactive fragment thereof, such that the modulator is identified. In another aspect, the invention provides a method for identifying an Akt:Akt substrate modulator, including contacting a cell expressing, or a composition comprising, Akt or a bioactive fragment thereof and an Akt substrate or a bioactive fragment thereof with a test compound and determining the ability of the test compound to affect activity of the Akt or the bioactive fragment thereof, such that the modulator is identified. In another aspect, the invention provides a method for identifying an Akt:Akt substrate modulator, including contacting a cell expressing, or a composition comprising, Akt or a bioactive fragment thereof and an Akt substrate or a bioactive fragment thereof with a test compound and determining the ability of the test compound to affect activity of the substrate or the bioactive fragment thereof, such that the modulator is identified. In yet another aspect, the invention provides a method for identifying an Akt:Akt substrate modulator, including contacting a cell expressing, or a composition comprising, Akt or a bioactive fragment thereof and an Akt substrate or a bioactive fragment thereof with a test compound and determining the ability of the test compound to affect the phosphorylation state of the Akt substrate or the bioactive fragment thereof, such that the modulator is identified.

In certain embodiments of the preceding aspects, the ability of the test compound to affect, for example, interaction activity or phosphorylation; includes the ability of the test compound to either enhance or inhibit such, for example, interaction activity or phosphorylation. The Akt substrate may be selected from the group consisting of R-type calcium channel alpha-1E subunit (R-CaC1E), WNK1, FMS interacting protein (FMIP), nGAP-like protein, nuclear matrix protein p84, HIRA interacting protein 3 (HIRIP3), HSP71, ribosomal protein L6, guanine nucleotide exchange factor Lbc (GEF Lbc), ATP citrate lyase, Mi-2b, peripheral benzodiazepine receptor-associated protein 1, heterogeneous nuclear ribonucleoprotein U (hnRNP U protein), pyruvate carboxylase precursor, Eps domain containing protein (RalBP1), nonmuscle myosin IIA (NMMIIA), and stress 70 protein (p66 mot1/GRP75). The Akt may be Akt1 or Akt2.

In certain embodiments, the present invention provides methods of modulating insulin responsiveness, regulating glucose transport, regulating gluconeogenesis, regulating glucose homeostasis or regulating blood glucose levels in a subject including administering to the subject an insulin response modulator identified according to the methods of any of the above methods.

In another aspect, the invention provides an antibody that specifically binds to an Akt-interacting domain of an Akt substrate, where the antibody is capable of interfering with the Akt:Akt substrate interaction. The Akt substrate may be selected from the group consisting of R-type calcium channel alpha-1E subunit (R-CaC1E), WNK1, FMS interacting protein (FMIP), nGAP-like protein, nuclear matrix protein p84, HIRA interacting protein 3 (HIRIP3), HSP71, ribosomal protein L6, guanine nucleotide exchange factor Lbc (GEF Lbc), ATP citrate lyase, Mi-2b, peripheral benzodiazepine receptor-associated protein 1, heterogeneous nuclear ribonucleoprotein U (hnRNP U protein), pyruvate carboxylase precursor, Eps domain containing protein (RalBP1), nonmuscle myosin IIA (NMMIIA), or stress 70 protein (p66 mot1/GRP75). The invention further provides for a pharmaceutical composition including the antibody.

In yet another aspect, the present invention provides a pharmaceutical composition including an Akt-interacting domain of an Akt substrate, where the Akt-interacting domain is capable of interfering with the Akt:Akt substrate interaction. The substrate may be R-type calcium channel alpha-1E subunit (R-CaC1E), WNK1, FMS interacting protein (FMIP), nGAP-like protein, nuclear matrix protein p84, HIRA interacting protein 3 (P3), HSP71, ribosomal protein L6, guanine nucleotide exchange factor Lbc (GEF Lbc), ATP citrate lyase, Mi-2b, peripheral benzodiazepine receptor-associated protein 1, heterogeneous nuclear ribonucleoprotein U (hnRNP U protein), pyruvate carboxylase precursor, Eps domain containing protein (RalBP1), nonmuscle myosin IIA (NMMIIA), or stress 70 protein (p66 mot1/GRP75).

In other aspects, the present invention provides methods for treating an insulin response disease or disorder (e.g., Type I diabetes, Type II diabetes, insulin resistance) including administering any of the pharmaceutical compositions described above.

Various aspects of the invention are described in further detail in the following subsections:

I. Substrates:

According to the invention, several substrates have been identified as interacting with Akt in regulating the insulin response of a cell. These Akt substrates include R-CaC1E, WNK1, FMIP, nGAP-like protein, THO complex 1, HIRIP3, HSP71, ribosomal protein L6, GEF Lbc, ATP citrate lyase, Mi-2b, KIAA0612, AAH07950, pyruvate carboxylase, Eps domain containing protein, nonmuscle myosin IIA (NMMIIA), and p66 mot1/GRP75. Table 1 summarizes the Akt substrates and their putative Akt phosphorylation sites. Using methods described in the present disclosure, use of any one of these substrates in appropriate screening assays would provide for the identification of insulin response modulators.

TABLE 1
Summary of Akt substrates

| | | | Putative Akt Phosphorylation Sites | | | | | |
|---|---|---|---|---|---|---|---|---|
| GI Acc # | Name | Description | p-site 1 | p-site2 | p-site3 | p-site4 | p-site5 | psite6 |
| 399202 | R-CaC1E | R-type calcium | T2212 | S932 | T1059 | S934 | S2033 | S2084 |
| | | channel 1E | | | | | | |
| 16758634 | WNK1 | A Serine/Threonine | T58 | S1884 | T1989 | T1872 | S563 | |
| | | protein kinase | (Akt & | | | | (pkcz) | |
| | | | pka) | | | | | |
| 24980875 | FMIP | Fms interacting | T30 | T328 | | | | |
| | | protein, interacting | | | | | | |
| | | with tyrosin kinase | | | | | | |
| | | receptors | | | | | | |
| 14009346 | nGAP-like | similar to Ras | S907 | S984 | T212 | | | |
| | protein | GTPase activating | | | | | | |
| | | protein | | | | | | |
| 23956332 | THO | protein containing a | S9 | S240 | T259 | T153 | S504 | |
| | complex 1 | death domain with | | | | | | |
| | | unknown function | | | | | | |
| 21396500 | HIRIP3 | HIRA interacting | S98 | T509 | S400 | S398 | | |
| | | protein, may | | | | | | |
| | | regulate chromatin | | | | | | |
| | | and histone | | | | | | |
| | | metabolism | | | | | | |
| 20820471 | HSP71 | a heat shock protein | S275 | T265 low | | | | |
| | | | low | | | | | |
| 16758864 | ribosomal | L6 can be | S72 | S141 | | | | |
| | protein L6 | phosphorylated by | | | | | | |
| | | s6 kinase and | | | | | | |
| | | recognized by anti- | | | | | | |
| | | PAS antibody | | | | | | |
| 15207794 | GEF Lbc | Guanine nucleotide | S1602 | S2237 | | | | |
| | | exchange factor | | (pkcz) | | | | |
| 8392839 | ATP citrate | Succinyl-CoA | S454 | S978 | S665 | | | |
| | lyase | synthetase, reported | | | (pkcz) | | | |
| | | to be AKT substrate | | | | | | |
| 4557453 | Mi-2b | chromodomain | S310 | multiple | | | | |
| | | helicase DNA | | PKCz | | | | |
| | | binding protein 4, | | sites | | | | |
| | | exist in complex | | | | | | |
| | | containing | | | | | | |
| | | deacetylase | | | | | | |
| 7513043 | KIAA0612 | hypothetical protein, | S90 | S1160 | T847 | | | |
| | | similar to peripheral | | | | | | |
| | | benzodiazapine | | | | | | |
| | | receptor associated | | | | | | |
| | | protein 1 and RIM- | | | | | | |
| | | binding protein 1 | | | | | | |
| 14044052 | AAH07950 | Similar to | S193 | S154 low | | | | |
| | | heterogeneous | Low | | | | | |
| | | nuclear | | | | | | |
| | | ribonucleoprotein U | | | | | | |
| | | (scaffold attachment | | | | | | |
| | | factor A) | | | | | | |
| 200246 | pyruvate | | S50 | | | | | |
| | carboxylase | | | | | | | |
| 6677715 | Eps domain | associate with | S509 | S656 | S484 | | | |
| | containing | RalBP1 | | | | | | |
| | protein | | | | | | | |
| 20137006 | NMMIIA | nonmuscle myosin | | | | | | |
| | | IIA | | | | | | |
| 6754256 | p66 | stress 70 protein; | S662 | S664 low | T463 | | | |
| | mot1/GRP75 | dnaK-type | low | | low | | | |
| | | chaperone | | | | | | |

IA. R-type Calcium Channel Alpha-1E Subunit (R-CaC1E)

R-type Calcium Channel Alpha-1E Subunit (R-CaC1E) has been identified as an Akt substrate that interacts with Akt. Human calcium channel, voltage-dependent, alpha 1E subunit is associated with reference sequence NP_{—}000712 (GenBank Accession No. 4502529). Other relevant sequences include rabbit R-CaC1E (GenBank Accession No. 399202).

NP_000712. calcium channel, voltage-dependent,
alpha 1E subunit [gi: 4502529]

| | |
|---|---|
| LOCUS | NP_000712 2251 aa linear PRI 06-OCT-2003 |
| DEFINITION | calcium channel, voltage-dependent, alpha 1E subunit [Homo |
| | sapiens]. |
| ACCESSION | NP_000712 |
| VERSION | NP_000712.1 GI: 4502529 |
| DBSOURCE | REFSEQ: accession NM_000721.1 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 2251) |
| AUTHORS | Diriong, S., Lory, P., Williams, M. E., Ellis, S. B., Harpold, M. M. and |
| | Taviaux, S. |
| TITLE | Chromosomal localization of the human genes for alpha 1A, alpha 1B, |
| | and alpha 1E voltage-dependent Ca2+ channel subunits |
| JOURNAL | Genomics 30 (3), 605-609 (1995) |
| MEDLINE | 96423049 |
| PUBMED | 8825650 |
| REFERENCE | 2 (residues 1 to 2251) |
| AUTHORS | Williams, M. E., Marubio, L. M., Deal, C. R., Hans, M., Brust, P. F., |
| | Philipson, L. H., Miller, R. J., Johnson, E. C., Harpold, M. M. and |
| | Ellis, S. B. |
| TITLE | Structure and functional characterization of neuronal alpha 1E |
| | calcium channel subtypes |
| JOURNAL | J. Biol. Chem. 269 (35), 22347-22357 (1994) |
| MEDLINE | 94350992 |
| PUBMED | 8071363 |
| REFERENCE | 3 (residues 1 to 2251) |
| AUTHORS | Schneider, T., Wei, X., Olcese, R., Costantin, J. L., Neely, A., |
| | Palade, P., Perez-Reyes, E., Qin, N., Zhou, J., Crawford, G. D. et al. |
| TITLE | Molecular analysis and functional expression of the human type E |
| | neuronal Ca2+ channel alpha 1 subunit |
| JOURNAL | Recept. Channels 2 (4), 255-270 (1994) |
| MEDLINE | 95236033 |
| PUBMED | 7536609 |
| REFERENCE | 4 (residues 1 to 2251) |
| AUTHORS | Soong, T. W., Stea, A., Hodson, C. D., Dubel, S. J., Vincent, S. R. and |
| | Snutch, T. P. |
| TITLE | Structure and functional expression of a member of the low |
| | voltage-activated calcium channel family |
| JOURNAL | Science 260 (5111), 1133-1136 (1993) |
| MEDLINE | 93262464 |
| PUBMED | 8388125 |
| COMMENT | PROVISIONAL REFSEQ: This record has not yet been subject to final |
| | NCBI review. The reference sequence was derived from L29384.1. |
| FEATURES | Location/Qualifiers |
| source | 1 . . . 2251 |
| | /organism=“Homo sapiens” |
| | /db_xref=“taxon:9606” |
| | /chromosome=“1” |
| | /map=“1q25-q31” |
| Protein | 1 . . . 2251 |
| | /product=“calcium channel, voltage-dependent, alpha 1E |
| | subunit” |
| Region | 124 . . . 350 |
| | /region_name=“Ion transport protein. This family contains |
| | Sodium, Potassium, Calcium ion channels. This family is 6 |
| | transmembrane helices in which the last two helices flank |
| | a loop which determines ion selectivity. In some |
| | sub-families (e.g. Na channels) the domain is repeated |
| | four times, whereas in others (e.g. K channels) the |
| | protein forms as a tetramer in the membrane. A bacterial |
| | structure of the protein is known for the last two helices |
| | but is not the Pfam family due to it lacking the first |
| | four helices” |
| | /note=“ion_trans” |
| | /db_xref=“CDD:pfam00520” |
| Region | 523 . . . 701 |
| | /region_name=“Ion transport protein. This family contains |
| | Sodium, Potassium, Calcium ion channels. This family is 6 |
| | transmembrane helices in which the last two helices flank |
| | a loop which determines ion selectivity. In some |
| | sub-families (e.g. Na channels) the domain is repeated |
| | four times, whereas in others (e.g. K channels) the |
| | protein forms as a tetramer in the membrane. A bacterial |
| | structure of the protein is known for the last two helices |
| | but is not the Pfam family due to it lacking the first |
| | four helices” |
| | /note=“ion_trans” |
| | /db_xref=“CDD:pfam00520” |
| Region | 1166 . . . 1366 |
| | /region_name=“Ion transport protein. This family contains |
| | Sodium, Potassium, Calcium ion channels. This family is 6 |
| | transmembrane helices in which the last two helices flank |
| | a loop which determines ion selectivity. In some |
| | sub-families (e.g. Na channels) the domain is repeated |
| | four times, whereas in others (e.g. K channels) the |
| | protein forms as a tetramer in the membrane. A bacterial |
| | structure of the protein is known for the last two helices |
| | but is not the Pfam family due to it lacking the first |
| | four helices” |
| | /note=“ion_trans” |
| | /db_xref=“CDD:pfam00520” |
| Region | 1490 . . . 1703 |
| | /region_name=“Ion transport protein. This family contains |
| | Sodium, Potassium, Calcium ion channels. This family is 6 |
| | transmembrane helices in which the last two helices flank |
| | a loop which determines ion selectivity. In some |
| | sub-families (e.g. Na channels) the domain is repeated |
| | four times, whereas in others (e.g. K channels) the |
| | protein forms as a tetramer in the membrane. A bacterial |
| | structure of the protein is known for the last two helices |
| | but is not the Pfam family due to it lacking the first |
| | four helices” |
| | /note=“ion_trans” |
| | /db_xref=“CDD:pfam00520” |
| variation | 2095 |
| | /allele=“S” |
| | /allele=“G” |
| | /db_xref=“dbSNP:2480373” |
| CDS | 1 . . . 2251 |
| | /gene=“CACNA1E” |
| | /coded_by=“NM_000721.1:166..6921” |
| | /note=“brain specific; |
| | go_component: voltage-gated calcium channel complex [goid |
| | 0005891] [evidence TAS] [pmid 8071363]; |
| | go_component: integral to membrane [goid 0016021] |
| | [evidence IEA]; |
| | go_function: voltage-gated calcium channel activity [goid |
| | 0005245] [evidence TAS] [pmid 8071363]; |
| | go_function: calcium ion binding [goid 0005509] [evidence |
| | IEA]; |
| | go_process: small molecule transport [goid 0006832] |
| | [evidence TAS] [pmid 8071363]; |
| | go_process: synaptic transmission [goid 0007268] [evidence |
| | TAS] [pmid 8071363]; |
| | go_process: cation transport [goid 0006812] [evidence |
| | IEA]; |
| | go_process: calcium ion transport [goid 0006816][evidence |
| | IEA]” |
| | /db_xref=“GeneID:777” |
| | /db_xref=“LocusID:777” |
| | /db_xref=“MIM:601013” |

Origin

| [SEQ ID NO: 7] | |
|---|---|
| 1 | marfgeavva rpgsgdgdsd qsrnrqgtpv pasgqaaayk qtkaqrartm alynpipvrq |
| 61 | ncftvnrslf ifgednivrk yakklidwpp feymilatii ancivlaleq hlpeddktpm |
| 121 | srrlektepy figifcfeag ikivalgfif hkgsylrngw nvmdfivvls gilatagthf |
| 181 | nthvdlrtlr avrvlrplkl vsgipslqiv lksimkamvp liqigiliff ailmfaiigl |
| 241 | efysgklhra cfmnnsgile gfdpphpcgv qgcpagyeck dwigpndgit qfdnilfavl |
| 301 | tvfqcitmeg wttvlyntnd algatwnwly fipliiigsf fvlnlvlgvl sgefakerer |
| 361 | venrrafmkl rrqqqierel ngyrawidka eevmlaeenk nagtsalevl rratikrsrt |
| 421 | eamtrdssde hcvdissvgt plarasiksa kvdgvsyfrh kerlirisir hmvksqvfyw |
| 481 | ivlslvalnt acvaivhhnq pqwlthllyy aeflflglfl lemslkmygm gprlyfhssf |
| 541 | ncfdfgvtvg sifevvwaif rpgtsfgisv irairlirif kitkywaslr nlvvslmssm |
| 601 | ksiisllfll flfivvfall gmqlfggrfn fndgtpsanf dtfpaaimtv fqiltgedwn |
| 661 | evmyngirsq ggvssgmwsa iyfivltlfg nytllnvfla iavdnlanaq eltkdeqeee |
| 721 | eafnqkhalq kakevspmsa pnmpsierer rrrhhmsvwe qrtsqlrkhm qmssqealnr |
| 781 | eeaptmnpln plnplsslnp lnahpslyrr praieglalg lalekfeeer isrggslkgd |
| 841 | ggdrssaldn qrtplslgqr eppwlarpch gncdptqqea gggeavvtfe drarhrqsqr |
| 901 | rsrhrrvrte gkesssasrs rsasqersld eamptegekd helrgnhgak eptiqeeraq |
| 961 | dlrrtnslmv srgsglaggl deadtplvlp hpelevgkhv viteqepegs seqallgnvq |
| 1021 | ldmgrvisqs epdlscitan tdkattests vtvaipdvdp lvdstvvhis nktdgeaspl |
| 1081 | keaeiredee evekkkqkke kretgkamvp hssmfifstt npirrachyi vnlryfemci |
| 1141 | llviaassia laaedpvltn sernkvlryf dyvftgvftf emvikmidqg lilqdgsyfr |
| 1201 | dlwnildfvv vvgalvafal analgtnkgr diktikslrv lrvlrplkti krlpklkavf |
| 1261 | dcvvtslknv fnilivyklf mfifaviavq lfkgkffyct dsskdtekec ignyvdhekn |
| 1321 | kmevkgrewk rhefhydnii walltlftvs tgegwpqvlq hsvdvteedr gpsrsnrmem |
| 1381 | sifyvvyfvv fpfffvnifv aliiitfqeq gdkmmeecsl ekneracidf aisakpltry |
| 1441 | mpqnrhtfqy rvwhfvvsps feytimamia lntvvlmmky ysapctyela lkylniaftm |
| 1501 | vfslecvlkv iafgflnyfr dtwnifdfit vigsiteiil tdsklvntsg fnmsflklfr |
| 1561 | aarlikllrq gytirillwt fvqsfkalpy vclliamlff iyaiigmqvf gnikldeesh |
| 1621 | inrhnnfrsf fgslmllfrs atgeawqeim lsclgekgce pdttapsgqn enercgtdla |
| 1681 | yvyfvsfiff csflmlnlfv avimdnfeyl trdssilgph hldefvrvwa eydraacgri |
| 1741 | hytemyemlt lmspplglgk rcpskvaykr lvlmnmpvae dmtvhftstl malirtaldi |
| 1801 | kiakggadrq qldselqket laiwphlsqk mldllvpmpk asdltvgkiy aammimdyyk |
| 1861 | qskvkkqrqq leeqknapmf qrmepsslpq elianakaip ylqqdpvsgl sgrsgypsms |
| 1921 | plspqdifql acmdpaddgq fqerqslvvt dpssmrrsfs tirdkrsnss wleefsmers |
| 1981 | sentyksrrr syhsslrlsa hrlnsdsghk sdthpsggre rrrskerkhl lspdvsrcns |
| 2041 | eergtqadwe sperrqsrsp segrsqtpnr qgtgslsess ipsvsdtstp rrsrrqlppv |
| 2101 | ppkprpllsy sslirhagsi sppadgseeg spltsqales nnawltessn sphpqqrqha |
| 2161 | spqryisepy lalhedshas dcveeetltf eaavatslgr sntigsappl rhswqmpngh |
| 2221 | yrrrrrggpg pgmmcgavnn llsdteeddk c |

IB. WNK1

WNK1 has been identified as an Akt substrate that interacts with Akt. Human WNK 1 is associated with reference sequence NP_{—}061852 (GenBank Accession No. 12711660). WNK1 is identified and characterized in Verissimo et al. Oncogene (2001) 20(39):5562-5569. Other relevant sequences include rat WNK1 associated with reference sequence NP_{—}446246 (GenBank Accession No. 16758634).

NP_061852. protein kinase, 1...[gi: 12711660]

| | |
|---|---|
| LOCUS | NP_061852 2382 aa linear PRI 05-OCT-2003 |
| DEFINITION | protein kinase, lysine deficient 1; kinase deficient protein [Homo |
| | sapiens]. |
| ACCESSION | NP_061852 |
| VERSION | NP_061852.1 GI: 12711660 |
| DBSOURCE | REFSEQ: accession NM_018979.1 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 2382) |
| AUTHORS | Xu, B. E., Min, X., Stippec, S., Lee, B. H., Goldsmith, E. J. and Cobb, M. H. |
| TITLE | Regulation of WNK1 by an autoinhibitory domain and |
| | autophosphorylation |
| JOURNAL | J. Biol. Chem. 277 (50), 48456-48462 (2002) |
| MEDLINE | 22359054 |
| PUBMED | 12374799 |
| REMARK | GeneRIF: identification of autoinhibitory domain |
| REFERENCE | 2 |
| AUTHORS | Verissimo, F. and Jordan, P. |
| TITLE | WNK kinases, a novel protein kinase subfamily in multi-cellular |
| | organisms |
| JOURNAL | Oncogene 20 (39), 5562-5569 (2001) |
| MEDLINE | 21455683 |
| PUBMED | 11571656 |
| REFERENCE | 3 (residues 1 to 2382) |
| AUTHORS | Wilson, F. H., Disse-Nicodeme, S., Choate, K. A., Ishikawa, K., |
| | Nelson-Williams, C., Desitter, I., Gunel, M., Milford, D. V., |
| | Lipkin, G. W., Achard, J. M., Feely, M. P., Dussol, B., Berland, Y., |
| | Unwin, R. J., Mayan, H., Simon, D. B., Farfel, Z., Jeunemaitre, X. and |
| | Lifton, R. P. |
| TITLE | Human hypertension caused by mutations in WNK kinases |
| JOURNAL | Science 293 (5532), 1107-1112 (2001) |
| MEDLINE | 21390047 |
| PUBMED | 11498583 |
| REFERENCE | 4 (residues 1 to 2382) |
| AUTHORS | Moore, T. M., Garg, R., Johnson, C., Coptcoat, M. J., Ridley, A. J. and |
| | Morris, J. D. |
| TITLE | PSK, a novel STE20-like kinase derived from prostatic carcinoma |
| | that activates the c-Jun N-terminal kinase mitogen-activated |
| | protein kinase pathway and regulates actin cytoskeletal |
| | organization |
| JOURNAL | J. Biol. Chem. 275 (6), 4311-4322 (2000) |
| MEDLINE | 20127920 |
| PUBMED | 10660600 |
| COMMENT | PROVISIONAL REFSEQ: This record has not yet been subject to final |
| | NCBI review. The reference sequence was derived from AJ296290.1. |
| | Summary: The WNK1 gene encodes a cytoplasmic serine-threonine |
| | kinase expressed in distal nephron.[supplied by OMIM]. |
| FEATURES | Location/Qualifiers |
| source | 1 . . . 2382 |
| | /organism=“Homo sapiens” |
| | /db_xref=“taxon:9606” |
| | /chromosome=“12” |
| | /map=“12p13.3” |
| Protein | 1 . . . 2382 |
| | /product=“protein kinase, lysine deficient 1” |
| | /note=“kinase deficient protein” |
| variation | 118 |
| | /allele=“D” |
| | /allele=“E” |
| | /db_xref=“dbSNP:11885” |
| Region | 226 . . . 479 |
| | /region_name=“Serine/Threonine protein kinases, catalytic |
| | domain” |
| | /note=“S_TKc” |
| | /db_xref=“CDD:smart00220” |
| variation | 531 |
| | /allele=“N” |
| | /allele=“D” |
| | /db_xref=“dbSNP:3178414” |
| variation | 665 |
| | /allele=“I” |
| | /allele=“T” |
| | /db_xref=“dbSNP:2286007” |
| variation | 1056 |
| | /allele=“P” |
| | /allele=“T” |
| | /db_xref=“dbSNP:956868” |
| variation | 1506 |
| | /allele=“S” |
| | /allele=“C” |
| | /db_xref=“dbSNP:7955371” |
| CDS | 1 . . . 2382 |
| | /gene=“PRKWNK1” |
| | /coded_by=“NM_018979.1:1..7149” |
| | /db_xref=“GeneID:65125” |
| | /db_xref=“LocusID:65125” |
| | /db_xref=“MIM:605232” |

Origin

| [SEQ ID NO: 8] | |
|---|---|
| 1 | msggaaekqs stpgslflsp papapkngss sdssvgeklg aaaadavtgr teeyrrrrht |
| 61 | mdkdsrgaaa tttttehrff rrsvicdsna talelpglpl slpqpsipaa vpqsappeph |
| 121 | reetvtatat sqvaqqppaa aapgeqavag papstvpsst skdrpvsqps lvgskeeppp |
| 181 | arsgsgggsa kepqeersqq qddieeletk avgmsndgrf lkfdieigrg sfktvykgld |
| 241 | tettvevawc elqdrkltks erqrfkeeae mlkglqhpni vrfydswest vkgkkcivlv |
| 301 | telmtsgtlk tylkrfkvmk ikvlrswcrq ilkglqflht rtppiihrdl kcdnifitgp |
| 361 | tgsvkigdlg latlkrasfa ksvigtpefm apemyeekyd esvdvyafgm cmlematsey |
| 421 | pysecqnaaq iyrrvtsgvk pasfdkvaip evkeiiegci rqnkderysi kdllnhaffq |
| 481 | eetgvrvela eeddgekiai kiwiriedik klkgkykdne aiefsfdler dvpedvaqem |
| 541 | vesgyvcegd hktmakaikd rvslikrkre qrqlvreeqe kkkqeesslk qqveqssasq |
| 601 | tgikqlpsas tgiptastts asvstqvepe epeadqhqql qyqqpsisvl sdgtvdsgqg |
| 661 | ssvftesrvs sqqtvsygsq heqahstgtv pghipstvqa qsqphgvypp ssvaqgqsqg |
| 721 | qpssssltgv sssqpiqhpq qqqgiqqtap pqqtvqysls qtstsseatt aqpvsqpqap |
| 781 | qvlpqvsagk qlpvsqpvpt iqgepqipva tqpsvvpvhs gahflpvgqp lptpllpqyp |
| 841 | vsqipistph vstaqtgfss lpitmaagit qplltlassa ttaaipgvst vvpsqlptll |
| 901 | qpvtqlpsqv hpqllqpavq smgipanlgq aaevplssgd vlyqgfpprl ppqypgdsni |
| 961 | apssnvasvc ihstvlsppm ptevlatpgy fptvvqpyve snllvpmggv ggqvqvsqpg |
| 1021 | gslaqaptts sqqavlestq gvsqvapaep vavaqpqatq pttlassvds ahsdvasgms |
| 1081 | dgnenvpsss grhegrttkr hyrksvrsrs rhektsrpkl rilnvsnkgd rvvecqleth |
| 1141 | nrkmvtfkfd ldgdnpeeia timvnndfil aieresfvdq vreiiekade mlsedvsvep |
| 1201 | egdqgleslq gkddygfsgs qklegefkqp ipassmpqqi giptssltqv vhsagrrfiv |
| 1261 | spvpesrlre skvfpseitd tvaastaqsp gmnlshsass lslqqafsel rraqmtegpn |
| 1321 | tappnfshtg ptfpvvppfl ssiagvptta aatapvpats sppndistsv iqsevtvpte |
| 1381 | egiagvatst gvvtsgglpi ppvsespvls svvssitipa vvsisttsps lqvptstsei |
| 1441 | vvsstalyps vtvsatsasa ggstatpgpk ppavvsqqaa gsttvgatlt svstttsfps |
| 1501 | tasqlsiqls sststptlae tvvvsahsld ktshssttgl afslsapsss sspgagvssy |
| 1561 | isqpgglhpl vipsviastp ilpqaagpts tpllpqvpsi pplvajvanv pavqqtlihs |
| 1621 | qpqpallpnq phthcpevds dtqpkapgid diktleeklr slfsehsssg aqhasvslet |
| 1681 | slviestvtp gipttavaps klltsttstc lpptnlplgt valpvtpvvt pgqvstpvst |
| 1741 | ttsgvkpgta pskppltkap vlpvgtelpa gtlpseqlpp fpgpsltqsq qpledldaql |
| 1801 | rrtlspeiit vtsavgpvsm aaptaiteag tqpqkgvsqv kegpvlatss gagvfkmgrf |
| 1861 | qvsvaadgaq kegknkseda ksvhfessts essvlssssp estlvkpepn gitipgissd |
| 1921 | vpesahktta seaksdtgqp tkvgrfqvtt tankvgrfsv sktedkitdt kkegpvaspp |
| 1981 | fmdleqavlp avipkkekpe lsepshlngp ssdpeaafis rdvddgsgsp hsphqlssks |
| 2041 | lpsqnlsqsl snsfnssyms sdnesdiede dlklelrrlr dkhlkeiqdl qsrqkheies |
| 2101 | lytklgkvpp aviippaapl sgrrrrptks kgskssrsss lgnkspqlsg nlsgqsaasv |
| 2161 | lhpqqtlhpp gnipesgqnq llqplkpsps sdnlysafts dgaisvpsls apgqgtsstn |
| 2221 | tvgatvnsqa aqaqppamts srkgtftddl hklvdnward amnlsgrrgs kghmnyegpg |
| 2281 | markfsapgq lcismtsnlg gsapisaasa tslghftksm cppqqygfpa tpfgaqwsgt |
| 2341 | ggpapqplgq fqpvgtaslq nfnisnlqks isnppgsnlr tt |

IC. Fms Interacting Protein (FMIP)

Fms interacting protein (FMIP) has been identified as an Akt substrate that interacts with Akt. Human FMIP is associated with reference sequence NP_{—}003669 (GenBank Accession No. 19923178). Other relevant sequences include mouse FMIP (GenBank Accession No. 24980875).

NP_003669. Chromosome 22 open reading frame 19 [gi: 19923178]

| | |
|---|---|
| LOCUS | NP_003669 683 aa linear PRI 05-OCT-2003 |
| DEFINITION | chromosome 22 open reading frame 19; gene from NF2/meningioma |
| | region of 22q12 [Homo sapiens]. |
| ACCESSION | NP_003669 |
| VERSION | NP_003669.2 GI: 19923178 |
| DBSOURCE | REFSEQ: accession NM_003678.2 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 683) |
| AUTHORS | Tamura, T., Mancini, A., Joos, H., Koch, A., Hakim, C., Dumanski, J., |
| | Weidner, K. M. and Niemann, H. |
| TITLE | FMIP, a novel Fms-interacting protein, affects |
| | granulocyte/macrophage differentiation |
| JOURNAL | Oncogene 18 (47), 6488-6495 (1999) |
| MEDLINE | 20065119 |
| PUBMED | 10597251 |
| REFERENCE | 2 (residues 1 to 683) |
| AUTHORS | Xie, Y. G., Han, F. Y., Peyrard, M., Ruttledge, M. H., Fransson, I., |
| | DeJong, P., Collins, J., Dunham, I., Nordenskjold, M. and Dumanski, J. P. |
| TITLE | Cloning of a novel, anonymous gene from a megabase-range YAC and |
| | cosmid contig in the neurofibromatosis type 2/meningioma region on |
| | human chromosome 22q12 |
| JOURNAL | Hum. Mol. Genet. 2 (9), 1361-1368 (1993) |
| MEDLINE | 94061029 |
| PUBMED | 8242058 |
| COMMENT | PROVISIONAL REFSEQ: This record has not yet been subject to final |
| | NCBI review. The reference sequence was derived from AB023200.1. |
| | On Apr 4. 2002 this sequence version replaced gi: 4505829. |
| FEATURES | Location/Qualifiers |
| source | 1 . . . 683 |
| | /organism=“Homo sapiens” |
| | /db_xref=“taxon:9606” |
| | /chromosome=“22” |
| | /map=“22q12” |
| Protein | 1 . . . 683 |
| | /product=“chromosome 22 open reading frame 19” |
| | /note=“gene from NF2/meningioma region of 22q12” |
| variation | 475 |
| | /allele=“S” |
| | /allele=“T” |
| | /db_xref=“dbSNP:8141153” |
| variation | 525 |
| | /allele=“V” |
| | /allele=“I” |
| | /db_xref=“dbSNP:737976” |
| variation | 579 |
| | /allele=“V” |
| | /allele=“I” |
| | /db_xref=“dbSNP:1049534” |
| CDS | 1 . . . 683 |
| | /gene=“C22orf19” |
| | /coded_by=“NM_003678.2:56..2107” |
| | /note=“go_function: tumor suppressor [goid 0008181] |
| | [evidence TAS] [pmid 8242058]” |
| | /db_xref=“GeneID:8563” |
| | /db_xref=“LocusID:8563” |

Origin

| [SEQ ID NO: 9] | |
|---|---|
| 1 | mssesskkrk pkvirsdgap aegkrnrsdt eqegkyysee aevdlrdpgr dyelykytcq |
| 61 | elqrlmaeiq dlksrggkdv aieieerriq scvhfmtlkk inriahirik kgrdqtheak |
| 121 | qkvdayhlql qnllyevmhl qkeitkclef kskheeidlv sleefykeap pdiskaevtm |
| 181 | gdphqqtlar ldweleqrkr laekyrecls nkekilkeie vkkeylsslq prlnsimqas |
| 241 | lpvqeylfmp fdqahkqyet arhlppplyv lfvqataygq acdktlsvai egsvdeakal |
| 301 | fkppedsqdd esdsdaeeeq ttkrrrptlg vqlddkrkem lkrhplsvml dlkckddsvl |
| 361 | hltfyylmnl nimtvkakvt tamelitpis agdllspdsv lsclypgdhg kktpnpanqy |
| 421 | qfdkvgiltl sdyvlelghp ylwvqklggl hfpkeqpqqt viadhslsas hmettmkllk |
| 481 | trvqsrlalh kqfaslehgi vpvtsdcqyl fpakvvsrlv kwvtiahedy melhftkdiv |
| 541 | daglagdtnl yymaliergt aklqaavvln pgyssippvf qlclnwkgek tnsnddnira |
| 601 | megevnvcyk elcgpwpshq lltnqlqrlc vlldvylete shddsvegpk efpqekmclr |
| 661 | lfrgpsrmkp fkynhpqgff shr |

ID. nGAP-like Protein

nGAP-like protein has been identified as an Akt substrate that interacts with Akt. Human nGAP-like protein is associated with reference sequence NP_{—}115941 (GenBank Accession No. 20070109). A specific nGAP-like protein sequence is associated with GenBank Accession No. 14009346.

NP_115941. DAB2 interacting protein [gi: 20070109]

| | |
|---|---|
| LOCUS | NP_115941 967 aa linear PRI 05-OCT-2003 |
| DEFINITION | DAB2 interacting protein; nGAP-like protein; DOC-2/DAB2 interactive |
| | protein [Homo sapiens]. |
| ACCESSION | NP_115941 |
| VERSION | NP_115941.1 GI: 20070109 |
| DBSOURCE | REFSEQ: accession NM_032552.1 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 967) |
| AUTHORS | Chen, H., Toyooka, S., Gazdar, A. F. and Hsieh, J. T. |
| TITLE | Epigenetic regulation of a novel tumor suppressor gene (hDAB2IP) in |
| | prostate cancer cell lines |
| JOURNAL | J. Biol. Chem. 278 (5), 3121-3130 (2003) |
| MEDLINE | 22439816 |
| PUBMED | 12446720 |
| REMARK | GeneRIF: Epigenetic regulation of this novel tumor suppressor gene |
| | in prostate cancer cell lines. |
| REFERENCE | 2 (residues 1 to 967) |
| AUTHORS | Chen, H., Pong, R. C., Wang, Z. and Hsieh, J. T. |
| TITLE | Differential regulation of the human gene DAB2IP in normal and |
| | malignant prostatic epithelia: cloning and characterization |
| JOURNAL | Genomics 79 (4), 573-581 (2002) |
| MEDLINE | 21945266 |
| PUBMED | 11944990 |
| REMARK | GeneRIF: Normal prostatic epithelial cells have elevated DAB2IP |
| | mRNA compared with cancer cells, which correlates with increased |
| | DAB2IP promoter activity. |
| REFERENCE | 3 (residues 1 to 967) |
| AUTHORS | Wang, Z., Tseng, C. P., Pong, R. C., Chen, H., McConnell, J. D., Navone, N. |
| | and Hsieh, J. T. |
| TITLE | The mechanism of growth-inhibitory effect of DOC-2/DAB2 in prostate |
| | cancer. Characterization of a novel GTPase-activating protein |
| | associated with N-terminal domain of DOC-2/DAB2 |
| JOURNAL | J. Biol. Chem. 277 (15), 12622-12631 (2002) |
| MEDLINE | 21935348 |
| PUBMED | 11812785 |
| COMMENT | PROVISIONAL REFSEQ: This record has not yet been subject to final |
| | NCBI review. The reference sequence was derived from AF367051.1. |
| FEATURES | Location/Qualifiers |
| source | 1 . . . 967 |
| | /organism=“Homo sapiens” |
| | /db_xref=“taxon:9606” |
| | /chromosome=“9” |
| | /map=“9q33.1-q33.3” |
| Protein | 1 . . . 967 |
| | /product=“DAB2 interacting protein” |
| | /note=“nGAP-like protein; DOC-2/DAB2 interactive protein” |
| Region | 21 . . . 117 |
| | /region_name=“Protein kinase C conserved region 2 (CalB)” |
| | /note=“C2” |
| | /db_xref=“CDD:smart00239” |
| Region | 143 . . . 456 |
| | /region_name=“GTPase-activator protein for Ras-like |
| | GTPases” |
| | /note=“RasGAP” |
| | /db_xref=“CDD:smart00323” |
| Region | 836 . . . 964 |
| | /region_name=“Chromosome segregation ATPases [Cell |
| | division and chromosome partitioning]” |
| | /note=“Smc” |
| | /db_xref=“CDD:COG1196” |
| CDS | 1 . . . 967 |
| | /gene=“DAB2IP” |
| | /coded_by=“NM_032552.1:306..3209” |
| | /note=“DIP1/2” |
| | /db_xref=“GeneID:153090” |
| | /db_xref=“LocusID:153090” |

Origin

| [SEQ ID NO: 10] | |
|---|---|
| 1 | menlrravhp nkdnsrrveh ilklwvieak dlpakkkylc elclddvlya rttgklktdn |
| 61 | vfwgehfefh nlpplrtvtv hlyretdkkk kkernsylgl vslpaasvag rqfvekwypv |
| 121 | vtpnpkggkg pgpmirikar yqtitilpme mykefaehit nhylglcaal epilsaktke |
| 181 | emasalvhil qstgkvkdfl tdlmmsevdr cgdnehlifr entlatkaie eylklvgqky |
| 241 | lqdalgefik alyesdence vdpskcsaad lpehqgnlkm ccelafckti nsycvfprel |
| 301 | kevfaswrqe cssrgrpdis erlisaslfl rflcpaimsp slfnllqeyp ddrtartltl |
| 361 | iakvtqnlan fakfgskeey msfmnqfleh ewtnmqrfll eisnpetlsn tagfegyidl |
| 421 | grelsslhsl lweavsqleq sivsklgplp rilrdvhtal stpgsgqlpg tndlastpgs |
| 481 | gsssisaglq kmviendlsg lidftrlpsp tpenkdlffv trssgvqpsp arsssysean |
| 541 | epdlqmangg kslsmvdlqd artidgeags pagpdvlptd gqaaaaqlva gwparatpvn |
| 601 | laglatvrra gqtpttpgts egapgrpqll aplsfqnpvy qmaaglplsp rglgdsgseg |
| 661 | hsslsshsns eelaaaaklg sfstaaeela rrpgelarrq msltekggqp tvprqnsagp |
| 721 | qrridqpspp ppppppaprg rtppnllstl qyprpssgtl asaspdwvgp strlrqqsss |
| 781 | skgdspelkp ravhkqgpsp vspnaldrta awlltmnaql ledeglgpdp phrdrlrskd |
| 841 | elsqaekdla vlqdklrist kkleeyetlf kcqeettqkl vleyqarlee geerlrrhee |
| 901 | dkdiqmkgii srlmsveeel kkdhaemqaa vdskqkiida qekriaslda anarlmsalt |
| 961 | qlkesmh |

IE. Nuclear Matrix Protein p84

Nuclear matrix protein p84 has been identified as an Akt substrate that interacts with Akt. Nuclear matrix protein p84 is associated with reference sequence NP_{—}005122 (GenBank Accession No. 4826882). Other relevant sequences include mouse THO complex 1 associated with reference sequence NP_{—}705780 (GenBank Accession No. 23956332).

NP_005122. nuclear matrix protein p84 [gi: 4826882]

| | |
|---|---|
| LOCUS | NP_005122 657 aa linear PRI 06-OCT-2003 |
| DEFINITION | nuclear matrix protein p84 [Homo sapiens]. |
| ACCESSION | NP_005122 |
| VERSION | NP_005122.1 GI: 4826882 |
| DBSOURCE | REFSEQ: accession NM_005131.1 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 657) |
| AUTHORS | Strasser, K., Masuda, S., Mason, P., Pfannstiel, J., Oppizzi, M., |
| | Rodriguez-Navarro, S., Rondon, A. G., Aguilera, A., Struhl, K., Reed, R. |
| | and Hurt, E. |
| TITLE | TREX is a conserved complex coupling transcription with messenger |
| | RNA export |
| JOURNAL | Nature 417 (6886), 304-308 (2002) |
| MEDLINE | 22010388 |
| PUBMED | 11979277 |
| REFERENCE | 2 (residues 1 to 657) |
| AUTHORS | Durfee, T., Mancini, M. A., Jones, D., Elledge, S. J. and Lee, W. H. |
| TITLE | The amino-terminal region of the retinoblastoma gene product binds |
| | a novel nuclear matrix protein that co-localizes to centers for RNA |
| | processing |
| JOURNAL | J. Cell Biol. 127 (3), 609-622 (1994) |
| MEDLINE | 95050936 |
| PUBMED | 7525595 |
| COMMENT | PROVISIONAL REFSEQ: This record has not yet been subject to final |
| | NCBI review. The reference sequence was derived from L36529.1. |
| | Summary: HPR1 is part of the TREX (transcription/export) complex, |
| | which includes TEX1 (MIM 606929), THO2 (MM 300395), ALY (MIM |
| | 604171), and UAP56 (MM 606390). [supplied by OMIM]. |
| FEATURES | Location/Qualifiers |
| source | 1 . . . 657 |
| | /organism=“Homo sapiens” |
| | /db_xref=“taxon:9606” |
| | /chromosome=“18” |
| | /map=“18p11.32” |
| Protein | 1 . . . 657 |
| | /product=“nuclear matrix protein p84” |
| Region | 561 . . . 652 |
| | /region_name=“DEATH domain, found in proteins involved in |
| | cell death (apoptosis). Alpha-helical domain present in a |
| | variety of proteins with apoptotic functions. Some (but |
| | not all) of these domains form homotypic and heterotypic |
| | dimers” |
| | /note=“DEATH” |
| | /db_xref=“CDD:smart00005” |
| variation | 561 |
| | /allele=“P” |
| | /allele=“L” |
| | /db_xref=“dbSNP:7343043” |
| variation | 653 |
| | /allele=“N” |
| | /allele=“H” |
| | /db_xref=“dbSNP:657541” |
| CDS | 1 . . . 657 |
| | /gene=“THOC1” |
| | /coded_by=“NM_005131.1:15..1988” |
| | /note=“go_component: nucleus [goid 0005634] [evidence TAS] |
| | [pmid 7525595]; |
| | go_process: RNA processing [goid 0006396] [evidence TAS] |
| | [pmid 7525595]; |
| | go_process: signal transduction [goid 0007165] [evidence |
| | IEA]” |
| | /db_xref=“GeneID:9984” |
| | /db_xref=“LocusID:9984” |
| | /db_xref=“MIM:606930” |

Origin

| [SEQ ID NO: 11] | |
|---|---|
| 1 | msptpplfsl peartrftks trealnnkni kpllstfsqv pgsenekkct ldqafrgile |
| 61 | eeiinhssce nvlaiislai ggvtegicta stpfvllgdv ldclpldqcd tiftfveknv |
| 121 | atwksntfya agknyllrmc ndllrrlsks qntvfcgriq lflarlfpls eksglnlqsq |
| 181 | fnlenvtvfn tneqestlgq khtedreegm dveegemgde eapttcsipi dynlyrkfws |
| 241 | lqdyfrnpvq cyekiswktf lkyseevlav fksyklddtq asrkkmeelk tggehvyfak |
| 301 | fltseklmdl qlsdsnfrrh illqylilfq ylkgqvkfks snyvltdeqs lwiedttksv |
| 361 | yqllsenppd gerfskmveh ilnteenwns wknegcpsfv kertsdtkpt riirkrtape |
| 421 | dflgkgptkk iltgneeltr lwnlcpdnme acksetrehm ptleeffeea ieqadpenma |
| 481 | eneykamnns nygwralkll arrsphffaj tnqqfkslqe ylenmvikla kelpppseei |
| 541 | ktgededeed ndallkenes pdvrrdkpvt geqievfank lgeqwkilap ylemkdseir |
| 601 | qiecdsedmk mrakqllvaw qdqegvhatp enlinainks glsdlaeslt ndnetns |

IF. HIRA Interacting Protein 3 (HIRIP3)

HIRA interacting protein 3 (HIRIP3) has been identified as an Akt substrate that interacts with Akt. This protein is associated with reference sequence NP_{—}003600 (GenBank Accession No. 21396500).

NP_003600. HIRA interacting protein 3 [21396500]

| | |
|---|---|
| LOCUS | NP_003600 556 aa linear PRI 04-OCT-2003 |
| DEFINITION | HIRA interacting protein 3; HIRA-interacting protein 3 [Homo |
| | sapiens]. |
| ACCESSION | NP_003600 |
| VERSION | NP_003600.2 GI: 21396500 |
| DBSOURCE | REFSEQ: accession NM_003609.2 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 556) |
| AUTHORS | Lorain, S., Quivy, J. P., Monier-Gavelle, F., Scamps, C., Lecluse, Y., |
| | Almouzni, G. and Lipinski, M. |
| TITLE | Core histones and HIRIP3, a novel histone-binding protein, directly |
| | interact with WD repeat protein HIRA |
| JOURNAL | Mol. Cell. Biol. 18 (9), 5546-5556 (1998) |
| MEDLINE | 98378566 |
| PUBMED | 9710638 |
| COMMENT | REVIEWED REFSEQ: This record has been curated by NCBI staff. The |
| | reference sequence was derived from AJ223351.2 and BC000588.2. |
| | On Jun 12, 2002 this sequence version replaced gi: 5803031. |
| | Summary: The HIRA protein shares sequence similarity with Hir1p |
| | and Hir2p, the two corepressors of histone gene transcription |
| | characterized in the yeast, Saccharomyces cerevisiae. The |
| | structural features of the HERA protein suggest that it may |
| | function as part of a multiprotein complex. Recently, several |
| | cDNAs encoding HIRA-interacting proteins, or HIRIPs, have been |
| | identified. In vitro, the HIRIP3 gene product binds HIRA, as well |
| | as H2B and H3 core histones, indicating that a complex containing |
| | HIRA-HIRIP3 could function in some aspects of chromatin and histone |
| | metabolism. |
| FEATURES | Location/Qualifiers |
| source | 1 . . . 556 |
| | /organism=“Homo sapiens” |
| | /db_xref=“taxon:9606” |
| | /chromosome=“16” |
| | /map=“16p12.1” |
| Protein | 1 . . . 556 |
| | /product=“HIRA interacting protein 3” |
| | /note=“HIRA-interacting protein 3” |
| CDS | 1 . . . 556 |
| | /gene=“HIRIP3” |
| | /coded_by=“NM_003609.2:462..2132” |
| | /note=“go_component: nucleus [goid 0005634] [evidence IEA] |
| | [pmid 9710638]; |
| | go_process: chromatin assembly/disassembly [goid 0006333] |
| | [evidence P] [pmid 9710638]” |
| | /db_xref=“GeneID:8479” |
| | /db_xref=“LocusID:8479” |
| | /db_xref=“MIM:603365” |

Origin

| [SEQ ID NO: 12] | |
|---|---|
| 1 | marekemqef trsffrgrpd lstlthsivr rrylahsgrs hlepeekqal krlveeellk |
| 61 | mqvdeaasre dkldltkkgk rpptpcsdpe rkrfrfnses esgseasspd yfgppakngv |
| 121 | aaevspakee nprraskave essdeerqrd lpaqrgeess eeeekgykgk trkkpvvkkq |
| 181 | apgkasvsrk qareeseese aepvqrtakk vegnkgtksl keseqeseee ilaqkkeqre |
| 241 | eeveeeekee deekgdwkpr trsngrrksa reersckqks qakrllgdsd seeeqkeaas |
| 301 | sgddsgrdre ppvqrksedr tqlkggkrls gssedeedsg kgeptakgsr kmarlgstsg |
| 361 | eesdlerevs dseagggpqg erknrsskks srkgrtrsss sssdgspeak ggkagsgrrg |
| 421 | edhpavmrlk ryiracgahr nykkllgscc shkerlsilr aelealgmkg tpslgkcral |
| 481 | keqreeaaev asidvanlis gsgrprrrta wnplgeaapp gelyrrtlds deerprpapp |
| 541 | dwshmrgiis sdgesn |

IG. HSP71

Heat shock 70 kDa protein 8 isoform 2 has been identified as an Akt substrate that interacts with Akt. This protein is associated with reference sequence NP_{—}694881 (GenBank Accession No. 24234686). In addition, Heat shock 70 kDa protein 8 isoform 1, associated with reference sequence NP_{—}006588 (GenBank Accession No. 5729877), is identified as an Akt substrate that interacts with Akt. A specific sequence identified as an Akt substrate includes heat shock protein HSP71 (GenBank Accession No. 20820471).

NP_694881. Heat shock 70 kDa protein 8 isoform 2 [gi: 24234686]

| | |
|---|---|
| LOCUS | NP_694881 493 aa linear PRI 05-OCT-2003 |
| DEFINITION | heat shock 70 kDa protein 8 isoform 2; heat shock cognate protein, |
| | 71-kDa; heat shock 70 kd protein 10; heat shock cognate protein 54; |
| | constitutive heat shock protein 70; lipopolysaccharide-associated |
| | protein 1; LPS-associated protein 1 [Homo sapiens]. |
| ACCESSION | NP_694881 |
| VERSION | NP_694881.1 GI: 24234686 |
| DBSOURCE | REFSEQ: accession NM_153201.1 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 493) |
| AUTHORS | Shin, B. K., Wang, H., Yim, A. M., Le Naour, F., Brichory, F., Jang, J. H., |
| | Zhao, R., Puravs, E., Tra, J., Michael, C. W., Misek, D. E. and |
| | Hanash, S. M. |
| TITLE | Global profiling of the cell surface proteome of cancer cells |
| | uncovers an abundance of proteins with chaperone function |
| JOURNAL | J. Biol. Chem. 278 (9), 7607-7616 (2003) |
| MEDLINE | 22486673 |
| PUBMED | 12493773 |
| REFERENCE | 2 (residues 1 to 493) |
| AUTHORS | Noessner, E., Gastpar, R., Milani, V., Brandl, A., Hutzler, P. J., |
| | Kuppner, M. C., Roos, M., Kremmer, E., Asea, A., Calderwood, S. K. and |
| | Issels, R. D. |
| TITLE | Tumor-derived heat shock protein 70 peptide complexes are |
| | cross-presented by human dendritic cells |
| JOURNAL | J. Immunol. 169 (10), 5424-5432 (2002) |
| MEDLINE | 22309089 |
| PUBMED | 12421917 |
| REMARK | GeneRIF: Tumor-derived HSP70 peptide complexes have the |
| | immunogenic |
| | potential to instruct dendritic cells to cross-present endogenously |
| | expressed, nonmutated, and tumor antigenic peptides shared among |
| | tumors of the melanocytic lineage for T cell recognition. |
| REFERENCE | 3 (residues 1 to 493) |
| AUTHORS | Triantafilou, K., Triantafilou, M. and Dedrick, R. L. |
| TITLE | A CD14-independent LPS receptor cluster |
| JOURNAL | Nat. Immunol. 2 (4), 338-345 (2001) |
| MEDLINE | 21174328 |
| PUBMED | 11276205 |
| REFERENCE | 4 (sites) |
| AUTHORS | Tsukahara, F., Yoshioka, T. and Muraki, T. |
| TITLE | Molecular and functional characterization of HSC54, a novel variant |
| | of human heat-shock cognate protein 70 |
| JOURNAL | Mol. Pharmacol. 58 (6), 1257-1263 (2000) |
| MEDLINE | 20545701 |
| PUBMED | 11093761 |
| REFERENCE | 5 (residues 1 to 493) |
| AUTHORS | Egerton, M., Moritz, R. L., Druker, B., Kelso, A. and Simpson, R. J. |
| TITLE | Identification of the 70 kD heat shock cognate protein (Hsc70) and |
| | alpha-actinin-1 as novel phosphotyrosine-containing proteins in T |
| | lymphocytes |
| JOURNAL | Biochem. Biophys. Res. Commun. 224 (3), 666-674 (1996) |
| MEDLINE | 96311348 |
| PUBMED | 8713105 |
| REFERENCE | 6 (residues 1 to 493) |
| AUTHORS | Tavaria, M., Gabriele, T., Anderson, R. L., Mirault, M. E., Baker, E., |
| | Sutherland, G. and Kola, I. |
| TITLE | Localization of the gene encoding the human heat shock cognate |
| | protein, HSP73, to chromosome 11 |
| JOURNAL | Genomics 29 (1), 266-268 (1995) |
| MEDLINE | 96079119 |
| PUBMED | 8530083 |
| REFERENCE | 7 (residues 1 to 493) |
| AUTHORS | Rensing, S. A. and Maier, U. G. |
| TITLE | Phylogenetic analysis of the stress-70 protein family |
| JOURNAL | J. Mol. Evol. 39 (1), 80-86 (1994) |
| MEDLINE | 94343547 |
| PUBMED | 7545947 |
| REFERENCE | 8 (residues 1 to 493) |
| AUTHORS | Dworniczak, B. and Mirault, M. E. |
| TITLE | Structure and expression of a human gene coding for a 71 kd heat |
| | shock ‘cognate’ protein |
| JOURNAL | Nucleic Acids Res. 15 (13), 5181-5197 (1987) |
| MEDLINE | 87259994 |
| PUBMED | 3037489 |
| COMMENT | REVIEWED REFSEO: This record has been curated by NCBI staff. The |
| | reference sequence was derived from AB034951.1, AK096100.1 and |
| | BC019816.2. |
| | Summary: The product encoded by this gene belongs to the heat shock |
| | protein 70 family which contains both heat-inducible and |
| | constitutively expressed members. The latter are called heat-shock |
| | cognate proteins. This gene encodes a heat-shock cognate protein. |
| | This protein binds to nascent polypeptides to facilitate correct |
| | folding. It also functions as an ATPase in the disassembly of |
| | clathrin-coated vesicles during transport of membrane components |
| | through the cell. Two alternatively spliced variants have been |
| | characterized to date. |
| | Transcript Variant: This variant (2) uses an alternate in-frame |
| | splice site in the 3′ coding region, compared to variant 1, |
| | resulting in a shorter protein (isoform 2). |
| FEATURES | Location/Qualifiers |
| source | 1 . . . 493 |
| | /organism=“Homo sapiens” |
| | /db_xref=“taxon:9606” |
| | /chromosome=“11” |
| | /map=“11q24.1” |
| Protein | 1 . . . 493 |
| | /product=“heat shock 70 kDa protein 8 isoform 2” |
| | /note=“heat shock cognate protein, 71-kDa; heat shock 70 kd |
| | protein 10; heat shock cognate protein 54; constitutive |
| | heat shock protein 70; lipopolysaccharide-associated |
| | protein 1; LPS-associated protein 1” |
| Region | 6 . . . 462 |
| | /region_name=“Hsp70 protein. Hsp70 chaperones help to fold |
| | many proteins. Hsp70 assisted folding involves repeated |
| | cycles of substrate binding and release. Hsp70 activity is |
| | ATP dependent. Hsp70 proteins are made up of two regions: |
| | the amino terminus is the ATPase domain and the carboxyl |
| | terminus is the substrate binding region” |
| | /note=“HSP70” |
| | /db_xref=“CDD:pfam00012” |
| CDS | 1 . . . 493 |
| | /gene=“HSPA8” |
| | /coded_by=“NM_153201.1:79..1560” |
| | /note=“go_component: intracellular [goid 0005622] |
| | [evidence NAS] [pmid 8713105]; |
| | go_function: ATP binding [goid 0005524] [evidence IEA]; |
| | go_function: heat shock protein activity [goid 0003773] |
| | [evidence IEA]; |
| | go_function: non-chaperonin molecular chaperone ATPase |
| | activity [goid 0008571] [evidence NAS] [pmid 8530083]; |
| | go_process: protein folding [goid 0006457] [evidence NAS] |
| | [pmid 8530083]” |
| | /db_xref=“GeneID:3312” |
| | /db_xref=“LocusID:3312” |
| | /db_xref=“MIM:600816” |

Origin

| [SEQ ID NO: 13] | |
|---|---|
| 1 | mskgpavgid lgttyscvgv fqhgkveiia ndqgnrttps yvaftdterl igdaaknqva |
| 61 | mnptntvfda krligrrfdd avvqsdmkhw pfmvvndagr pkvqveykge tksfypeevs |
| 121 | smvltkmkei aeaylgktvt navvtvpayf ndsqrqatkd agtiaglnvl riineptaaa |
| 181 | iaygldkkvg aernvlifdl gggtfdvsil tiedgifevk stagdthlgg edfdnrmvnh |
| 241 | fiaefkrkhk kdisenkrav rrlrtacera krtlssstqa sieidslyeg idfytsitra |
| 301 | rfeelnadlf rgtldpveka lrdakldksq ihdivlvggs tripkiqkll qdffngkeln |
| 361 | ksinpdeava ygaavqaail sgdksenvqd lllldvtpls lgietaggvm tvlikrntti |
| 421 | ptkqtqtftt ysdnqpgvli qvyegeramt kdnnllgkfe ltgmpggmpg gfpgggapps |
| 481 | ggassgptie evd |

NP_006588. heat shock 70 kDa protein 8 isoform 1 [gi: 5729877]

| | |
|---|---|
| LOCUS | NP_006588 646 aa linear PRI 05-OCT-2003 |
| DEFINITION | heat shock 70 kDa protein 8 isoform 1; heat shock cognate protein, |
| | 71-kDa; heat shock 70 kd protein 10; heat shock cognate protein 54; |
| | constitutive heat shock protein 70; lipopolysaccharide-associated |
| | protein 1; LPS-associated protein 1 [Homo sapiens]. |
| ACCESSION | NP_006588 |
| VERSION | NP_006588.1 GI: 5729877 |
| DBSOURCE | REFSEQ: accession NM_006597.3 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 646) |
| AUTHORS | Shin, B. K., Wang, H., Yim, A. M., Le Naour, F., Brichory, F., Jang, J. H., |
| | Zhao, R., Puravs, E., Tra, J., Michael, C. W., Misek, D. E. and |
| | Hanash, S. M. |
| TITLE | Global profiling of the cell surface proteome of cancer cells |
| | uncovers an abundance of proteins with chaperone function |
| JOURNAL | J. Biol. Chem. 278 (9), 7607-7616 (2003) |
| MEDLINE | 22486673 |
| PUBMED | 12493773 |
| REFERENCE | 2 (residues 1 to 646) |
| AUTHORS | Noessner, E., Gastpar, R., Milani, V., Brandl, A., Hutzler, P. J., |
| | Kuppner, M. C., Roos, M., Kremmer, E., Asea, A., Calderwood, S. K. and |
| | Issels, R. D. |
| TITLE | Tumor-derived heat shock protein 70 peptide complexes are |
| | cross-presented by human dendritic cells |
| JOURNAL | J. Immunol. 169 (10), 5424-5432 (2002) |
| MEDLINE | 22309089 |
| PUBMED | 12421917 |
| REMARK | GeneRIF: Tumor-derived HSP70 peptide complexes have the |
| | immunogenic potential to instruct dendritic cells to cross-present endogenously |
| | expressed, nonmutated, and tumor antigenic peptides shared among |
| | tumors of the melanocytic lineage for T cell recognition. |
| REFERENCE | 3 (residues 1 to 646) |
| AUTHORS | Triantafilou, K., Triantafilou, M. and Dedrick, R. L. |
| TITLE | A CD14-independent LPS receptor cluster |
| JOURNAL | Nat. Immunol. 2 (4), 338-345 (2001) |
| MEDLINE | 21174328 |
| PUBMED | 11276205 |
| REFERENCE | 4 (residues 1 to 646) |
| AUTHORS | Tsukahara, F., Yoshioka, T. and Muraki, T. |
| TITLE | Molecular and functional characterization of HSC54, a novel variant |
| | of human heat-shock cognate protein 70 |
| JOURNAL | Mol. Pharmacol. 58 (6), 1257-1263 (2000) |
| MEDLINE | 20545701 |
| PUBMED | 11093761 |
| REFERENCE | 5 (residues 1 to 646) |
| AUTHORS | Egerton, M., Moritz, R. L., Druker, B., Kelso, A. and Simpson, R. J. |
| TITLE | Identification of the 70 kD heat shock cognate protein (Hsc70) and |
| | alpha-actinin-1 as novel phosphotyrosine-containing proteins in T |
| | lymphocytes |
| JOURNAL | Biochem. Biophys. Res. Commun. 224 (3), 666-674 (1996) |
| MEDLINE | 96311348 |
| PUBMED | 8713105 |
| REFERENCE | 6 (residues 1 to 646) |
| AUTHORS | Tavaria, M., Gabriele, T., Anderson, R. L., Mirault, M. E., Baker, E., |
| | Sutherland, G. and Kola, I. |
| TITLE | Localization of the gene encoding the human heat shock cognate |
| | protein, HSP73, to chromosome 11 |
| JOURNAL | Genomics 29 (1), 266-268 (1995) |
| MEDLINE | 96079119 |
| PUBMED | 8530083 |
| REFERENCE | 7 (residues 1 to 646) |
| AUTHORS | Rensing, S. A. and Maier, U. G. |
| TITLE | Phylogenetic analysis of the stress-70 protein family |
| JOURNAL | J. Mol. Evol. 39 (1), 80-86 (1994) |
| MEDLINE | 94343547 |
| PUBMED | 7545947 |
| REFERENCE | 8 (residues 1 to 646) |
| AUTHORS | Dworniczak, B. and Mirault, M. E. |
| TITLE | Structure and expression of a human gene coding for a 71 kd heat |
| | shock ‘cognate’ protein |
| JOURNAL | Nucleic Acids Res. 15 (13), 5181-5197 (1987) |
| MEDLINE | 87259994 |
| PUBMED | 3037489 |
| COMMENT | REVIEWED REFSEQ: This record has been curated by NCBI staff. The |
| | reference sequence was derived from BC019816.2 and AK096100.1. |
| | Summary: The product encoded by this gene belongs to the heat shock |
| | protein 70 family which contains both heat-inducible and |
| | constitutively expressed members. The latter are called heat-shock |
| | cognate proteins. This gene encodes a heat-shock cognate protein. |
| | This protein binds to nascent polypeptides to facilitate correct |
| | folding. It also functions as an ATPase in the disassembly of |
| | clathrin-coated vesicles during transport of membrane components |
| | through the cell. Two alternatively spliced variants have been |
| | characterized to date. |
| | Transcript Variant: This variant (1) represents the longer |
| | transcript and encodes the longer isoform. |
| FEATURES | Location/Qualifiers |
| source | 1 . . . 646 |
| | /organism=“Homo sapiens” |
| | /db_xref=“taxon:9606” |
| | /chromosome=“11” |
| | /map=“11q24.1” |
| Protein | 1 . . . 646 |
| | /product=“heat shock 70 kDa protein 8 isoform 1” |
| | /note=“heat shock cognate protein, 71-kDa; heat shock 70 kd |
| | protein 10; heat shock cognate protein 54; constitutive |
| | heat shock protein 70; lipopolysaccharide-associated |
| | protein 1; LPS-associated protein 1” |
| Region | 6 . . . 612 |
| | /region_name=“Hsp70 protein. Hsp70 chaperones help to fold |
| | many proteins. Hsp70 assisted folding involves repeated |
| | cycles of substrate binding and release. Hsp70 activity is |
| | ATP dependent. Hsp70 proteins are made up of two regions: |
| | the amino terminus is the ATPase domain and the carboxyl |
| | terminus is the substrate binding region” |
| | /note=“HSP70” |
| | /db_xref=“CDD:pfam00012” |
| CDS | 1 . . . 646 |
| | /gene=“HSPA8” |
| | /coded_by=“NM_006597.3:79..2019” |
| | /note=“go_component: intracellular [goid 0005622] |
| | [evidence NAS] [pmid 8713105]; |
| | go_function: ATP binding [goid 0005524] [evidence IEA]; |
| | go_function: heat shock protein activity [goid 0003773] |
| | [evidence IEA]; |
| | go_function: non-chaperonin molecular chaperone ATPase |
| | activity [goid 0008571] [evidence NAS] [pmid 8530083]; |
| | go_process: protein folding [goid 0006457] [evidence NAS] |
| | [pmid 8530083]” |
| | /db_xref=“GeneID:3312” |
| | /db_xref=“LocusID:3312” |
| | /db_xref=“MIM:600816” |

Origin

| [SEQ ID NO: 14] | | | | | | |
|---|---|---|---|---|---|---|
| 1 | mskgpavgid | lgttyscvgv | fqhgkveiia | ndqgnrttps | yvaftdterl | igdaaknqva |
| 61 | mnptntvfda | krligrrfdd | avvqsdmkhw | pfmvvndagr | pkvqveykge | tksfypeevs |
| 121 | smvltkmkei | aeaylgktvt | navvtvpayf | ndsqrqatkd | agtiaglnvl | riineptaaa |
| 181 | iaygldkkvg | aernvlifdl | gggtfdvsil | tiedgifevk | stagdthlgg | edfdnrmvnh |
| 241 | fiaefkrkhk | kdisenkrav | rrlrtacera | krtlssstqa | sieidslyeg | idfytsitra |
| 301 | rfeelnadlf | rgtldpveka | lrdakldksq | ihdivlvggs | tripkiqkll | qdffngkeln |
| 361 | ksinpdeava | ygaavqaail | sgdksenvqd | lllldvtpls | lgietaggvm | tvlikrntti |
| 421 | ptkqtqtftt | ysdnqpgvli | qvyegeramt | kdnnllgkfe | ltgippaprg | vpqievtfdi |
| 481 | dangilnvsa | vdkstgkenk | ititndkgrl | skediermvq | eaekykaede | kqrdkvsskn |
| 541 | slesyafnmk | atvedeklqg | kindedkqki | ldkcneiinw | ldknqtaeke | efehqqkele |
| 601 | kvcnpiitkl | yqsaggmpgg | mpggfpggga | ppsggassgp | tieevd | |

IH. Ribosomal Protein L6

Ribosomal protein L6 has been identified as an Akt substrate that interacts with Akt. This protein is associated with reference sequence NP_{—}000961 (GenBank Accession No. 16753227). Other relevant sequences include rat ribosomal protein L6 associated with reference sequence NP_{—}446423 (GenBank Accession No. 16758864).

NP_000961. Ribosomal protein L6 [gi: 16753227]

| | |
|---|---|
| LOCUS | NP_000961 288 aa linear PRI 05-OCT-2003 |
| DEFINITION | ribosomal protein L6; 60S ribosomal protein L6; tax-responsive |
| | enhancer element-binding protein 107; DNA-binding protein |
| | TAXREB107; neoplasm-related protein C140 [Homo sapiens]. |
| ACCESSION | NP_000961 |
| VERSION | NP_000961.2 GI: 16753227 |
| DBSOURCE | REFSEQ: accession NM_000970.2 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 288) |
| AUTHORS | Du, J. P., Jin, X. H., Shi, Y. Q., Cao, Y. X., Zhao, Y. Q., Liu, C. J., Yin, F., |
| | Hu, W. H., Chen, B. J., Qiao, T. D. and Fan, D. M. |
| TITLE | Differential expression of RPL6/Taxreb107 in drug resistant gastric |
| | cancer cell line SGC7901/ADR and its correlation with multiple-drug |
| | resistance |
| JOURNAL | Zhonghua Zhong Liu Za Zhi 25 (1), 21-25 (2003) |
| MEDLINE | 22566791 |
| PUBMED | 12678981 |
| REMARK | GeneRIF: The high expression of RPL6/Taxreb107 in drug resistant |
| | gastric cancer cell shows its correlation with multiple-drug |
| | resistance in gastric cancer. |
| REFERENCE | 2 (residues 1 to 288) |
| AUTHORS | Uechi, T., Tanaka, T. and Kenmochi, N. |
| TITLE | A complete map of the human ribosomal protein genes: assignment of |
| | 80 genes to the cytogenetic map and implications for human |
| | disorders |
| JOURNAL | Genomics 72 (3), 223-230 (2001) |
| MEDLINE | 21295043 |
| PUBMED | 11401437 |
| REFERENCE | 3 (residues 1 to 288) |
| AUTHORS | Kenmochi, N., Yoshihama, M., Higa, S. and Tanaka, T. |
| TITLE | The human ribosomal protein L6 gene in a critical region for Noonan |
| | syndrome |
| JOURNAL | J. Hum. Genet. 45 (5), 290-293 (2000) |
| MEDLINE | 20496216 |
| PUBMED | 11043511 |
| REFERENCE | 4 (residues 1 to 288) |
| AUTHORS | Ye, Z. and Connor, J. R. |
| TITLE | cDNA cloning by amplification of circularized first strand cDNAs |
| | reveals non-IRE-regulated iron-responsive mRNAs |
| JOURNAL | Biochem. Biophys. Res. Commun. 275 (1), 223-227 (2000) |
| MEDLINE | 20403900 |
| PUBMED | 10944468 |
| REFERENCE | 5 (residues 1 to 288) |
| AUTHORS | Kenmochi, N., Kawaguchi, T., Rozen, S., Davis, E., Goodman, N., |
| | Hudson, T. J., Tanaka, T. and Page, D. C. |
| TITLE | A map of 75 human ribosomal protein genes |
| JOURNAL | Genome Res. 8 (5), 509-523 (1998) |
| MEDLINE | 98248690 |
| PUBMED | 9582194 |
| REFERENCE | 6 (residues 1 to 288) |
| AUTHORS | Monk, S., Sakuntabhai, A., Carter, S. A., Bryce, S. D., Cox, R., |
| | Harrington, L., Levy, E., Ruiz-Perez, V. L., Katsantoni, E., |
| | Kodvawala, A., Munro, C. S., Burge, S., Larregue, M., Nagy, G., |
| | Rees, J. L., Lathrop, M., Monaco, A. P., Strachan, T. and Hovnanian, A. |
| TITLE | Refined genetic mapping of the darier locus to a <1-cM region of |
| | chromosome 12q24.1, and construction of a complete, high-resolution |
| | P1 artificial chromosome/bacterial artificial chromosome contig of |
| | the critical region |
| JOURNAL | Am. J. Hum. Genet. 62 (4), 890-903 (1998) |
| MEDLINE | 98198349 |
| PUBMED | 9529352 |
| REFERENCE | 7 (residues 1 to 288) |
| AUTHORS | Wool, I. G., Chan, Y. L. and Gluck, A. |
| TITLE | Structure and evolution of mammalian ribosomal proteins |
| JOURNAL | Biochem. Cell Biol. 73 (11-12), 933-947 (1995) |
| MEDLINE | 96282697 |
| PUBMED | 8722009 |
| REMARK | This review focuses primarily on rat ribosomal proteins, but it |
| | compares them to human ribosomal proteins. |
| REFERENCE | 8 (residues 1 to 288) |
| AUTHORS | Ohta, K., Endo, T., Gunji, K. and Onaya, T. |
| TITLE | Isolation of a cDNA whose expression is markedly increased in |
| | malignantly transformed FRTL cells and neoplastic human thyroid |
| | tissues |
| JOURNAL | J. Mol. Endocrinol. 12 (1), 85-92 (1994) |
| MEDLINE | 94242236 |
| PUBMED | 8185817 |
| REFERENCE | 9 (residues 1 to 288) |
| AUTHORS | Zaman, G. J. |
| TITLE | Sequence of a cDNA encoding human ribosomal protein L26 and of a |
| | cDNA probably encoding human ribosomal protein L6 |
| JOURNAL | Nucleic Acids Res. 21 (7), 1673 (1993) |
| MEDLINE | 93241958 |
| PUBMED | 8479925 |
| REFERENCE | 10 (residues 1 to 288) |
| AUTHORS | Morita, T., Sato, T., Nyunoya, H., Tsujimoto, A., Takahara, J., Irino, S. |
| | and Shimotohno, K. |
| TITLE | Isolation of a cDNA clone encoding DNA-binding protein (TAXREB107) |
| | that binds specifically to domain C of the tax-responsive enhancer |
| | element in the long terminal repeat of human T-cell leukemia virus |
| | type I |
| JOURNAL | AIDS Res. Hum. Retroviruses 9 (2), 115-121 (1993) |
| MEDLINE | 93207816 |
| PUBMED | 8457378 |
| COMMENT | REVIEWED REFSEQ: This record has been curated by NCBI staff. The |
| | reference sequence was derived from BC004138.2. |
| | On Nov 6, 2001 this sequence version replaced gi: 4506657. |
| | Summary: Ribosomes, the organelles that catalyze protein synthesis, |
| | consist of a small 40S subunit and a large 60S subunit. Together |
| | these subunits are composed of 4 RNA species and approximately 80 |
| | structurally distinct proteins. This gene encodes a ribosomal |
| | protein that is a component of the 60S subunit. The protein belongs |
| | to the L6E family of ribosomal proteins. It is located in the |
| | cytoplasm. The protein can bind specifically to domain C of the |
| | tax-responsive enhancer element of human T-cell leukemia virus type |
| | 1, and it has been suggested that the protein may participate in |
| | tax-mediated transactivation of transcription. As is typical for |
| | genes encoding ribosomal proteins, there are multiple processed |
| | pseudogenes of this gene dispersed through the genome. |
| FEATURES | Location/Qualifiers |
| source | 1 . . . 288 |
| | /organism=“Homo sapiens” |
| | /db_xref=“taxon:9606” |
| | /chromosome=“12” |
| | /map=“12q24.1” |
| Protein | 1 . . . 288 |
| | /product=“ribosomal protein L6” |
| | /note=“60S ribosomal protein L6; tax-responsive enhancer |
| | element-binding protein 107; DNA-binding protein |
| | TAXREB107; neoplasm-related protein C140” |
| Region | 42 . . . 72 |
| | /region_name=“Ribosomal protein L6, N-terminal domain” |
| | /note=“Ribosomal_L6e_N” |
| | /db_xref=“CDD:pfam03868” |
| variation | 123 |
| | /allele=“P” |
| | /allele=“R” |
| | /db_xref=“dbSNP:3933539” |
| Region | 181 . . . 288 |
| | /region_name=“Ribosomal protein L6e” |
| | /note=“Ribosomal_L6e” |
| | /db_xref=“CDD:pfam01159” |
| variation | 245 |
| | /allele=“Q” |
| | /allele=“R” |
| | /db_xref=“dbSNP:15146” |
| CDS | 1 . . . 288 |
| | /gene=“RPL6” |
| | /coded_by=“NM_000970.2:32..898” |
| | /note=“go_component: cytosolic large ribosomal subunit |
| | (sensu Eukarya) [goid 0005842] [evidence TAS] [pmid |
| | 8479925]; |
| | go_component: ribosome [goid 0005840] [evidence IEA]; |
| | go_component: intracellular [goid 0005622] [evidence IEA]; |
| | go_function: structural protein of ribosome [goid 0003735] |
| | [evidence P] [pmid 8479925]; |
| | go_function: RNA binding [goid 0003723] [evidence TAS] |
| | [pmid 8479925]; |
| | go_function: DNA binding [goid 0003677] [evidence TAS] |
| | [pmid 8457378]; |
| | go_function: structural constituent of ribosome [goid |
| | 0003735] [evidence TAS] [pmid 8479925]; |
| | go_process: regulation of transcription, DNA-dependent |
| | [goid 0006355] [evidence TAS] [pmid 8457378]; |
| | go_process: protein biosynthesis [goid 0006412] [evidence |
| | TAS] [pmid 8479925]” |
| | /db_xref=“GeneID:6128” |
| | /db_xref=“LocusID:6128” |
| | /db_xref=“MIM:603703” |

Origin

| [SEQ ID NO: 15] | | | | | | |
|---|---|---|---|---|---|---|
| 1 | magekvekpd | tkekkpeakk | vdaggkvkkg | nlkakkpkkg | kphcsrnpvl | vrgigrysrs |
| 61 | amysrkamyk | rkysaakskv | ekkkkekvla | tvtkpvggdk | nggtrvvklr | kmpryypted |
| 121 | vprkllshgk | kpfsqhvrkl | rasitpgtil | iiltgrhrgk | rvvflkqlas | glllvtgplv |
| 181 | lnrvplrrth | qkfviatstk | idisnvkipk | hltdayfkkk | klrkprhqeg | eifdtekeky |
| 241 | eiteqrkidq | kavdsqilpk | ikaipqlqgy | lrsvfaltng | iyphklvf | |

II. Guanine Nucleotide Exchange Factor Lbc (GEF Lbc)

A-kinase anchor protein 13 isoform 2 (A-kinase anchoring protein; guanine nucleotide exchange factor Lbc (GEF Lbc) has been identified as an Akt substrate that interacts with Akt. This protein is associated with reference sequence NP_{—}009131 (GenBank Accession No. 21493029). In addition, A-kinase anchor protein 13 isoform 1, associated with reference sequence NP_{—}006729 (GenBank Accession No. 31563330), and A-kinase anchor protein 13 isoform 3, associated with reference sequence NP_{—}658913 (GenBank Accession No. 21493031), are identified as Akt substrates that interact with Akt. A specific sequence identified as an Akt substrate includes Guanine Nucleotide Exchange Factor Lbc (GEF Lbc) (GenBank Accession No. 15207794).

NP_009131. A-kinase anchor protein 13 isoform 2 [gi: 21493029]

| | |
|---|---|
| LOCUS | NP_009131 2813 aa linear PRI 05-OCT-2003 |
| DEFINITION | A-kinase anchor protein 13 isoform 2; A-kinase anchoring protein; |
| | guanine nucleotide exchange factor Lbc; breast cancer nuclear |
| | receptor-binding auxiliary protein; lymphoid blast crisis oncogene |
| | [Homo sapiens]. |
| ACCESSION | NP_009131 |
| VERSION | NP_009131.2 GI: 21493029 |
| DBSOURCE | REFSEQ: accession NM_007200.3 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 2813) |
| AUTHORS | Spierings, E., Brickner, A. G., Caldwell, J. A., Zegveld, S., Tatsis, N., |
| | Blokland, E., Pool, J., Pierce, R. A., Mollah, S., Shabanowitz, J., |
| | Eisenlohr, L. C., van Veelen, P., Ossendorp, F., Hunt, D. F., Goulmy, E. |
| | and Engelhard, V. H. |
| TITLE | The minor histocompatibility antigen HA-3 arises from differential |
| | proteasome-mediated cleavage of the lymphoid blast crisis (Lbc) |
| | oncoprotein |
| JOURNAL | Blood 102 (2), 621-629 (2003) |
| MEDLINE | 22718699 |
| PUBMED | 12663445 |
| REMARK | GeneRIF: The HA-3 peptide, VTEPGTAQY, is encoded by the lymphoid |
| | blast crisis oncogene, showing for the 1st time that a |
| | leukemia-associated oncogene can give rise to immunogenic T-cell |
| | epitopes that may participate in antihost & antileukemic alloimmune |
| | responses. |
| REFERENCE | 2 (residues 1 to 2813) |
| AUTHORS | Park, B., Nguyen, N. T., Dutt, P., Merdek, K. D., Bashar, M., |
| | Sterpetti, P., Tosolini, A., Testa, J. R. and Toksoz, D. |
| TITLE | Association of Lbc Rho guanine nucleotide exchange factor with |
| | alpha-catenin-related protein, alpha-catulin/CTNNAL1, supports |
| | serum response factor activation |
| JOURNAL | J. Biol. Chem. 277 (47), 45361-45370 (2002) |
| MEDLINE | 22323310 |
| PUBMED | 12270917 |
| REMARK | GeneRIF: Results show that alpha-catulin co-expression leads to |
| | increased Lbc-induced serum response factor activation and may |
| | modulate Rho pathway signaling in vivo by providing a scaffold for |
| | the Lbc Rho guanine nucleotide exchange factor. |
| REFERENCE | 3 (residues 1 to 2813) |
| AUTHORS | Diviani, D., Soderling, J. and Scott, J. D. |
| TITLE | AKAP-Lbc anchors protein kinase A and nucleates Galpha 12-selective |
| | Rho-mediated stress fiber formation |
| JOURNAL | J. Biol. Chem. 276 (47), 44247-44257 (2001) |
| MEDLINE | 21570178 |
| PUBMED | 11546812 |
| REFERENCE | 4 (residues 1 to 2813) |
| AUTHORS | Klussmann, E., Edemir, B., Pepperle, B., Tamma, G., Henn, V., |
| | Klauschenz, E., Hundsrucker, C., Maric, K. and Rosenthal, W. |
| TITLE | Ht31: the first protein kinase A anchoring protein to integrate |
| | protein kinase A and Rho signaling |
| JOURNAL | FEBS Lett. 507 (3), 264-268 (2001) |
| MEDLINE | 21553155 |
| PUBMED | 11696353 |
| REFERENCE | 5 (residues 1 to 2813) |
| AUTHORS | Sterpetti, P., Hack, A. A., Bashar, M. P., Park, B., Cheng, S. D., |
| | Knoll, J. H., Urano, T., Feig, L. A. and Toksoz, D. |
| TITLE | Activation of the Lbc Rho exchange factor proto-oncogene by |
| | truncation of an extended C terminus that regulates transformation |
| | and targeting |
| JOURNAL | Mol. Cell. Biol. 19 (2), 1334-1345 (1999) |
| MEDLINE | 99108106 |
| PUBMED | 9891067 |
| REFERENCE | 6 (residues 1 to 2813) |
| AUTHORS | Rubino, D., Driggers, P., Arbit, D., Kemp, L., Miller, B., Coso, O., |
| | Pagliai, K., Gray, K., Gutkind, S. and Segars, J. |
| TITLE | Characterization of Brx, a novel Dbl family member that modulates |
| | estrogen receptor action |
| JOURNAL | Oncogene 16 (19), 2513-2526 (1998) |
| MEDLINE | 98288806 |
| PUBMED | 9627117 |
| REFERENCE | 7 (residues 1 to 2813) |
| AUTHORS | Toksoz, D. and Williams, D. A. |
| TITLE | Novel human oncogene lbc detected by transfection with distinct |
| | homology regions to signal transduction products |
| JOURNAL | Oncogene 9 (2), 621-628 (1994) |
| MEDLINE | 94119604 |
| PUBMED | 8290273 |
| REFERENCE | 8 (residues 1 to 2813) |
| AUTHORS | Carr, D. W., Hausken, Z. E., Fraser, I. D., Stofko-Hahn, R. E. and |
| | Scott, J. D. |
| TITLE | Association of the type II cAMP-dependent protein kinase with a |
| | human thyroid RII-anchoring protein. Cloning and characterization |
| | of the RII-binding domain |
| JOURNAL | J. Biol. Chem. 267 (19), 13376-13382 (1992) |
| MEDLINE | 92317056 |
| PUBMED | 1618839 |
| REFERENCE | 9 (residues 1 to 2813) |
| AUTHORS | Carr, D. W., Stofko-Hahn, R. E., Fraser, I. D., Bishop, S. M., Acott, T. S., |
| | Brennan, R. G. and Scott, J. D. |
| TITLE | Interaction of the regulatory subunit (RII) of cAMP-dependent |
| | protein kinase with RII-anchoring proteins occurs through an |
| | amphipathic helix binding motif |
| JOURNAL | J. Biol. Chem. 266 (22), 14188-14192 (1991) |
| MEDLINE | 91317762 |
| PUBMED | 1860836 |
| COMMENT | REVIEWED REFSEQ: This record has been curated by NCBI staff. The |
| | reference sequence was derived from AB055890.1, AK091210.1, |
| | AL135297.1 and BC050312.1. |
| | On Jun 20, 2002 this sequence version replaced gi: 17933492. |
| | Summary: The A-kinase anchor proteins (AKAPs) are a group of |
| | structurally diverse proteins, which have the common function of |
| | binding to the regulatory subunit of protein kinase A (PKA) and |
| | confining the holoenzyme to discrete locations within the cell. |
| | This gene encodes a member of the AKAP family. Alternative splicing |
| | of this gene results in at least 3 transcript variants encoding |
| | different isoforms containing a dbl oncogene homology (DH) domain |
| | and a pleckstrin homology (PH) domain. The DH domain is associated |
| | with guanine nucleotide exchange activation for the Rho/Rac family |
| | of small GTP binding proteins, resulting in the conversion of the |
| | inactive GTPase to the active form capable of transducing signals. |
| | The PH domain has multiple functions. Therefore, these isoforms |
| | function as scaffolding proteins to coordinate a Rho signaling |
| | pathway and, in addition, function as protein kinase A-anchoring |
| | proteins. |
| | Transcript Variant: This variant (2) contains an internal alternate |
| | in-frame exon in the coding region, as compared to variant 1. |
| | Isoform 2 differs from isoform 1 in a small middle region. |
| FEATURES | Location/Qualifiers |
| source | 1 . . . 2813 |
| | /organism=“Homo sapiens” |
| | /db_xref=“taxon:9606” |
| | /chromosome=“15” |
| | /map=“15q24-q25” |
| Protein | 1 . . . 2813 |
| | /product=“A-kinase anchor protein 13 isoform 2” |
| | /note=“A-kinase anchoring protein; guanine nucleotide |
| | exchange factor Lbc; breast cancer nuclear |
| | receptor-binding auxiliary protein; lymphoid blast crisis |
| | oncogene” |
| Region | 166 . . . 224 |
| | /region_name=“ankyrin repeats” |
| variation | 452 |
| | /allele=“T” |
| | /allele=“M” |
| | /db_xref=“dbSNP:2061821” |
| variation | 494 |
| | /allele=“W” |
| | /allele=“R” |
| | /db_xref=“dbSNP:2061822” |
| variation | 574 |
| | /allele=“R” |
| | /allele=“C” |
| | /db_xref=“dbSNP:2061824” |
| variation | 624 |
| | /allele=“V” |
| | /allele=“G” |
| | /db_xref=“dbSNP:745191” |
| variation | 689 |
| | /allele=“K” |
| | /allele=“E” |
| | /db_xref=“dbSNP:7177107” |
| variation | 845 |
| | /allele=“V” |
| | /allele=“A” |
| | /db_xref=“dbSNP:4075256” |
| variation | 897 |
| | /allele=“V” |
| | /allele=“M” |
| | /db_xref=“dbSNP:4075254” |
| variation | 1062 |
| | /allele=“P” |
| | /allele=“A” |
| | /db_xref=“dbSNP:4843074” |
| variation | 1086 |
| | /allele=“N” |
| | /allele=“D” |
| | /db_xref=“dbSNP:4843075” |
| variation | 1216 |
| | /allele=“T” |
| | /allele=“M” |
| | /db_xref=“dbSNP:7162168” |
| Region | 1788 . . . 1826 |
| | /region name=“C1 homology region” |
| Region | 1998 . . . 2189 |
| | /region_name=“Guanine nucleotide exchange factor for |
| | Rho/Rac/Cdc42-like GTPases” |
| | /note=“RhoGEF” |
| | /db_xref=“CDD:smart00325” |
| variation | 2179 |
| | /allele=“N” |
| | /allele=“S” |
| | /db_xref=“dbSNP:3743323” |
| variation | 2457 |
| | /allele=“G” |
| | /allele=“S” |
| | /db_xref=“dbSNP:2241268” |
| variation | 2801 |
| | /allele=“A” |
| | /allele=“T” |
| | /db_xref=“dbSNP:2614668” |
| CDS | 1 . . . 2813 |
| | /gene=“AKAP13” |
| | /coded_by=“NM_007200.3:171..8612” |
| | /note=“go component: membrane fraction [goid 0005624] |
| | [evidence E]; |
| | go function: protein kinase A anchoring activity [goid |
| | 0005079] [evidence E] [pmid 1618839]; |
| | go_function: protein binding [goid 0005515] [evidence E] |
| | [pmid 1860836]; |
| | go_function: Rho guanyl-nucleotide exchange factor |
| | activity [goid 0005089] [evidence NR]; |
| | go_function: signal transducer activity [goid 0004871] |
| | [evidence P]; |
| | go_process: oncogenesis [goid 0007048] [evidence P]; |
| | go_process: signal transduction [goid 0007165] [evidence |
| | NR]” |
| | /db_xref=“GeneID:11214” |
| | /db_xref=“LocusID:11214” |
| | /db_xref=“MIM:604686” |

Origin

| [SEQ ID NO: 16] | | | | | | |
|---|---|---|---|---|---|---|
| 1 | mklnpqqapl | ygdcvvtvll | aeedkaeddv | vfylvflgst | lrhctstrkv | ssdtletiap |
| 61 | ghdccetvkv | qicaskegip | vfvvaeedfh | fvqdeaydaa | qflatsagnq | qalnftrfld |
| 121 | qsgppsgdvn | sldkklvlaf | rhikiptewn | vlgtdqslhd | agpretlmhf | avrlgllrlt |
| 181 | wfllqkpggr | galsihnqeg | atpvslaler | gyhklhqllt | eenagepdsw | sslsyeipyg |
| 241 | dcsvrhhrel | diytltsesd | shhehpfpgd | gctgpifklm | niqqqlmktn | lkqmdslmpl |
| 301 | mmtaqdpssa | petdgqflpc | apeptdpqrl | ssseetestq | ccpgspvaqt | espcdlssiv |
| 361 | eeentdrscr | kknkgverkg | eevepapivd | sgtvsdqdsc | lqslpdcgvk | gteglsscgn |
| 421 | rneetgtkss | gmptdqesls | sgdavlqrdl | vmepgtaqys | sggelggist | tnvstpdtag |
| 481 | emehglmnpd | atvwknvlqg | gestkerfen | snigtagasd | vhvtskpvdk | isvpncapaa |
| 541 | ssldgnkpae | sslafsneet | stektaetet | srsreesada | pvdqnsvvip | aaakdkisdg |
| 601 | lepytllaag | igeamspsdl | allgleedvm | phqnsetnss | haqsqkgkss | picsttgddk |
| 661 | lcadsacqqn | tvtssgdlva | klcdnivses | esttarqpss | qdppdashce | dpqahtvtsd |
| 721 | pvrdtqerad | fcpfkvvdnk | gqrkdvkldk | pltnmlevvs | hphpvvpkme | kelvpdqavi |
| 781 | sdstfslans | pgsesvtkdd | alsfvpsqke | kgtatpelht | atdyrdgpdg | nsnepdtrpl |
| 841 | edravglsts | staaelqhgm | gntsltglgg | ehegpappai | pealnikgnt | dsslqsvgka |
| 901 | tlaldsvlte | egkllvvses | saaqeqdkdk | avtcssiken | alssgtlqee | qrtpppgqdt |
| 961 | qqfheksisa | dcakdkalql | snspgassaf | lkaetehnke | vapqvslltq | ggaaqslvpp |
| 1021 | gaslatesrq | ealgaehnss | allpcllpdg | sdgsdalncs | qpspldvgvk | ntqsqgktsa |
| 1081 | cevsgdvtvd | vtgvnalqgm | aeprrenish | ntqdilipnv | llsqeknavl | glpvalqdka |
| 1141 | vtdpqgvgtp | emipldwekg | klegadhsct | mgdaeeaqid | deahpvllqp | vakelptdme |
| 1201 | lsahddgapa | gvrevmrapp | sgrerstpsl | pcmvsaqdap | lpkgadliee | aasrivdavi |
| 1261 | eqvkaagall | tegeachmsl | sspelgpltk | glesaftekv | stfppgeslp | mgstpeeatg |
| 1321 | slagcfagre | epekiilpvq | gpepaaempd | vkaedevdfr | assiseevav | gsiaatlkmk |
| 1381 | qgpmtqainr | enwctiepcp | daasllaskq | specenfldv | glgrectskq | gvlkresgsd |
| 1441 | sdlfhspsdd | mdsiifpkpe | eehlacditg | sssstddtas | ldrhsshgsd | vslsqilkpn |
| 1501 | rsrdrqsldg | fyshgmgaeg | resesepadp | gdveeeemds | itevpancsv | lrssmrslsp |
| 1561 | frrhswgpgk | naasdaemnh | rssmrvlgdv | vrrppihrrs | fslegltgga | gvgnkpsssl |
| 1621 | evssanaeel | rhpfsgeerv | dslvslseed | lesdqrehrm | fdqqichrsk | qqgfnyctsa |
| 1681 | isspltksis | lmtishpgld | nsrpfhstfh | ntsanltesi | teenynfiph | spskkdsewk |
| 1741 | sgtkvsrtfs | yiknkmsssk | kskekekekd | kikekekdsk | dkekdkktvn | ghtfssipvv |
| 1801 | gpiscsqcmk | pftnkdaytc | ancsafvhkg | creslascak | vkmkqpkgsl | qahdtsslpt |
| 1861 | vimrnkpsqp | kerprsavll | vdetattpif | anrrsqqsvs | lsksvsiqni | tgvgndenms |
| 1921 | ntwkflshst | dslnkiskvn | estesltdeg | vgtdmnegql | lgdfeieskq | leaeswsrii |
| 1981 | dskflkqqkk | dvvkrqeviy | elmqtefhhv | rtlkimsgvy | sqgmmadllf | eqqmveklfp |
| 2041 | cldelisihs | qffqrilerk | keslvdksek | nflikrigdv | lvnqfsgena | erlkktygkf |
| 2101 | cgqhnqsvny | fkdlyakdkr | fqafvkkkms | ssvvrrlgip | ecillvtqri | tkypvlfqri |
| 2161 | lqctkdneve | qedlaqslsl | vkdvigavds | kvasyekkvr | lneiytktds | ksimrmksgq |
| 2221 | mfakedlkrk | klvrdgsvfl | knaagrlkev | qavlltdilv | flqekdqkyi | fasldqkstv |
| 2281 | islkklivre | vaheekglfl | ismgmtdpem | vevhasskee | rnswiqiiqd | tintlnrded |
| 2341 | egipseneee | kkmldtrare | lkeqlhqkdq | killileeke | mifrdmaecs | tplpedcspt |
| 2401 | hsprvlfrsn | teealkggpl | mksainevei | lqglvsgnlg | gtlgptvssp | ieqdvvgpvs |
| 2461 | lprraetfgg | fdshqmnask | ggekeegddg | qdlrrtesds | glkkggnanl | vfmlkrnseq |
| 2521 | vvqsvvhlye | llsalqgvvl | qqdsyiedqk | lvlseraltr | slsrpsslie | qekqrslekq |
| 2582 | rqdlanlqkq | qaqyleekrr | rerewearer | elrerealla | qreeevqqgq | qdlekereel |
| 2641 | qqkkgtyqyd | lerlraaqkq | lereqeqlrr | eaerlsqrqt | erdlcqvshp | htklmripsf |
| 2701 | fpspeeppsp | sapsiaksgs | ldselsvspk | rnsisrthkd | kgpfhilsst | sqtnkgpegq |
| 2761 | sqapastsas | trlfgltkpk | ekkekkkknk | tsrsqpgdgp | asevsaegee | ifc |

NP_006729. A kinase anchor protein 13 isoform 1 [gi: 31563330]

| | |
|---|---|
| LOCUS | NP_006729 2817 aa linear PRI 05-OCT-2003 |
| DEFINITION | A-kinase anchor protein 13 isoform 1; A-kinase anchoring protein; |
| | guanine nucleotide exchange factor Lbc; breast cancer nuclear |
| | receptor-binding auxiliary protein; lymphoid blast crisis oncogene |
| | [Homo sapiens]. |
| ACCESSION | NP_006729 |
| VERSION | NP_006729.4 GI: 31563330 |
| DBSOURCE | REFSEQ: accession NM 006738.4 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 2817) |
| AUTHORS | Spierings, E., Brickner, A. G., Caldwell, J. A., Zegveld, S., Tatsis, N., |
| | Blokland, E., Pool, J., Pierce, R. A., Mollah, S., Shabanowitz, J., |
| | Eisenlohr, L. C., van Veelen, P., Ossendorp, F., Hunt, D. F., Goulmy, E. |
| | and Engelhard, V. H. |
| TITLE | The minor histocompatibility antigen HA-3 arises from differential |
| | proteasome-mediated cleavage of the lymphoid blast crisis (Lbc) |
| | oncoprotein |
| JOURNAL | Blood 102 (2), 621-629 (2003) |
| MEDLINE | 22718699 |
| PUBMED | 12663445 |
| REMARK | GeneRIF: The HA-3 peptide, VTEPGTAQY, is encoded by the lymphoid |
| | blast crisis oncogene, showing for the 1st time that a |
| | leukemia-associated oncogene can give rise to immunogenic T-cell |
| | epitopes that may participate in antihost & antileukemic alloimmune |
| | responses. |
| REFERENCE | 2 (residues 1 to 2817) |
| AUTHORS | Park, B., Nguyen, N. T., Dutt, P., Merdek, K. D., Bashar, M., |
| | Sterpetti, P., Tosolini, A., Testa, J. R. and Toksoz, D. |
| TITLE | Association of Lbc Rho guanine nucleotide exchange factor with |
| | alpha-catenin-related protein, alpha-catulin/CTNNAL1, supports |
| | serum response factor activation |
| JOURNAL | J. Biol. Chem. 277 (47), 45361-45370 (2002) |
| MEDLINE | 22323310 |
| PUBMED | 12270917 |
| REMARK | GeneRIF: Results show that alpha-catulin co-expression leads to |
| | increased Lbc-induced serum response factor activation and may |
| | modulate Rho pathway signaling in vivo by providing a scaffold for |
| | the Lbc Rho guanine nucleotide exchange factor. |
| REFERENCE | 3 (residues 1 to 2817) |
| AUTHORS | Diviani, D., Soderling, J. and Scott, J. D. |
| TITLE | AKAP-Lbc anchors protein kinase A and nucleates Galpha 12-selective |
| | Rho-mediated stress fiber formation |
| JOURNAL | J. Biol. Chem. 276 (47), 44247-44257 (2001) |
| MEDLINE | 21570178 |
| PUBMED | 11546812 |
| REFERENCE | 4 (residues 1 to 2817) |
| AUTHORS | Klussmann, E., Edemir, B., Pepperle, B., Tamma, G., Henn, V., |
| | Klauschenz, E., Hundsrucker, C., Maric, K. and Rosenthal, W. |
| TITLE | Ht31: the first protein kinase A anchoring protein to integrate |
| | protein kinase A and Rho signaling |
| JOURNAL | FEBS Lett. 507 (3), 264-268 (2001) |
| MEDLINE | 21553155 |
| PUBMED | 11696353 |
| REFERENCE | 5 (residues 1 to 2817) |
| AUTHORS | Sterpetti, P., Hack, A. A., Bashar, M. P., Park, B., Cheng, S. D., |
| | Knoll, J. H., Urano, T., Feig, L. A. and Toksoz, D. |
| TITLE | Activation of the Lbc Rho exchange factor proto-oncogene by |
| | truncation of an extended C terminus that regulates transformation |
| | and targeting |
| JOURNAL | Mol. Cell. Biol. 19 (2), 1334-1345 (1999) |
| MEDLINE | 99108106 |
| PUBMED | 9891067 |
| REFERENCE | 6 (residues 1 to 2817) |
| AUTHORS | Rubino, D., Driggers, P., Arbit, D., Kemp, L., Miller, B., Coso, O., |
| | Pagliai, K., Gray, K., Gutkind, S. and Segars, J. |
| TITLE | Characterization of Brx, a novel Dbl family member that modulates |
| | estrogen receptor action |
| JOURNAL | Oncogene 16 (19), 2513-2526 (1998) |
| MEDLINE | 98288806 |
| PUBMED | 9627117 |
| REFERENCE | 7 (residues 1 to 2817) |
| AUTHORS | Toksoz, D. and Williams, D. A. |
| TITLE | Novel human oncogene lbc detected by transfection with distinct |
| | homology regions to signal transduction products |
| JOURNAL | Oncogene 9 (2), 621-628 (1994) |
| MEDLINE | 94119604 |
| PUBMED | 8290273 |
| REFERENCE | 8 (residues 1 to 2817) |
| AUTHORS | Carr, D. W., Hausken, Z. E., Fraser, I. D., Stofko-Hahn, R. E. and |
| | Scott, J. D. |
| TITLE | Association of the type II cAMP-dependent protein kinase with a |
| | human thyroid RII-anchoring protein. Cloning and characterization |
| | of the RII-binding domain |
| JOURNAL | J. Biol. Chem. 267 (19), 13376-13382 (1992) |
| MEDLINE | 92317056 |
| PUBMED | 1618839 |
| REFERENCE | 9 (residues 1 to 2817) |
| AUTHORS | Carr, D. W., Stofko-Hahn, R. E., Fraser, I. D., Bishop, S. M., Acott, T. S., |
| | Brennan, R. G. and Scott, J. D. |
| TITLE | Interaction of the regulatory subunit (RII) of cAMP-dependent |
| | protein kinase with RII-anchoring proteins occurs through an |
| | amphipathic helix binding motif |
| JOURNAL | J. Biol. Chem. 266 (22), 14188-14192 (1991) |
| MEDLINE | 91317762 |
| PUBMED | 1860836 |
| COMMENT | REVIEWED REFSEQ: This record has been curated by NCBI staff. The |
| | reference sequence was derived from AF406992.1, AB055890.1, |
| | AK091210.1, AL135297.1 and BC050312.1. |
| | On Jun 10, 2003 this sequence version replaced gi: 21493026. |
| | Summary: The A-kinase anchor proteins (AKAPs) are a group of |
| | structurally diverse proteins, which have the common function of |
| | binding to the regulatory subunit of protein kinase A (PKA) and |
| | confining the holoenzyme to discrete locations within the cell. |
| | This gene encodes a member of the AKAP family. Alternative splicing |
| | of this gene results in at least 3 transcript variants encoding |
| | different isoforms containing a dbl oncogene homology (DH) domain |
| | and a pleckstrin homology (PH) domain. The DH domain is associated |
| | with guanine nucleotide exchange activation for the Rho/Rac family |
| | of small GTP binding proteins, resulting in the conversion of the |
| | inactive GTPase to the active form capable of transducing signals. |
| | The PH domain has multiple functions. Therefore, these isoforms |
| | function as scaffolding proteins to coordinate a Rho signaling |
| | pathway and, in addition, function as protein kinase A-anchoring proteins. |
| | Transcript Variant: This variant (1) encodes the longest isoform |
| | (1). |
| FEATURES | Location/Qualifiers |
| source | 1 . . . 2817 |
| | /organism=“Homo sapiens” |
| | /db_xref=“taxon:9606” |
| | /chromosome=“15” |
| | /map=“15q24-q25” |
| Protein | 1 . . . 2817 |
| | /product=“A-kinase anchor protein 13 isoform 1” |
| | /note=“A-kinase anchoring protein; guanine nucleotide |
| | exchange factor Lbc; breast cancer nuclear |
| | receptor-binding auxiliary protein; lymphoid blast crisis |
| | oncogene” |
| Region | 166 . . . 224 |
| | /region_name=“ankyrin repeats” |
| variation | 452 |
| | /allele=“T” |
| | /allele=“M” |
| | /db_xref=“dbSNP:2061821” |
| variation | 494 |
| | /allele=“W” |
| | /allele=“R” |
| | /db_xref=“dbSNP:2061822” |
| variation | 574 |
| | /allele=“R” |
| | /allele=“C” |
| | /db_xref=“dbSNP:2061824” |
| variation | 624 |
| | /allele=“V” |
| | /allele=“G” |
| | /db_xref=“dbSNP:745191” |
| variation | 689 |
| | /allele=“K” |
| | /allele=“E” |
| | /db_xref=“dbSNP:7177107” |
| variation | 845 |
| | /allele=“V” |
| | /allele=“A” |
| | /db_xref=“dbSNP:4075256” |
| variation | 897 |
| | /allele=“V” |
| | /allele=“M” |
| | /db_xref=“dbSNP:4075254” |
| variation | 1062 |
| | /allele=“P” |
| | /allele=“A” |
| | /db_xref=“dbSNP:4843074” |
| variation | 1086 |
| | /allele=“N” |
| | /allele=“D” |
| | /db_xref=“dbSNP:4843075” |
| variation | 1216 |
| | /allele=“T” |
| | /allele=“M” |
| | /db_xref=“dbSNP:7162168” |
| Region | 1792 . . . 1830 |
| | /region name=“C1 homology region” |
| Region | 2002 . . . 2193 |
| | /region_name=“Guanine nucleotide exchange factor for |
| | Rho/Rac/Cdc42-like GTPases” |
| | /note=“RhoGEF” |
| | /db_xref=“CDD:smart00325” |
| variation | 2183 |
| | /allele=“N” |
| | /allele=“S” |
| | /db_xref=“dbSNP:3743323” |
| variation | 2461 |
| | /allele=“G” |
| | /allele=“S” |
| | /db_xref=“dbSNP:2241268” |
| variation | 2805 |
| | /allele=“A” |
| | /allele=“T” |
| | /db_xref=“dbSNP:2614668” |
| CDS | 1 . . . 2817 |
| | /gene=“AKAP13” |
| | /coded_by=“NM_006738.4:171..8624” |
| | /note=“go_component: membrane fraction [goid 0005624] |
| | [evidence E]; |
| | go_function: protein kinase A anchoring activity [goid |
| | 0005079] [evidence E] [pmid 1618839]; |
| | go_function: protein binding [goid 0005515] [evidence E] |
| | [pmid 1860836]; |
| | go function: Rho guanyl-nucleotide exchange factor |
| | activity [goid 0005089] [evidence NR]; |
| | go_function: signal transducer activity [goid 0004871] |
| | [evidence P]; |
| | go_process: oncogenesis [goid 0007048] [evidence P]; |
| | go_process: signal transduction [goid 0007165] [evidence |
| | NR]” |
| | /db_xref=“GeneID:11214” |
| | /db_xref=“LocusID:11214” |
| | /db_xref=“MIM:604686” |

Origin

| | | | | | | | |
|---|---|---|---|---|---|---|---|
| 1 | mklnpqqapl | ygdcvvtvll | aeedkaeddv | vfylvflgst | lrhctstrkv | ssdtletiap | [SEQ ID NO:17] |
| 61 | ghdccetvkv | qlcaskegip | vfvvaeedfh | fvqdeaydaa | qflatsagnq | qalnftrfld | |
| 121 | qsgppsgdvn | sldkklvlaf | rhlklptewn | vlgtdqslhd | agpretlmhf | avrlgllrlt | |
| 181 | wfllqkpggr | galsihnqeg | atpvslaler | gyhklhqllt | eenagepdsw | sslsyeipyg | |
| 241 | dcsvrhhrel | diytltsesd | shhehpfpgd | gctgpifklm | niqqqlmktn | lkqmdslmpl | |
| 301 | mmtaqdpssa | petdgqflpc | apeptdpqrl | ssseetestq | ccpgspvaqt | espcdlssiv | |
| 361 | eeentdrscr | kknkgverkg | eevepapivd | sgtvsdqdsc | lqslpdcgvk | gteglsscgn | |
| 421 | rneetgtkss | gmptdqesls | sgdavlqrdl | vmepgtaqys | sggelggist | tnvstpdtag | |
| 481 | emehglmnpd | atvwknvlqg | gestkerfen | snigtagasd | vhvtskpvdk | isvpncapaa | |
| 541 | ssldgnkpae | sslafsneet | stektaetet | srsreesada | pvdqnsvvip | aaakdkisdg | |
| 601 | lepytllaag | igeamspsdl | allgleedvm | phqnsetnss | haqsqkgkss | picsttgddk | |
| 661 | lcadsacqqn | tvtssgdlva | klcdnivses | esttarqpss | qdppdashce | dpqahtvtsd | |
| 721 | pvrdtqerad | fcpfkvvdnk | gqrkdvkldk | pltnmlevvs | hphpvvpkme | kelvpdqavi | |
| 781 | sdstfslans | pgsesvtkdd | alsfvpsqke | kgtatpelht | atdyrdgpdg | nsnepdtrpl | |
| 841 | edravglsts | staaelqhgm | gntsltglgg | ehegpappai | pealnikgnt | dsslqsvgka | |
| 901 | tlaldsvlte | egkllvvses | saaqeqdkdk | avtcssiken | alssgtlqee | qrtpppgqdt | |
| 961 | qqfheksisa | dcakdkalql | snspgassaf | lkaetehnke | vapqvslltq | ggaaqslvpp | |
| 1021 | gaslatesrq | ealgaehnss | allpcllpdg | sdgsdalncs | qpspldvgvk | ntqsqgktsa | |
| 1081 | cevsgdvtvd | vtgvnalqgm | aeprrenish | ntqdilipnv | llsqeknavl | glpvalqdka | |
| 1141 | vtdpqgvgtp | emipldwekg | klegadhsct | mgdaeeaqid | deahpvllqp | vakelptdme | |
| 1201 | lsahddgapa | gvrevmrapp | sgrerstpsl | pcmvsaqdap | lpkgadliee | aasrivdavi | |
| 1261 | eqvkaagall | tegeachmsl | sspelgpltk | glesaftekv | stfppgeslp | mgstpeeatg | |
| 1321 | slagcfagre | epekiilpvq | gpepaaempd | vkaedevdfr | assiseevav | gsiaatlkmk | |
| 1381 | qgpmtqainr | enwctiepcp | daasllaskq | specenfldv | glgrectskq | gvlkresgsd | |
| 1441 | sdlfhspsdd | mdsiifpkpe | eehlacditg | sssstddtas | ldrhsshgsd | vslsqilkpn | |
| 1501 | rsrdrqsldg | fyshgmgaeg | resesepadp | gdveeeemds | itevpancsv | lrssmrslsp | |
| 1561 | frrhswgpgk | naasdaemnh | rsmswcpsgv | qysaglsadf | nyrsfslegl | tggagvgnkp | |
| 1621 | ssslevssan | aeelrhpfsg | eervdslvsl | seedlesdqr | ehrmfdqqic | hrskqqgfny | |
| 1681 | ctsaissplt | ksislmtish | pgldnsrpfh | stfhntsanl | tesiteenyn | flphspskkd | |
| 1741 | sewksgtkvs | rtfsyiknkm | ssskkskeke | kekdkikeke | kdskdkekdk | ktvnghtfss | |
| 1801 | ipvvgpiscs | qcmkpftnkd | aytcancsaf | vhkgcresla | scakvkmkqp | kgslqahdts | |
| 1861 | slptvimrnk | psqpkerprs | avllvdetat | tpifanrrsq | qsvslsksvs | iqnitgvgnd | |
| 1921 | enmsntwkfl | shstdslnki | skvnestesl | tdegvgtdmn | egqllgdfei | eskqleaesw | |
| 1981 | sriidskflk | qqkkdvvkrq | eviyelmqte | fhhvrtlkim | sgvysqgmma | dllfeqqmve | |
| 2041 | klfpcldeli | sihsqffqri | lerkkeslvd | kseknflikr | igdvlvnqfs | genaerlkkt | |
| 2101 | ygkfcgqhnq | svnyfkdlya | kdkrfqafvk | kkmsssvvrr | lgipecillv | tqritkypvl | |
| 2161 | fqrilqctkd | neveqedlaq | slslvkdvig | avdskvasye | kkvrlneiyt | ktdsksimrm | |
| 2221 | ksgqmfaked | lkrkklvrdg | svflknaagr | lkevqavllt | dilvflqekd | qkyifasldq | |
| 2281 | kstvislkkl | ivrevaheek | glflismgmt | dpemvevhas | skeernswiq | iiqdtintln | |
| 2341 | rdedegipse | neeekkmldt | rarelkeqlh | qkdqkillll | eekemifrdm | aecstplped | |
| 2401 | cspthsprvl | frsnteealk | ggplmksain | eveilqglvs | gnlggtlgpt | vsspieqdvv | |
| 2461 | gpvslprrae | tfggfdshqm | naskggekee | gddgqdlrrt | esdsglkkgg | nanlvfmlkr | |
| 2521 | nseqvvqsvv | hlyellsalq | gvvlqqdsyi | edqklvlser | altrslsrps | slieqekqrs | |
| 2581 | lekqrqdlan | lqkqqaqyle | ekrrrerewe | arerelrere | allaqreeev | qqgqqdleke | |
| 2641 | reelqqkkgt | yqydlerlra | aqkqlereqe | qlrreaerls | qrqterdlcq | vshphtklmr | |
| 2701 | ipsffpspee | ppspsapsia | ksgsldsels | vspkrnsisr | thkdkgpfhi | lsstsqtnkg | |
| 2761 | pegqsqapas | tsastrlfgl | tkpkekkekk | kknktsrsqp | gdgpasevsa | egeeifc | |

NP_658913. A-kinase anchor protein 13 isoform 3 [gi: 21493031]

| | |
|---|---|
| LOCUS | NP_658913 1058 aa linear PRI 05-OCT-2003 |
| DEFINITION | A-kinase anchor protein 13 isoform 3; A-kinase anchoring protein; |
| | guanine nucleotide exchange factor Lbc; breast cancer nuclear |
| | receptor-binding auxiliary protein; lymphoid blast crisis oncogene |
| | [Homo sapiens]. |
| ACCESSION | NP_658913 |
| VERSION | NP_658913.1 GI: 21493031 |
| DBSOURCE | REFSEQ: accession NM 144767.3 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 1058) |
| AUTHORS | Spierings, E., Brickner, A. G., Caldwell, J. A., Zegveld, S., Tatsis, N., |
| | Blokland, E., Pool, J., Pierce, R. A., Mollah. S., Shabanowitz, J., |
| | Eisenlohr, L. C., van Veelen, P., Ossendorp, F., Hunt, D. F., Goulmy, E. |
| | and Engelhard, V. H. |
| TITLE | The minor histocompatibility antigen HA-3 arises from differential |
| | proteasome-mediated cleavage of the lymphoid blast crisis (Lbc) |
| | oncoprotein |
| JOURNAL | Blood 102 (2), 621-629 (2003) |
| MEDLINE | 22718699 |
| PUBMED | 12663445 |
| REMARK | GeneRIF: The HA-3 peptide, VTEPGTAQY, is encoded by the lymphoid |
| | blast crisis oncogene, showing for the 1st time that a |
| | leukemia-associated oncogene can give rise to immunogenic T-cell |
| | epitopes that may participate in antihost & antileukemic alloimmune |
| | responses. |
| REFERENCE | 2 (residues 1 to 1058) |
| AUTHORS | Park, B., Nguyen, N. T., Dutt, P., Merdek, K. D., Bashar, M., |
| | Sterpetti, P., Tosolini, A., Testa, J. R. and Toksoz, D. |
| TITLE | Association of Lbc Rho guanine nucleotide exchange factor with |
| | alpha-catenin-related protein, alpha-catulin/CTNNAL1, supports |
| | serum response factor activation |
| JOURNAL | J. Biol. Chem. 277 (47), 45361-45370 (2002) |
| MEDLINE | 22323310 |
| PUBMED | 12270917 |
| REMARK | GeneRIF: Results show that alpha-catulin co-expression leads to |
| | increased Lbc-induced serum response factor activation and may |
| | modulate Rho pathway signaling in vivo by providing a scaffold for |
| | the Lbc Rho guanine nucleotide exchange factor. |
| REFERENCE | 3 (residues 1 to 1058) |
| AUTHORS | Diviani, D., Soderling, J. and Scott, J. D. |
| TITLE | AKAP-Lbc anchors protein kinase A and nucleates Galpha 12-selective |
| | Rho-mediated stress fiber formation |
| JOURNAL | J. Biol. Chem. 276 (47), 44247-44257 (2001) |
| MEDLINE | 21570178 |
| PUBMED | 11546812 |
| REFERENCE | 4 (residues 1 to 1058) |
| AUTHORS | Klussmann, E., Edemir, B., Pepperle, B., Tamma, G., Henn, V., |
| | Klauschenz, E., Hundsrucker, C., Maric, K. and Rosenthal, W. |
| TITLE | Ht31: the first protein kinase A anchoring protein to integrate |
| | protein kinase A and Rho signaling |
| JOURNAL | FEBS Lett. 507 (3), 264-268 (2001) |
| MEDLINE | 21553155 |
| PUBMED | 11696353 |
| REFERENCE | 5 (residues 1 to 1058) |
| AUTHORS | Sterpetti, P., Hack, A. A., Bashar, M. P., Park, B., Cheng, S. D., |
| | Knoll, J. H., Urano, T., Feig, L. A. and Toksoz, D. |
| TITLE | Activation of the Lbc Rho exchange factor proto-oncogene by |
| | truncation of an extended C terminus that regulates transformation |
| | and targeting |
| JOURNAL | Mol. Cell. Biol. 19 (2), 1334-1345 (1999) |
| MEDLINE | 99108106 |
| PUBMED | 9891067 |
| REFERENCE | 6 (residues 1 to 1058) |
| AUTHORS | Rubino, D., Driggers, P., Arbit, D., Kemp, L., Miller, B., Coso, O., |
| | Pagliai, K., Gray, K., Gutkind, S. and Segars, J. |
| TITLE | Characterization of Brx, a novel Dbl family member that modulates |
| | estrogen receptor action |
| JOURNAL | Oncogene 16 (19), 2513-2526 (1998) |
| MEDLINE | 98288806 |
| PUBMED | 9627117 |
| REFERENCE | 7 (residues 1 to 1058) |
| AUTHORS | Toksoz, D. and Williams, D. A. |
| TITLE | Novel human oncogene lbc detected by transfection with distinct |
| | homology regions to signal transduction products |
| JOURNAL | Oncogene 9 (2), 621-628 (1994) |
| MEDLINE | 94119604 |
| PUBMED | 8290273 |
| REFERENCE | 8 (residues 1 to 1058) |
| AUTHORS | Carr, D. W., Hausken, Z. E., Fraser, I. D., Stofko-Hahn, R. E. and |
| | Scott, J. D. |
| TITLE | Association of the type II cAMP-dependent protein kinase with a |
| | human thyroid RII-anchoring protein. Cloning and characterization |
| | of the RII-binding domain |
| JOURNAL | J. Biol. Chem. 267 (19), 13376-13382 (1992) |
| MEDLINE | 92317056 |
| PUBMED | 1618839 |
| REFERENCE | 9 (residues 1 to 1058) |
| AUTHORS | Carr, D. W., Stofko-Hahn, R. E., Fraser, I. D., Bishop, S. M., Acott, T. S., |
| | Brennan, R. G. and Scott, J. D. |
| TITLE | Interaction of the regulatory subunit (RII) of cAMP-dependent |
| | protein kinase with RII-anchoring proteins occurs through an |
| | amphipathic helix binding motif |
| JOURNAL | J. Biol. Chem. 266 (22), 14188-14192 (1991) |
| MEDLINE | 91317762 |
| PUBMED | 1860836 |
| COMMENT | REVIEWED REFSEQ: This record has been curated by NCBI staff. The |
| | reference sequence was derived from AF127481.1, AB055890.1, |
| | AK091210.1 and AL135297.1. |
| | Summary: The A-kinase anchor proteins (AKAPs) are a group of |
| | structurally diverse proteins, which have the common function of |
| | binding to the regulatory subunit of protein kinase A (PKA) and |
| | confining the holoenzyme to discrete locations within the cell. |
| | This gene encodes a member of the AKAP family. Alternative splicing |
| | of this gene results in at least 3 transcript variants encoding |
| | different isoforms containing a dbl oncogene homology (DH) domain |
| | and a pleckstrin homology (PH) domain. The DH domain is associated |
| | with guanine nucleotide exchange activation for the Rho/Rac family |
| | of small GTP binding proteins, resulting in the conversion of the |
| | inactive GTPase to the active form capable of transducing signals. |
| | The PH domain has multiple functions. Therefore, these isoforms |
| | function as scaffolding proteins to coordinate a Rho signaling |
| | pathway and, in addition, function as protein kinase A-anchoring |
| | proteins. |
| | Transcript Variant: This variant (3) lacks most of the 5′ exons and |
| | has an alternate 5′ exon, as compared to variant 1. It uses a |
| | downstream in-frame start codon, and the resulting isoform 3 is |
| | shorter but has an identical C-terminus, compared to isoform 1. |
| | Sequence Note: The sequence AF127481.1 is a chimeric mRNA clone. |
| | Only the AKAP13 region was propagated into this RefSeq record. |
| FEATURES | Location/Qualifiers |
| source | 1 . . . 1058 |
| | /organism=“Homo sapiens” |
| | /db_xref=“taxon:9606” |
| | /chromosome=“15” |
| | /map=“15q24-q25” |
| Protein | 1 . . . 1058 |
| | /product=“A-kinase anchor protein 13 isoform 3” |
| | /note=“A-kinase anchoring protein; guanine nucleotide |
| | exchange factor Lbc; breast cancer nuclear |
| | receptor-binding auxiliary protein; lymphoid blast crisis |
| | oncogene” |
| Region | 33 . . . 71 |
| | /region_name=“C1 homology region” |
| Region | 243 . . . 434 |
| | /region name=“Guanine nucleotide exchange factor for |
| | Rho/Rac/Cdc42-like GTPases” |
| | /note=“RhoGEF” |
| | /db_xref=“CDD:smart00325” |
| variation | 424 |
| | /allele=“N” |
| | /allele=“S” |
| | /db_xref=“dbSNP:3743323” |
| variation | 702 |
| | /allele=“G” |
| | /allele=“S” |
| | /db_xref=“dbSNP:2241268” |
| variation | 1046 |
| | /allele=“A” |
| | /allele=“T” |
| | /db_xref=“dbSNP:2614668” |
| CDS | 1 . . . 1058 |
| | /gene=“AKAP13” |
| | /coded_by=“NM_144767.3:227..3403” |
| | /note=“go component: membrane fraction [goid 0005624] |
| | [evidence E]; |
| | go function: protein kinase A anchoring activity [goid |
| | 0005079] [evidence E] [pmid 1618839]; |
| | go function: protein binding [goid 0005515] [evidence E] |
| | [pmid 1860836]; |
| | go function: Rho guanyl-nucleotide exchange factor |
| | activity [goid 0005089] [evidence NR]; |
| | go function: signal transducer activity [goid 0004871] |
| | [evidence P]; |
| | go_process: oncogenesis [goid 0007048] [evidence P]; |
| | go_process: signal transduction [goid 0007165] [evidence |
| | NR]” |
| | /db_xref=“GeneID:11214” |
| | /db_xref=“LocusID:11214” |
| | /db_xref=“MIM:604686” |

Origin

| | | | | | | | |
|---|---|---|---|---|---|---|---|
| 1 | mssskkskek | ekekdkikek | ekdskdkekd | kktvnghtfs | sipvvgpisc | sqcmkpftnk | [SEQ ID NO:18] |
| 61 | daytcancsa | fvhkgcresl | ascakvkmkq | pkgslqahdt | sslptvimrn | kpsqpkerpr | |
| 121 | savllvdeta | ttpifanrrs | qqsvslsksv | siqnitgvgn | denmsntwkf | lshstdslnk | |
| 181 | iskvnestes | ltdegvgtdm | negqllgdfe | ieskqleaes | wsriidskfl | kqqkkdvvkr | |
| 241 | qeviyelmqt | efhhvrtlki | msgvysqgmm | adllfeqqmv | eklfpcldel | isihsqffqr | |
| 301 | ilerkkeslv | dkseknflik | rigdvlvnqf | sgenaerlkk | tygkfcgqhn | qsvnyfkdly | |
| 361 | akdkrfqafv | kkkmsssvvr | rlgipecill | vtqritkypv | lfqrilqctk | dneveqedla | |
| 421 | qslslvkdvi | gavdskvasy | ekkvrlneiy | tktdsksimr | mksgqmfake | dlkrkklvrd | |
| 481 | gsvflknaag | rlkevqavll | tdilvflqek | dqkyifasld | qkstvislkk | livrevahee | |
| 541 | kglflismgm | tdpemvevha | sskeernswi | qiiqdtintl | nrdedegips | eneeekkmld | |
| 601 | trarelkeql | hqkdqkilll | leekemifrd | maecstplpe | dcspthsprv | lfrsnteeal | |
| 661 | kggplmksai | neveilqglv | sgnlggtlgp | tvsspieqdv | vgpvslprra | etfggfdshq | |
| 721 | mnaskggeke | egddgqdlrr | tesdsglkkg | gnanlvfmlk | rnseqvvqsv | vhlyellsal | |
| 781 | qgvvlqqdsy | iedqklvlse | raltrslsrp | sslieqekqr | slekqrqdla | nlqkqqaqyl | |
| 841 | eekrrrerew | earerelrer | eallaqreee | vqqgqqdlek | ereelqqkkg | tyqydlerlr | |
| 901 | aaqkqlereq | eqlrreaerl | sqrqterdlc | qvshphtklm | ripsffpspe | eppspsapsi | |
| 961 | aksgsldsel | svspkrnsis | rthkdkgpfh | ilsstsqtnk | gpegqsqapa | stsastrlfg | |
| 1021 | ltkpkekkek | kkknktsrsq | pgdgpasevs | aegeeifc | | | |

IJ. ATP Citrate Lyase

ATP citrate lyase has been identified as an Akt substrate that interacts with Akt. ATP citrate lyase is associated with reference sequence NP_{—}001087 (GenBank Accession No. 4501865). Other relevant sequences include rat citrate lyase associated with reference sequence NP_{—}058683 (GenBank Accession No. 8392839).

NP_001087. ATP citrate lyase [gi: 4501865]

| | |
|---|---|
| LOCUS | NP_001087 1105 aa linear PRI 04-OCT-2003 |
| DEFINITION | ATP citrate lyase [Homo sapiens]. |
| ACCESSION | NP_001087 |
| VERSION | NP_001087.1 GI: 4501865 |
| DBSOURCE | REFSEQ: accession NM 001096.1 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 1105) |
| AUTHORS | Couch, F. J., Abel, K. J., Brody, L. C., Boehnke, M., Collins, F. S. and |
| | Weber, B. L. |
| TITLE | Localization of the gene for ATP citrate lyase (ACLY) distal to |
| | gastrin(GAS) and proximal to D17S856 on chromosome 17q12-q21 |
| JOURNAL | Genomics 21 (2), 444-446 (1994) |
| MEDLINE | 94375075 |
| PUBMED | 8088842 |
| REFERENCE | 2 (residues 1 to 1105) |
| AUTHORS | Elshourbagy, N. A., Near, J. C., Kmetz, P. J., Wells, T. N., Groot, P. H., |
| | Saxty, B. A., Hughes, S. A., Franklin, M. and Gloger, I. S. |
| TITLE | Cloning and expression of a human ATP-citrate lyase cDNA |
| JOURNAL | Eur. J. Biochem. 204 (2), 491-499 (1992) |
| MEDLINE | 92174902 |
| PUBMED | 1371749 |
| COMMENT | REVIEWED REFSEQ: This record has been curated by NCBI staff. The |
| | reference sequence was derived from X64330.1. |
| | Summary: ATP citrate lyase is the primary enzyme responsible for |
| | the synthesis of cytosolic acetyl-CoA in many tissues. The enzyme |
| | is a tetramer (relative molecular weight approximately 440,000) of |
| | apparently identical subunits. It catalyzes the formation of |
| | acetyl-CoA and oxaloacetate from citrate and CoA with a concomitant |
| | hydrolysis of ATP to ADP and phosphate. The product, acetyl-CoA, |
| | serves several important biosynthetic pathways, including |
| | lipogenesis and cholesterogenesis. In nervous tissue, ATP |
| | citrate-lyase may be involved in the biosynthesis of acetylcholine. |
| FEATURES | Location/Qualifiers |
| source | 1. . . 1105 |
| | /organism=“Homo sapiens” |
| | /db_xref=“taxon:9606” |
| | /chromosome=“17” |
| | /map=“17q12-q21” |
| Protein | 1 . . . 1105 |
| | /product=“ATP citrate lyase” |
| | /EC_number=“4.1.3.8” |
| Region | 6 . . . 415 |
| | /region name=“Succinyl-CoA synthetase, beta subunit |
| | [Energy production and conversion]” |
| | /note=“SucC” |
| | /db_xref=“CDD:COG0045” |
| Region | 554 . . . 809 |
| | /region_name=“Succinyl-CoA synthetase, alpha subunit |
| | [Energy production and conversion]” |
| | /note=“SucD” |
| | /db_xref=“CDD:COG0074” |
| CDS | 1 . . . 1105 |
| | /gene=“ACLY” |
| | /coded_by=“NM_001096.1:85..3402” |
| | /note=“go_component: citrate lyase complex [goid 0009346] |
| | [evidence TAS] [pmid 1371749]; |
| | go_function: ATP citrate synthase activity [goid 0003878] |
| | [evidence TAS] [pmid 1371749]; |
| | go function: transferase activity [goid 0016740] [evidence |
| | IEA]; |
| | go_function: ATP citrate synthase activity [goid 0046913] |
| | [evidence IEA]; |
| | go_function: ATP binding [goid 0005524] [evidence IEA]; |
| | go_function: magnesium ion binding [goid 0000287] |
| | [evidence IEA]; |
| | go_function: lyase activity [goid 0016829] [evidence IEA]; |
| | go_function: citrate (Si)-synthase activity [goid 0004108] |
| | [evidence IEA]; |
| | go_process: ATP catabolism [goid 0006200] [evidence TAS] |
| | [pmid 1371749]; |
| | go_process: coenzyme A metabolism [goid 0015936] [evidence |
| | TAS] [pmid 1371749]; |
| | go_process: citrate metabolism [goid 0006101] [evidence |
| | TAS] [pmid 1371749]; |
| | go_process: metabolism [goid 0008152] [evidence IEA]; |
| | go_process: lipid biosynthesis [goid 0008610] [evidence |
| | IEA]; |
| | go_process: tricarboxylic acid cycle [goid 0006099] |
| | [evidence IEA]” |
| | /db_xref=“GeneID:47” |
| | /db_xref=“LocusID:47” |
| | /db_xref=“MIM:108728” |

Origin

| | | | | | | | |
|---|---|---|---|---|---|---|---|
| 1 | msakaiseqt | gkellykfic | ttsaiqnrfk | yarvtpdtdw | arllqdhpwl | lsqnlvvkpd | [SEQ ID NO:19] |
| 61 | qlikrrgklg | lvgvdltldg | vkswlkprlg | qeatvgkatg | flknfliepf | aphsqaeefy | |
| 121 | vciyatregd | yvlfhheggv | dvgdvdakaq | kllvgvdekl | npedikkhll | vhapddkkei | |
| 181 | lasfisglfn | fyedlyftyl | einplvvtkd | gvyvldlaak | vdatadyick | vkwgdiefpp | |
| 241 | pfgrvaypee | ayiadldaks | gaslkltlln | pkgriwtmva | gggasvvysd | ticdlggvne | |
| 301 | lanygeysga | pseqqtydya | ktilslmtre | khpdgkilii | ggsianftnv | aatfkgivra | |
| 361 | irdyqgplke | hevtifvrrg | gpnyqeglrv | mgevgkttgi | pihvfgteth | mtaivgmawa | |
| 421 | paipnqppta | ahtanfllna | qretstpaps | rtasfyesmv | devradevap | akkakpampq | |
| 481 | dsvpsprslq | gksttlfsrh | tkaivwgmqt | ravqgmldfd | yvcsrdepsv | aamvypftgd | |
| 541 | hkqkfywghk | eilipvfknm | adamrkhpev | dvlinfaslr | saydstmetm | nyaqirtiai | |
| 601 | iaegipealt | rklikkadqk | gvtiigpatv | ggikpgcfki | gntggmldni | lasklypqaa | |
| 661 | vayvsrsggm | snelnniisr | ttdgvyegva | iggdrypgst | fmdhvlryqd | tpgvkmivvl | |
| 721 | geiggteeyk | isrgikegrl | tkpivcwcig | tcatmfssev | qfghagacan | qasetavakn | |
| 781 | qalkeagvfv | prsfdelgei | iqsvyedlva | ngvivpaqev | ppptvpmdys | warelglirk | |
| 841 | pasfmtsicd | ergqeliyag | mpitevfkee | mgiggalgll | wfqkrlpkys | cqfiemclmv | |
| 901 | tadhgpavsg | ahntiicart | avelvsslts | glltigdrfg | galdaaakmf | skafdsgiip | |
| 961 | mefvnkmkke | gklimgighr | vksinnpdmr | vqilkdyvrq | hfpatplldy | alevekitts | |
| 1021 | kkpnlilnvd | gligvafvdm | lrncgsftre | eadeyidiga | lngifvlgrs | mgfighyldq | |
| 1081 | krlkqglyrh | pwddisyvlp | ehmsm | | | | |

IK Chromodomain Helicase DNA Binding Protein 4 (Mi-2b)

Chromodomain helicase DNA binding protein 4 (Mi-2b) has been identified as an Akt substrate that interacts with Akt. Mi-2b is associated with reference sequence NP_{—}001264 (GenBank Accession No. 4557453).

NP_001264. chromodomain helicase DNA binding protein 4; Mi-2b [gi: 4557453]

| | |
|---|---|
| LOCUS | NP_001264 1912 aa linear PRI 04-OCT-2003 |
| DEFINITION | chromodomain helicase DNA binding protein 4; Mi-2b [Homo sapiens]. |
| ACCESSION | NP_001264 |
| VERSION | NP_001264.1 GI: 4557453 |
| DBSOURCE | REFSEQ: accession NM 001273.1 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 1912) |
| AUTHORS | Zhang, Y., LeRoy, G., Seelig, H. P., Lane, W. S. and Reinberg, D. |
| TITLE | The dermatomyositis-specific autoantigen Mi2 is a component of a |
| | complex containing histone deacetylase and nucleosome remodeling |
| | activities |
| JOURNAL | Cell 95 (2), 279-289 (1998) |
| MEDLINE | 99005195 |
| PUBMED | 9790534 |
| REFERENCE | 2 (residues 1 to 1912) |
| AUTHORS | Woodage, T., Basrai, M. A., Baxevanis, A. D., Hieter, P. and Collins, F. S. |
| TITLE | Characterization of the CHD family of proteins |
| JOURNAL | Proc. Natl. Acad. Sci. U.S.A. 94 (21), 11472-11477 (1997) |
| MEDLINE | 97470991 |
| PUBMED | 9326634 |
| REFERENCE | 3 (residues 1 to 1912) |
| AUTHORS | Seelig, H. P., Renz, M., Targoff, I. N., Ge, Q. and Frank, M. B. |
| TITLE | Two forms of the major antigenic protein of the |
| | dermatomyositis-specific Mi-2 autoantigen |
| JOURNAL | Arthritis Rheum. 39 (10), 1769-1771 (1996) |
| MEDLINE | 97000821 |
| PUBMED | 8843877 |
| REFERENCE | 4 (residues 1 to 1912) |
| AUTHORS | Seelig, H. P., Moosbrugger, I., Ehrfeld, H., Fink, T., Renz, M. and |
| | Genth, E. |
| TITLE | The major dermatomyositis-specific Mi-2 autoantigen is a presumed |
| | helicase involved in transcriptional activation |
| JOURNAL | Arthritis Rheum. 38 (10), 1389-1399 (1995) |
| MEDLINE | 96017437 |
| PUBMED | 7575689 |
| REFERENCE | 5 (residues 1 to 1912) |
| AUTHORS | Ge, Q., Nilasena, D. S., O'Brien, C. A., Frank, M. B. and Targoff, I. N. |
| TITLE | Molecular analysis of a major antigenic region of the 240-kD |
| | protein of Mi-2 autoantigen |
| JOURNAL | J. Clin. Invest. 96 (4), 1730-1737 (1995) |
| MEDLINE | 96013633 |
| PUBMED | 7560064 |
| COMMENT | REVIEWED REFSEQ: This record has been curated by NCBI staff. The |
| | reference sequence was derived from X86691.1. |
| | Summary: The CHD family of proteins is characterized by the |
| | presence of chromo (chromatin organization modifier) domains and |
| | SNF2-related helicase/ATPase domains. Patients with dermatomyositis |
| | develop antibodies against the nuclear antigen chromodomain |
| | helicase DNA binding protein 4. The protein exists in a complex |
| | containing histone deacetylase and nucleosome remodeling activities, |
| | suggesting a role for chromatin reorganization in cancer metastasis. |
| FEATURES | Location/Qualifiers |
| source | 1 . . . 1912 |
| | /organism=“Homo sapiens” |
| | /db_xref=“taxon:9606” |
| | /chromosome=“12” |
| | /map=“12p13” |
| Protein | 1 . . . 1912 |
| | /product=“chromodomain helicase DNA binding protein 4” |
| | /note=“Mi-2b” |
| variation | 139 |
| | /allele=“D” |
| | /allele=“E” |
| | /db_xref=“dbSNP:1639122” |
| Region | 372 . . . 417 |
| | /region_name=“PHD-finger. PHD folds into an interleaved |
| | type of Zn-finger chelating 2 Zn ions in a similar manner |
| | to that of the RING and FYVE domains” |
| | /note=“PHD” |
| | /db_xref=“CDD:pfam00628” |
| Region | 451 . . . 496 |
| | /region_name=“PHD-finger. PHD folds into an interleaved |
| | type of Zn-finger chelating 2 Zn ions in a similar manner |
| | to that of the RING and FYVE domains” |
| | /note=“PHD” |
| | /db_xref=“CDD:pfam00628” |
| Region | 540 . . . 579 |
| | /region name=“Chromatin organization modifier domain” |
| | /note=“CHROMO” |
| | /db_xref=“CDD:smart00298” |
| Region | 622 . . . 676 |
| | /region name=“Chromatin organization modifier domain” |
| | /note=“CHROMO” |
| | /db_xref=“CDD:smart00298” |
| Region | 708 . . . 1222 |
| | /region_name=“Superfamily II DNA/RNA helicases, SNF2 |
| | family [Transcription/DNA replication, recombination, |
| | and repair]” |
| | /note=“HepA” |
| | /db_xref=“CDD:COG0553” |
| CDS | 1 . . . 1912 |
| | /gene=“CHD4” |
| | /coded_by=“NM_001273.1:90..5828” |
| | /note=“go component: chromatin [goid 0005717] [evidence |
| | IEA]; |
| | go_component: nucleus [goid 0005634] [evidence IEA]; |
| | go_function: ATP dependent DNA helicase activity [goid |
| | 0004003] [evidence TAS] [pmid 9326634]; |
| | go function: zinc ion binding [goid 0008270] [evidence |
| | TAS] [pmid 7560064]; |
| | go function: DNA binding [goid 0003677] [evidence P] [pmid |
| | 9326634]; |
| | go function: chromatin binding [goid 0003682] [evidence |
| | IEA]; |
| | go_function: ATP binding [goid 0005524] [evidence IEA]; |
| | go_process: chromosome organization and biogenesis (sensu |
| | Eukarya) [goid 0007001] [evidence TAS] [pmid 9326634]; |
| | go_process: regulation of transcription from Pol II |
| | promoter [goid 0006357] [evidence TAS] [pmid 9326634]; |
| | go_process: chromatin assembly/disassembly [goid 0006333] |
| | [evidence IEA]; |
| | go_process: chromatin modification [goid 0016568] |
| | [evidence IEA]” |
| | /db_xref=“GeneID:1108” |
| | /db_xref=“LocusID:1108” |
| | /db_xref=“MIM:603277” |

Origin

| | | | | | | | |
|---|---|---|---|---|---|---|---|
| 1 | masglgspsp | csagseeedm | dallnnslpp | phpeneedpe | edlsetetpk | lkkkkkpkkp | [SEQ ID NO:20] |
| 61 | rdpkipkskr | qkkermllcr | qlgdssgegp | efveeeeeva | lrsdsegsdy | tpgkkkkkkl | |
| 121 | gpkkekksks | krkeeeeedd | ddddskepks | saqlledwgm | edidhvfsee | dyrtltnyka | |
| 181 | fsqfvrplia | aknpkiavsk | mmmvlgakwr | efstnnpfkg | ssgasvaaaa | aaavavvesm | |
| 241 | vtatevappp | ppvevpirka | ktkegkgpna | rrkpkgsprv | pdakkpkpkk | vaplkiklgg | |
| 301 | fgskrkrsss | edddldvesd | fddasinsys | vsdgstsrss | rsrkklrttk | kkkkgeeevt | |
| 361 | avdgyetdhq | dycevcqqgg | eiilcdtcpr | ayhmvcldpd | mekapegkws | cphcekegiq | |
| 421 | weakednseg | eeileevggd | leeeddhhme | fcrvckdgge | llccdtcpss | yhihclnppl | |
| 481 | peipngewlc | prctcpalkg | kvqkiliwkw | gqppsptpvp | rppdadpntp | spkplegrpe | |
| 541 | rqffvkwqgm | sywhcswvse | lqlelhcqvm | frnyqrkndm | deppsgdfgg | deeksrkrkn | |
| 601 | kdpkfaemee | rfyrygikpe | wmmihrilnh | svdkkghvhy | likwrdlpyd | qaswesedve | |
| 661 | iqdydlfkqs | ywnhrelmrg | eegrpgkklk | kvklrklerp | petptvdptv | kyerqpeyld | |
| 721 | atggtlhpyq | meglnwlrfs | waqgtdtila | demglgktvq | tavflyslyk | eghskgpflv | |
| 781 | saplstiinw | erefemwapd | myvvtyvgdk | dsraiirene | fsfednairg | gkkasrmkke | |
| 841 | asvkfhvllt | syelitidma | ilgsidwacl | ivdeahrlkn | nqskffrvln | gyslqhklll | |
| 901 | tgtplqnnle | elfhllnflt | perfhnlegf | leefadiake | dqikklhdml | gphmlrrlka | |
| 961 | dvfknmpskt | elivrvelsp | mqkkyykyil | trnfealnar | gggnqvslln | vvmdlkkccn | |
| 1021 | hpylfpvaam | eapkmpngmy | dgsalirasg | kllllqkmlk | nlkegghrvl | ifsqmtkmld | |
| 1081 | lledfleheg | ykyeridggi | tgnmrqeaid | rfnapgaqqf | cfllstragg | lginlatadt | |
| 1141 | viiydsdwnp | hndiqafsra | hrigqnkkvm | iyrfvtrasv | eeritqvakk | kmmlthlvvr | |
| 1201 | pglgsktgsm | skqelddilk | fgteelfkde | atdgggdnke | gedssvihyd | dkaierlldr | |
| 1261 | nqdetedtel | qgmneylssf | kvaqyvvree | emgeeeever | eiikqeesvd | pdywekllrh | |
| 1321 | hyeqqqedla | rnlgkgkrir | kqvnyndgsq | edrdwqddqs | dnqsdysvas | eegdedfder | |
| 1381 | seaprrpsrk | glrndkdkpl | ppllarvggn | ievlgfnarq | rkaflnaimr | ygmppqdaft | |
| 1441 | tqwlvrdlrg | ksekefkayv | slfmrhlcep | gadgaetfad | gvpreglsrq | hvltrigvms | |
| 1501 | lirkkvqefe | hvngrwsmpe | laeveenkkm | sqpgspspkt | ptpstpgdtq | pntpapvppa | |
| 1561 | edgikieens | lkeeesiege | kevkstapet | aiectqapap | asedekvvve | ppegeekvek | |
| 1621 | aevkerteep | metepkgaad | vekveeksai | dltpivvedk | eekkeeeekk | evmlqngetp | |
| 1681 | kdlndekqkk | nikqrfmfni | adggftelhs | lwqneeraat | vtkktyeiwh | rrhdywllag | |
| 1741 | iinhgyarwq | diqndpryai | lnepfkgemn | rgnfleiknk | flarrfklle | qalvieeqlr | |
| 1801 | raaylnmsed | pshpsmalnt | rfaeveclae | shqhlskesm | agnkpanavl | hkvlkqleel | |
| 1861 | lsdmkadvtr | lpatiaripp | vavrlqmser | nilsrlanra | peptpqqvaq | qq | |

IL. Peripheral Benzodiazepine Receptor-Associated Protein 1

Peripheral benzodiazepine receptor-associated protein 1 has been identified as an Akt substrate that interacts with Akt. This protein is associated with reference sequence NP_{—}004749 (GenBank Accession No. 4758956). A specific sequence identified as an Akt substrate includes a hypothetical protein, similar to peripheral benzodiazepine receptor associated protein 1 and RIM-binding protein 1 (KIAA0612) (GenBank Accession No. 7513043).

HP 004749. peripheral benzodiazepine receptor-associated
protein 1 [gi: 4758956]

| | |
|---|---|
| LOCUS | NP_004749 1857 aa linear PRI 06-OCT-2003 |
| DEFINITION | peripheral benzodiazepine receptor-associated protein 1 [Homo |
| | sapiens]. |
| ACCESSION | NP_004749 |
| VERSION | NP_004749.1 GI: 4758956 |
| DBSOURCE | REFSEQ: accession NM 004758.1 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 1857) |
| AUTHORS | Galiegue, S., Jbilo, O., Combes, T., Bribes, E., Carayon, P., Le Fur, G. |
| | and Casellas, P. |
| TITLE | Cloning and characterization of PRAX-1. A new protein that |
| | specifically interacts with the peripheral benzodiazepine receptor |
| JOURNAL | J. Biol. Chem. 274 (5), 2938-2952 (1999) |
| MEDLINE | 99115641 |
| PUBMED | 9915832 |
| COMMENT | PROVISIONAL REFSEQ: This record has not yet been subject to final |
| NCBI | review. The reference sequence was derived from AF039571.1. |
| FEATURES | Location/Qualifiers |
| source | 1 . . . 1857 |
| | /organism=“Homo sapiens” |
| | /db_xref=“taxon:9606” |
| | /chromosome=“17” |
| | /map=“17q22-q23” |
| Protein | 1 . . . 1857 |
| | /product=“peripheral benzodiazepine receptor-associated |
| | protein 1” |
| Region | 125 . . . 464 |
| | /region_name=“Chromosome segregation ATPases [Cell |
| | division and chromosome partitioning]” |
| | /note=“Smc” |
| | /db_xref=“CDD:COG1196” |
| variation | 514 |
| | /allele=“Q” |
| | /allele=“R” |
| | /db_xref=“dbSNP:2072145” |
| variation | 586 |
| | /allele=“A” |
| | /allele=“T” |
| | /db_xref=“dbSNP:2072147” |
| Region | 660 . . . 718 |
| | /region name=“Src homology 3 domains” |
| | /note=“SH3” |
| | /db_xref=“CDD:smart00326” |
| Region | 885 . . . 947 |
| | /region name=“Fibronectin type 3 domain” |
| | /note=“FN3” |
| | /db_xref=“CDD:smart00060” |
| variation | 1118 |
| | /allele=“H” |
| | /allele=“L” |
| | /db_xref=“dbSNP:3744099” |
| variation | 1140 |
| | /allele=“P” |
| | /allele=“A” |
| | /db_xref=“dbSNP:2680704” |
| variation | 1253 |
| | /allele=“R” |
| | /allele=“C” |
| | /db_xref=“dbSNP:3744101” |
| Region | 1626 . . . 1691 |
| | /region name=“Src homology 3 domains” |
| | /note=“SH3” |
| | /db_xref=“CDD:smart00326” |
| Region | 1766 . . . 1827 |
| | /region name=“Src homology 3 domains” |
| | /note=“SH3” |
| | /db_xref=“CDD:smart00326” |
| variation | 1830 |
| | /allele=“G” |
| | /allele=“E” |
| | /db_xref=“dbSNP:2301868” |
| CDS | 1 . . . 1857 |
| | /gene=“BZRAP1” |
| | /coded_by=“NM_004758.1:198..5771” |
| | /note=“go_component: mitochondrion [goid 0005739] |
| | [evidence IDA] [pmid 9915832]; |
| | go function: receptor activity [goid 0004872] [evidence |
| | IEA]; |
| | go function: benzodiazepine receptor binding [goid |
| | 0030156] [evidence IPI] [pmid 9915832]; |
| | go_process: biological_process unknown [goid 0000004] |
| | [evidence ND]” |
| | /db_xref=“GeneID:9256” |
| | /db_xref=“LocusID:9256” |

Origin

| | | | | | | | |
|---|---|---|---|---|---|---|---|
| 1 | meqlttlprp | gdpgamepwa | lptwhswtpg | rggepssaap | siadtppaal | qlqelrsees | [SEQ ID NO:21] |
| 61 | skpkgdgssr | pvggtdpega | eaclpslgqq | asssgpacqr | pedeeveafl | kaklnmsfgd | |
| 121 | rpnlellral | gelrqrcail | keenqmlrks | sfpeteekvr | rlkrknaela | viakrleera | |
| 181 | rklqetnlrv | vsaplprpgt | slelcrkala | rqrardlset | asallakdkq | iaalqrecre | |
| 241 | lqarltlvgk | egpqwlhvrd | fdrllresqr | evlrlqrqia | lrnqretlpl | ppswppgpal | |
| 301 | qaragapapg | apgeatpqed | adnlpvilge | pekeqrvqql | eselskkrkk | cesleqeark | |
| 361 | kqrrceelel | qlrqaqnena | rlveensrls | gratekeqve | wenaelrgql | lgvtqerdsa | |
| 421 | lrksqglqsk | lesleqvlkh | mrevaqrrqq | leveheqarl | slrekqeevr | rlqqaqaeaq | |
| 481 | rehegavqll | estldsmqar | vreleeqcrs | qteqfsllaq | elqafrlhpg | pldlltsald | |
| 541 | cgslgdcppp | pcccsipqpc | rgsgpkdldl | ppgspgrctp | kssepapatl | tgvprrtakk | |
| 601 | aeslsnsshs | esihnspksc | ptpevdtase | veeleadsvs | llpaapegsr | ggariqvfla | |
| 661 | rysynpfegp | nenpeaelpl | tageyiyiyg | nmdedgffeg | elmdgrrglv | psnfvervsd | |
| 721 | ddlltslppe | ladlshssgp | elsflsvggg | gsssggqssv | grsqprpeee | dagdelslsp | |
| 781 | speglgeppa | vpyprrlvvl | kqlahsvvla | wepppeqvel | hgfhicvnge | lrqalgpgap | |
| 841 | pkavlenldl | wagplhisvq | altsrgssdp | lrcclavgar | agvvpsqlrv | hrltatsaei | |
| 901 | twvpgnsnla | haiylngeec | ppaspstywa | tfchlrpgtp | yqaqveaqlp | pqgpwepgwe | |
| 961 | rleqraatlq | fttlpagppd | apldvqiepg | pspgiliisw | lpvtidaagt | sngvrvtgya | |
| 1021 | iyadgqkime | vasptagsvl | velsqlqllq | vcrevvvrtm | sphgesadsi | papitpalap | |
| 1081 | aslparvscp | sphpspeara | plasaspgpg | dpssplqhpa | plgtqeppga | ppaspsrema | |
| 1141 | kgshedppap | csqeeagaav | lgtseertas | tstlgekdpg | paapslakqe | aewtageacp | |
| 1201 | assstqgara | qqapntemcq | ggdpgsglrp | raekedtael | gvhlvnslvd | hgrnsdlsdi | |
| 1261 | qeeeeeeeee | eeeelgsrtc | sfqkqvagns | irengaksqp | dpfcetdsde | eileqilelp | |
| 1321 | lqqfcskklf | sipeeeeeee | edeeeeksga | gcssrdpgpp | epallglgcd | sgqprrpgqc | |
| 1381 | plspessrag | dcledmpglv | ggssrrrggg | spekppsrrr | ppdprehcsr | llsnngpqas | |
| 1441 | grlgptrerg | glpviegprt | gleasgrgrl | gpsrrcsrgr | alepglascl | spkcleisie | |
| 1501 | ydsedeqeag | sggisitssc | ypgdreawgt | atvgrprgpp | kansgpkpyp | rlpawekgep | |
| 1561 | errgrsatgr | akeplsrate | tgeargqdgs | grrgpqkrgv | rvlrpstael | vparspsetl | |
| 1621 | ayqhlpvrif | valfdydpvs | mspnpdagee | elpfregqil | kvfgdkdadg | fyqgegggrt | |
| 1681 | gyipcnmvae | vavdspagrq | qllqrgylsp | dillegsgng | pfvystahtt | gpppkprrsk | |
| 1741 | kaesegpaqp | cpgppklvps | adlkaphsmv | aafdynpqes | spnmdveael | pfragdvitv | |
| 1801 | fggmdddgfy | ygelngqrgl | vpsnflegpg | peaggidrep | rtpqaesqrt | rrrrvqc | |

IM. Heterogeneous Nuclear Ribonucleoprotein U (Scaffold Attachment Factor A (hnRNP U Protein)

Heterogeneous Nuclear Ribonucleoprotein U (Scaffold Attachment Factor A) (hnRNP U) protein has been identified as an Akt substrate that interacts with Akt. Human hnRNP U includes isoform a, associated with reference sequence NP_{—}114032 (GenBank Accession No. 14141163) and isoform b, associated with reference sequence NP_{—}004492 (GenBank Accession No. 14141161). A specific sequence identified as an Akt substrate includes a protein similar to hnRNP U (GenBank Accession No. 14044052).

NP_004492. heterogeneous nuclear ribonucleoprotein U isoform b [gi: 14141161]

| | |
|---|---|
| LOCUS | NP_004492 806 aa linear PRI 05-OCT-2003 |
| DEFINITION | heterogeneous nuclear ribonucleoprotein U isoform b; hnRNP U |
| | protein; scaffold attachment factor A; p120 nuclear protein [Homo |
| | sapiens]. |
| ACCESSION | NP_004492 |
| VERSION | NP_004492.2 GI: 14141161 |
| DBSOURCE | REFSEQ: accession NM 004501.2 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 806) |
| AUTHORS | Martens, J. H., Verlaan, M., Kalkhoven, E., Dorsman, J. C. and Zantema, A. |
| TITLE | Scaffold/matrix attachment region elements interact with a |
| | p300-scaffold attachment factor A complex and are bound by |
| | acetylated nucleosomes |
| JOURNAL | Mol. Cell. Biol. 22 (8), 2598-2606 (2002) |
| MEDLINE | 21907223 |
| PUBMED | 11909954 |
| REMARK | GeneRIF: Scaffold/matrix attachment region elements interact with a |
| | p300-scaffold attachment factor A complex and are bound by |
| | acetylated nucleosomes. |
| REFERENCE | 2 (residues 1 to 806) |
| AUTHORS | Davis, M., Hatzubai, A., Andersen, J. S., Ben-Shushan, E., Fisher, G. Z., |
| | Yaron, A., Bauskin, A., Mercurio, F., Mann, M. and Ben-Neriah, Y. |
| TITLE | Pseudosubstrate regulation of the SCF(beta-TrCP) ubiquitin ligase |
| | by hnRNP-U |
| JOURNAL | Genes Dev. 16 (4), 439-451 (2002) |
| MEDLINE | 21838685 |
| PUBMED | 11850407 |
| REMARK | GeneRIF: hnRNP-U engages a highly neddlylated active SCF beta-TRCP |
| | which dissociates in the presence of a high-affinity substrate, |
| | resulting in the ubiquitination of the latter. |
| REFERENCE | 3 (residues 1 to 806) |
| AUTHORS | Kipp, M., Schwab, B. L., Przybylski, M., Nicotera, P. and |
| | Fackelmayer, F. O. |
| TITLE | Apoptotic cleavage of scaffold attachment factor A (SAF-A) by |
| | caspase-3 occurs at a noncanonical cleavage site |
| JOURNAL | J. Biol. Chem. 275 (7), 5031-5036 (2000) |
| MEDLINE | 20138248 |
| PUBMED | 10671544 |
| REFERENCE | 4 (residues 1 to 806) |
| AUTHORS | Gohring, F., Schwab, B. L., Nicotera, P., Leist, M. and Fackelmayer, F. O. |
| TITLE | The novel SAR-binding domain of scaffold attachment factor A |
| (SAF-A) | is a target in apoptotic nuclear breakdown |
| JOURNAL | EMBO J. 16 (24), 7361-7371 (1997) |
| MEDLINE | 98070313 |
| PUBMED | 9405365 |
| REFERENCE | 5 (residues 1 to 806) |
| AUTHORS | Fackelmayer, F. O. and Richter, A. |
| TITLE | Purification of two isoforms of hnRNP-U and characterization of |
| | their nucleic acid binding activity |
| JOURNAL | Biochemistry 33 (34), 10416-10422 (1994) |
| MEDLINE | 94347778 |
| PUBMED | 8068679 |
| REFERENCE | 6 (residues 1 to 806) |
| AUTHORS | Fackelmayer, F. O. and Richter, A. |
| TITLE | hnRNP-U/SAF-A is encoded by two differentially polyadenylated mRNAs |
| | in human cells |
| JOURNAL | Biochim. Biophys. Acta 1217 (2), 232-234 (1994) |
| MEDLINE | 94154006 |
| PUBMED | 7509195 |
| REFERENCE | 7 (residues 1 to 806) |
| AUTHORS | Kiledjian, M. and Dreyfuss, G. |
| TITLE | Primary structure and binding activity of the hnRNP U protein: |
| | binding RNA through RGG box |
| JOURNAL | EMBO J. 11 (7), 2655-2664 (1992) |
| MEDLINE | 92331618 |
| PUBMED | 1628625 |
| COMMENT | REVIEWED REFSEQ: This record has been curated by NCBI staff. The |
| | reference sequence was derived from BC003367.1 and AF068846.1. |
| | On May 17, 2001 this sequence version replaced gi: 4758546. |
| | Summary: This gene belongs to the subfamily of ubiquitously |
| | expressed heterogeneous nuclear ribonucleoproteins (hnRNPs). The |
| | hnRNPs are RNA binding proteins and they complex with heterogeneous |
| | nuclear RNA (hnRNA). These proteins are associated with pre-mRNAs |
| | in the nucleus and appear to influence pre-mRNA processing and |
| | other aspects of mRNA metabolism and transport. While all of the |
| | hnRNPs are present in the nucleus, some seem to shuttle between the |
| | nucleus and the cytoplasm. The hnRNP proteins have distinct nucleic |
| | acid binding properties. The protein encoded by this gene contains |
| | a RNA binding domain and scaffold-associated region (SAR)-specific |
| | bipartite DNA-binding domain. This protein is also thought to be |
| | involved in the packaging of hnRNA into large ribonucleoprotein |
| | complexes. During apoptosis, this protein is cleaved in a |
| | caspase-dependent way. Cleavage occurs at the SALD site, resulting |
| | in a loss of DNA-binding activity and a concomitant detachment of |
| | this protein from nuclear structural sites. But this cleavage does |
| | not affect the function of the encoded protein in RNA metabolism. |
| | Two alternatively spliced transcript variants have been described |
| | for this gene. |
| | Transcript Variant: This variant (2) lacks 54 bases in the coding |
| | region compared to variant 1. This causes the isoform b to be 18 |
| | amino acids shorter than isoform a, but it maintains the same |
| | reading frame. |
| FEATURES | Location/Qualifiers |
| source | 1 . . . 806 |
| | /organism=“Homo sapiens” |
| | /db_xref=“taxon: 9606” |
| | /chromosome=“1” |
| | /map=“1q44” |
| Protein | 1 . . . 806 |
| | /product=“heterogeneous nuclear ribonucleoprotein U |
| | isoform b” |
| | /note=”hnRNP U protein; scaffold attachment factor A; p120 |
| | nuclear protein“ |
| Region | 100 |
| | /region name=”SALD cleavage site“ |
| Region | 328 . . . 444 |
| | /region_name=”SPRY domain. SPRY Domain is named from SPla |
| | and the RY anodine Receptor. Domain of unknown function. |
| | Distant homologues are domains in |
| | butyrophilin/marenostrin/pyrin homologues” |
| | /note=“SPRY” |
| | /db_xref=“CDD:pfam00622” |
| Region | 480 . . . 630 |
| | /region_name=“COG4639, Predicted kinase [General function |
| | prediction only]” |
| | /note=“COG4639” |
| | /db_xref=“CDD:COG4639” |
| variation | 693 |
| | /allele=“F” |
| | /allele=“L” |
| | /db_xref=“dbSNP: 1052660” |
| CDS | 1 . . . 806 |
| | /gene=“HNRPU” |
| | /coded_by=“NM_004501.2:219..2639” |
| | /note=“go component: nucleoplasm [goid 0005654] [evidence |
| | NR]; |
| | go_component: nucleus [goid 0005634] [evidence NR]; |
| | go component: ribonucleoprotein complex [goid 0030529] |
| | [evidence IEA]; |
| | go function: heterogeneous nuclear ribonucleoprotein [goid |
| | 0008436] [evidence TAS] [pmid 7509195]; |
| | go function: RNA binding [goid 0003723] [evidence TAS] |
| | [pmid 1628625]; |
| | go function: DNA binding [goid 0003677] [evidence TAS] |
| | [pmid 1628625]; |
| | go_function: ATP binding [goid 0005524] [evidence IEA]; |
| | go_process: RNA processing [goid 0006396] [evidence TAS] |
| | [pmid 1628625]” |
| | /db_xref=“GeneID:3192” |
| | /db_xref=“LocusID:3192” |
| | /db_xref=“MIM:602869” |

Origin

| | | | | | | | |
|---|---|---|---|---|---|---|---|
| 1 | mssspvnvkk | lkvselkeel | kkrrlsdkgl | kaelmerlqa | alddeeaggr | pamepgngsl | [SEQ ID NO:22] |
| 61 | dlggdsagrs | gagleqeaaa | ggdeeeeeee | eeeegisald | gdqmelgeen | gaagaadsgp | |
| 121 | meeeeaased | engddqgfqe | gedelgdeee | gagdenghge | qqpqppatqq | qqpqqqrgaa | |
| 181 | keaagkssgp | tslfavtvap | pgarqgqqqa | ggdgkteqkg | gdkkrgvkrp | redhgrgyfe | |
| 241 | yieenkysra | kspqppveee | dehfddtvvc | ldtyncdlhf | kisrdrlsas | sitmesfafl | |
| 301 | waggrasygv | skgkvcfemk | vtekipvrhl | ytkdidihev | rigwslttsg | mllgeeefsy | |
| 361 | gyslkgiktc | ncetedygek | fdendvitcf | anfesdevel | syakngqdlg | vafkiskevl | |
| 421 | agrplfphvl | chncavefnf | gqkekpyfpi | peeytfiqnv | pledrvrgpk | gpeekkdcev | |
| 481 | vmmiglpgag | kttwvtkhaa | enpgkynilg | tntimdkmmv | agfkkqmadt | gklntllqra | |
| 541 | pqclgkfiei | aarkkrnfil | dqtnvsaaaq | rrkmclfagf | qrkavvvcpk | dedykqrtqk | |
| 601 | kaevegkdlp | ehavlkmkgn | ftlpevaecf | deityvelqk | eeaqklleqy | keeskkalpp | |
| 661 | ekkqntgskk | snknksgknq | fnrggghrgr | ggfnmrggnf | rggapgnrgg | ynrrgnmpqr | |
| 721 | ggggggsggi | gypyprapvf | pgrgsysnrg | nynrggmpnr | gnynqnfrgr | gnnrgyknqs | |
| 781 | qgynqwqqgq | fwgqkpwsqh | yhqgyy | | | | |

NP_114032. heterogeneous nuclear ribonucloeprotein U isoform a

| | |
|---|---|
| LOCUS | NP_114032 824 aa linear PRI 05-OCT-2003 |
| DEFINITION | heterogeneous nuclear ribonucleoprotein U isoform a; hnRNP U |
| | protein; scaffold attachment factor A; p120 nuclear protein [Homo |
| | sapiens]. |
| ACCESSION | NP_114032 |
| VERSION | NP_114032.1 GI: 14141163 |
| DBSOURCE | REFSEQ: accession NM_031844.1 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 824) |
| AUTHORS | Martens, J. H., Verlaan, M., Kalkhoven, E., Dorsman, J. C. and Zantema, A. |
| TITLE | Scaffold/matrix attachment region elements interact with a |
| | p300-scaffold attachment factor A complex and are bound by |
| | acetylated nucleosomes |
| JOURNAL | Mol. Cell. Biol. 22 (8), 2598-2606 (2002) |
| MEDLINE | 21907223 |
| PUBMED | 11909954 |
| REMARK | GeneRIF: Scaffold/matrix attachment region elements interact with a |
| | p300-scaffold attachment factor A complex and are bound by |
| | acetylated nucleosomes. |
| REFERENCE | 2 (residues 1 to 824) |
| AUTHORS | Davis, M., Hatzubai, A., Andersen, J. S., Ben-Shushan, E., Fisher, G. Z., |
| | Yaron, A., Bauskin, A., Mercurio, F., Mann, M. and Ben-Neriah, Y. |
| TITLE | Pseudosubstrate regulation of the SCF(beta-TrCP) ubiquitin ligase |
| | by hnRNP-U |
| JOURNAL | Genes Dev. 16 (4), 439-451 (2002) |
| MEDLINE | 21838685 |
| PUBMED | 11850407 |
| REMARK | GeneRIF: hnRNP-U engages a highly neddlylated active SCF beta-TRCP |
| | which dissociates in the presence of a high-affinity substrate, |
| | resulting in the ubiquitination of the latter. |
| REFERENCE | 3 (residues 1 to 824) |
| AUTHORS | Kipp, M., Schwab, B. L., Przybylski, M., Nicotera, P. and |
| | Fackelmayer, F. O. |
| TITLE | Apoptotic cleavage of scaffold attachment factor A (SAF-A) by |
| | caspase-3 occurs at a noncanonical cleavage site |
| JOURNAL | J. Biol. Chem. 275 (7), 5031-5036 (2000) |
| MEDLINE | 20138248 |
| PUBMED | 10671544 |
| REFERENCE | 4 (residues 1 to 824) |
| AUTHORS | Gohring, F., Schwab, B. L., Nicotera, P., Leist, M. and Fackelmayer, F.O. |
| TITLE | The novel SAR-binding domain of scaffold attachment factor A |
| | (SAF-A) is a target in apoptotic nuclear breakdown |
| JOURNAL | EMBO J. 16 (24), 7361-7371 (1997) |
| MEDLINE | 98070313 |
| PUBMED | 9405365 |
| REFERENCE | 5 (residues 1 to 824) |
| AUTHORS | Fackelmayer, F. O. and Richter, A. |
| TITLE | Purification of two isoforms of hnRNP-U and characterization of |
| | their nucleic acid binding activity |
| JOURNAL | Biochemistry 33 (34), 10416-10422 (1994) |
| MEDLINE | 94347778 |
| PUBMED | 8068679 |
| REFERENCE | 6 (residues 1 to 824) |
| AUTHORS | Fackelmayer, F. O. and Richter, A. |
| TITLE | hnRNP-U/SAF-A is encoded by two differentially polyadenylated mRNAs |
| | in human cells |
| JOURNAL | Biochim. Biophys. Acta 1217 (2), 232-234 (1994) |
| MEDLINE | 94154006 |
| PUBMED | 7509195 |
| REFERENCE | 7 (residues 1 to 824) |
| AUTHORS | Kiledjian, M. and Dreyfuss, G. |
| TITLE | Primary structure and binding activity of the hnRNP U protein: |
| | binding RNA through RGG box |
| JOURNAL | EMBO J. 11 (7), 2655-2664 (1992) |
| MEDLINE | 92331618 |
| PUBMED | 1628625 |
| COMMENT | REVIEWED REFSEQ: This record has been curated by NCBI staff. The |
| | reference sequence was derived from AF068846.1. |
| | Summary: This gene belongs to the subfamily of ubiquitously |
| | expressed heterogeneous nuclear ribonucleoproteins (hnRNPs). The |
| | hnRNPs are RNA binding proteins and they complex with heterogeneous |
| | nuclear RNA (hnRNA). These proteins are associated with pre-mRNAs |
| | in the nucleus and appear to influence pre-mRNA processing and |
| | other aspects of mRNA metabolism and transport. While all of the |
| | hnRNPs are present in the nucleus, some seem to shuttle between the |
| | nucleus and the cytoplasm. The hnRNP proteins have distinct nucleic |
| | acid binding properties. The protein encoded by this gene contains |
| | a RNA binding domain and scaffold-associated region (SAR)-specific |
| | bipartite DNA-binding domain. This protein is also thought to be |
| | involved in the packaging of hnRNA into large ribonucleoprotein |
| | complexes. During apoptosis, this protein is cleaved in a |
| | caspase-dependent way. Cleavage occurs at the SALD site, resulting |
| | in a loss of DNA-binding activity and a concomitant detachment of |
| | this protein from nuclear structural sites. But this cleavage does |
| | not affect the function of the encoded protein in RNA metabolism. |
| | Two alternatively spliced transcript variants have been described |
| | for this gene. |
| | Transcript Variant: This variant (1) encodes the full length |
| | isoform. |
| FEATURES | Location/Qualifiers |
| source | 1 . . . 824 |
| | /organism=“Homo sapiens” |
| | /db_xref=“taxon:9606” |
| | /chromosome=“1” |
| | /map=“1q44” |
| Protein | 1 . . . 824 |
| | /product=“heterogeneous nuclear ribonucleoprotein U |
| | isoform a” |
| | /note=“hnRNP U protein; scaffold attachment factor A; p120 |
| | nuclear protein” |
| Region | 100 |
| | /region_name=“SALD cleavage site” |
| misc_feature | 213 . . . 230 |
| | /note=“glycine-rich region” |
| Region | 346 . . . 462 |
| | /region_name=“SPRY domain. SPRY Domain is named from SPla |
| | and the RY anodine Receptor. Domain of unknown function. |
| | Distant homologues are domains in |
| | butyrophilin/marenostrin/pyrin homologues” |
| | /note=“SPRY” |
| | /db_xref=“CDD:pfam00622” |
| Region | 498 . . . 648 |
| | /region_name=“COG4639, Predicted kinase [General function |
| | prediction only]” |
| | /note=“COG4639” |
| | /db_xref=“CDD:COG4639” |
| variation | 711 |
| | /allele=“F” |
| | /allele=“L” |
| | /db_xref=“dbSNP:1052660” |
| CDS | 1 . . . 824 |
| | /gene=“HNRPU” |
| | /coded_by=“NM_031844.1:218..2692” |
| | /note=“go_component: nucleoplasm [goid 0005654] [evidence |
| | NR]; |
| | go_component: nucleus [goid 0005634] [evidence NR]; |
| | go_component: ribonucleoprotein complex [goid 0030529] |
| | [evidence IEA]; |
| | go_function: heterogeneous nuclear ribonucleoprotein [goid |
| | 0008436] [evidence TAS] [pmid 7509195]; |
| | go_function: RNA binding [goid 0003723] [evidence TAS] |
| | [pmid 1628625]; |
| | go_function: DNA binding [goid 0003677] [evidence TAS] |
| | [pmid 1628625]; |
| | go_function: ATP binding [goid 0005524] [evidence IEA]; |
| | go_process: RNA processing [goid 0006396] [evidence TAS] |
| | [pmid 1628625]” |
| | /db_xref=“GeneID:3192” |
| | /db_xref=“LocusID:3192” |
| | /db_xref=“MIM:602869” |

Origin

| | | | | | | | |
|---|---|---|---|---|---|---|---|
| 1 | mssspvnvkk | lkvselkeel | kkrrlsdkgl | kaelmerlqa | alddeeaggr | pamepgngsl | [SEQ ID NO:23] |
| 61 | dlggdsagrs | gagleqeaaa | ggdeeeeeee | eeeegisald | gdqmelgeen | gaagaadsgp | |
| 121 | meeeeaased | engddqgfqe | gedelgdeee | gagdenghge | qqpqppatqq | qqpqqqrgaa | |
| 181 | keaagkssgp | tslfavtvap | pgarqgqqqa | ggkkkaeggg | gggrpgapag | dgkteqkggd | |
| 241 | kkrgvkrpre | dhgrgyfeyi | eenkysraks | pqppveeede | hfddtvvcld | tyncdlhfki | |
| 301 | srdrlsassl | tmesfaflwa | ggrasygvsk | gkvcfemkvt | ekipvrhlyt | kdidihevri | |
| 361 | gwslttsgml | lgeeefsygy | slkgiktcnc | etedygekfd | endvitcfan | fesdevelsy | |
| 421 | akngqdlgva | fkiskevlag | rplfphvlch | ncavefnfgq | kekpyfpipe | eytfiqnvpl | |
| 481 | edrvrgpkgp | eekkdcevvm | miglpgagkt | twvtkhaaen | pgkynilgtn | timdkmmvag | |
| 541 | fkkqmadtgk | lntllqrapq | clgkfieiaa | rkkrnfildq | tnvsaaaqrr | kmclfagfqr | |
| 601 | kavvvcpkde | dykqrtqkka | evegkdlpeh | avlkmkgnft | lpevaecfde | ityvelqkee | |
| 661 | aqklleqyke | eskkalppek | kqntgskksn | knksgknqfn | rggghrgrgg | lnmrggnfrg | |
| 721 | gapgnrggyn | rrgnmpqrgg | ggggsggigy | pyprapvfpg | rgsysnrgny | nrggmpnrgn | |
| 781 | ynqnfrgrgn | nrgyknqsqg | ynqwqqgqfw | gqkpwsqhyh | qgyy | | |

IN. Pyruvate Carboxylase

Pyruvate carboxylase has been identified as an Akt substrate that interacts with Akt. Human pyruvate carboxylase is associated with reference sequence NP_{—}000911 (GenBank Accession No. 4505627) and reference sequence NP_{—}0071504 (GenBank Accession No. 11761615). Other relevant sequences include mouse pyruvate carboxylase (GenBank Accession No. 200246).

NP_000911. pyruvate carboxylase [gi: 4505627]

| | |
|---|---|
| LOCUS | NP_000911 1178 aa linear PRI 04-OCT-2003 |
| DEFINITION | pyruvate carboxylase precursor [Homo sapiens]. |
| ACCESSION | NP_000911 |
| VERSION | NP_000911.1 GI: 4505627 |
| DBSOURCE | REFSEQ: accession NM_000920.2 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 1178) |
| AUTHORS | Carbone, M. A. and Robinson, B. H. |
| TITLE | Expression and characterization of a human pyruvate carboxylase |
| | variant by retroviral gene transfer |
| JOURNAL | Biochem. J. 370 (Pt 1), 275-282 (2003) |
| MEDLINE | 22458341 |
| PUBMED | 12437512 |
| REMARK | GeneRIF: expression and characterization of a variant by retroviral |
| | gene transfer |
| REFERENCE | 2 (residues 1 to 1178) |
| AUTHORS | Carbone, M. A., Applegarth, D. A. and Robinson, B. H. |
| TITLE | Intron retention and frameshift mutations result in severe pyruvate |
| | carboxylase deficiency in two male siblings |
| JOURNAL | Hum. Mutat. 20 (1), 48-56 (2002) |
| MEDLINE | 22106101 |
| PUBMED | 12112657 |
| REMARK | GeneRIF: IVS15+2-5delTAGG results in the retention of intron 15 |
| | during pre-mRNA splicing and frameshift mutations. |
| REFERENCE | 3 (residues 1 to 1178) |
| AUTHORS | Jitrapakdee, S. and Wallace, J. C. |
| TITLE | Structure, function and regulation of pyruvate carboxylase |
| JOURNAL | Biochem. J. 340 (Pt 1), 1-16 (1999) |
| MEDLINE | 99247890 |
| PUBMED | 10229653 |
| REFERENCE | 4 (residues 1 to 1178) |
| AUTHORS | Wallace, J. C., Jitrapakdee, S. and Chapman-Smith, A. |
| TITLE | Pyruvate carboxylase |
| JOURNAL | Int. J. Biochem. Cell Biol. 30 (1), 1-5 (1998) |
| MEDLINE | 98260036 |
| PUBMED | 9597748 |
| REFERENCE | 5 (residues 1 to 1178) |
| AUTHORS | Walker, M. E., Baker, E., Wallace, J. C. and Sutherland, G. R. |
| TITLE | Assignment of the human pyruvate carboxylase gene (PC) to 11q13.4 |
| | by fluorescence in situ hybridisation |
| JOURNAL | Cytogenet. Cell Genet. 69 (3-4), 187-189 (1995) |
| MEDLINE | 95212152 |
| PUBMED | 7698008 |
| REFERENCE | 6 (residues 1 to 1178) |
| AUTHORS | Wexler, I. D., Du, Y., Lisgaris, M. V., Mandal, S. K., Freytag, S. O., |
| | Yang, B. S., Liu, T. C, Kwon, M., Patel, M. S. and Kerr, D. S. |
| TITLE | Primary amino acid sequence and structure of human pyruvate |
| | carboxylase |
| JOURNAL | Biochim. Biophys. Acta 1227 (1-2), 46-52 (1994) |
| MEDLINE | 95002202 |
| PUBMED | 7918683 |
| REFERENCE | 7 (residues 1 to 1178) |
| AUTHORS | MacKay, N., Rigat, B., Douglas, C., Chen, H. S. and Robinson, B. H. |
| TITLE | cDNA cloning of human kidney pyruvate carboxylase |
| JOURNAL | Biochem. Biophys. Res. Commun. 202 (2), 1009-1014 (1994) |
| MEDLINE | 94324922 |
| PUBMED | 8048912 |
| REFERENCE | 8 (residues 1 to 1178) |
| AUTHORS | Lamhonwah, A. M., Quan, F. and Gravel, R. A. |
| TITLE | Sequence homology around the biotin-binding site of human |
| | propionyl-CoA carboxylase and pyruvate carboxylase |
| JOURNAL | Arch. Biochem. Biophys. 254 (2), 631-636 (1987) |
| MEDLINE | 87212051 |
| PUBMED | 3555348 |
| REFERENCE | 9 (residues 1 to 1178) |
| AUTHORS | Freytag, S. O. and Collier, K. J. |
| TITLE | Molecular cloning of a cDNA for human pyruvate carboxylase. |
| | Structural relationship to other biotin-containing carboxylases and |
| | regulation of mRNA content in differentiating preadipocytes |
| JOURNAL | J. Biol. Chem. 259 (20), 12831-12837 (1984) |
| MEDLINE | 85030380 |
| PUBMED | 6548474 |
| COMMENT | REVIEWED REFSEQ: This record has been curated by NCBI staff. The |
| | reference sequence was derived from U30891.1 and U30889.1. |
| | Summary: This gene encodes pyruvate carboxylase, which requires |
| | biotin and ATP to catalyse the carboxylation of pyruvate to |
| | oxaloacetate. The active enzyme is a homotetramer arranged in a |
| | tetrahedron which is located exclusively in the mitochondrial |
| | matrix. Pyruvate carboxylase is involved in gluconeogenesis, |
| | lipogenesis, insulin secretion and synthesis of the |
| | neurotransmitter glutamate. Mutations in this gene have been |
| | associated with pyruvate carboxylase deficiency. Two transcript |
| | variants are encoded by this gene. |
| | Transcript Variant: This variant (A) encodes a 5′ UTR which is |
| | longer and different from that found in transcript variant B. Both |
| | variants encode the same protein sequence. |
| FEATURES | Location/Qualifiers |
| source | 1 . . . 1178 |
| | /organism=“Homo sapiens” |
| | /db_xref=“taxon:9606” |
| | /chromosome=“11” |
| | /map=“11q13.4-q13.5” |
| Protein | 1 . . . 1178 |
| | /product=“pyruvate carboxylase precursor” |
| | /EC_number=“6.4.1.1” |
| transit peptide | 1 . . . 20 |
| | /note=“mitochondrial targeting sequence” |
| mat peptide | 21 . . . 1178 |
| | /product=“pyruvate carboxylase” |
| | /EC_number=“6.4.1.1” |
| Region | 35 . . . 1178 |
| | /region_name=“Pyruvate carboxylase [Energy production and |
| | conversion]” |
| | /note=“PycA” |
| | /db_xref=“CDD:COG1038” |
| variation | 76 |
| | /allele=“H” |
| | /allele=“L” |
| | /db_xref=“dbSNP:7104156” |
| variation | 352 |
| | /allele=“A” |
| | /allele=“S” |
| | /db_xref=“dbSNP:1051704” |
| CDS | 1 . . . 1178 |
| | /gene=“PC” |
| | /coded_by=“NM_000920.2:202..3738” |
| | /note=“go_component: mitochondrion [goid 0005739] |
| | [evidence NR]; |
| | go_function: pyruvate carboxylase activity [goid 0004736] |
| | [evidence TAS] [pmid 7918683]; |
| | go_function: biotin binding [goid 0009374] [evidence TAS] |
| | [pmid 8048912]; |
| | go_function: ATP binding [goid 0005524] [evidence TAS] |
| | [pmid 8048912]; |
| | go_function: ligase activity [goid 0016874] [evidence |
| | IEA]; |
| | go_function: manganese ion binding [goid 0030145] |
| | [evidence IEA]; |
| | go_process: biotin metabolism [goid 0006768] [evidence |
| | IEA]; |
| | go_process: gluconeogenesis [goid 0006094] [evidence IEA]; |
| | go_process: metabolism [goid 0008152] [evidence IEA]; |
| | go_process: lipid biosynthesis [goid 0008610] [evidence |
| | IEA]” |
| | /db_xref=“GeneID:5091” |
| | /db_xref=“LocusID:5091” |
| | /db_xref=“MIM:266150” |

Origin

| | | | | | | | |
|---|---|---|---|---|---|---|---|
| 1 | mlkfrtvhgg | lrllgirrts | tapaaspnvr | rleykpikkv | mvanrgeiai | rvfractelg | [SEQ ID NO:24] |
| 61 | irtvaiyseq | dtgqmhrqka | deayligrgl | apvqaylhip | diikvakenn | vdavhpgygf | |
| 121 | lseradfaqa | cqdagvrfig | pspevvrkmg | dkvearaiai | aagvpvvpgt | dapitslhea | |
| 181 | hefsntygfp | iifkaayggg | grgmrvvhsy | eeleenytra | ysealaafgn | galfvekfie | |
| 241 | kprhievqil | gdqygnilhl | yerdcsiqrr | hqkvveiapa | ahldpqlrtr | ltsdsvklak | |
| 301 | qvgyenagtv | eflvdrhgkh | yfievnsrlq | vehtvteeit | dvdlvhaqih | vsegrslpdl | |
| 361 | glrqenirin | gcaiqcrvtt | edparsfqpd | tgrievfrsg | egmgirldna | safqgavisp | |
| 421 | hydsllvkvi | ahgkdhptaa | tkmsralaef | rvrgvktnia | flqnvlnnqq | flagtvdtqf | |
| 481 | idenpelfql | rpaqnraqkl | lhylghvmvn | gpttpipvka | spsptdpvvp | avpigpppag | |
| 541 | frdillregp | egfaravrnh | pglllmdttf | rdahqsllat | rvrthdlkki | apyvahnfsk | |
| 601 | lfsmenwgga | tfdvamrfly | ecpwrrlqel | relipnipfq | mllrganavg | ytnypdnvvf | |
| 661 | kfcevakeng | mdvfrvfdsl | nylpnmllgm | eaagsaggvv | eaaisytgdv | adpsrtkysl | |
| 721 | qyymglaeel | vragthilci | kdmagllkpt | actmlvsslr | drfpdlplhi | hthdtsgagv | |
| 781 | aamlacaqag | advvdvaads | msgmtsqpsm | galvactrgt | pldtevpmer | vfdyseyweg | |
| 841 | arglyaafdc | tatmksgnsd | vyeneipggq | ytnlhfqahs | mglgskfkev | kkayveanqm | |
| 901 | lgdlikvtps | skivgdlaqf | mvqnglsrae | aeaqaeelsf | prsvveflqg | yigvphggfp | |
| 961 | epfrskvlkd | lprvegrpga | sippidiqal | ekelvdrhge | evtpedvlsa | amypdvfahf | |
| 1021 | kdftatfgpl | dslntrlflq | gpkiaeefev | elergktlhi | kalavsdlnr | agqrqvffel | |
| 1081 | ngqlrsilvk | dtqamkemhf | hpkalkdvkg | qigapmpgkv | idikvvagak | vakgqplcvl | |
| 1141 | samkmetvvt | spmegtvrkv | hvtkdmtleg | ddlileie | | | |

NP_071504. pyruvate carboxylase precursor [gi: 11761615]

| | |
|---|---|
| LOCUS | NP_071504 1178 aa linear PRI 05-OCT-2003 |
| DEFINITION | pyruvate carboxylase precursor [Homo sapiens]. |
| ACCESSION | NP_071504 |
| VERSION | NP_071504.1 GI: 11761615 |
| DBSOURCE | REFSEQ: accession NM_022172.1 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 1178) |
| AUTHORS | Carbone, M. A. and Robinson, B. H. |
| TITLE | Expression and characterization of a human pyruvate carboxylase |
| | variant by retroviral gene transfer |
| JOURNAL | Biochem. J. 370 (Pt 1), 275-282 (2003) |
| MEDLINE | 22458341 |
| PUBMED | 12437512 |
| REMARK | GeneRIF: expression and characterization of a variant by retroviral |
| | gene transfer |
| REFERENCE | 2 (residues 1 to 1178) |
| AUTHORS | Carbone, M. A., Applegarth, D. A. and Robinson, B. H. |
| TITLE | Intron retention and frameshift mutations result in severe pyruvate |
| | carboxylase deficiency in two male siblings |
| JOURNAL | Hum. Mutat. 20 (1), 48-56 (2002) |
| MEDLINE | 22106101 |
| PUBMED | 12112657 |
| REMARK | GeneRIF: IVS15+2-5delTAGG results in the retention of intron 15 |
| | during pre-mRNA splicing and frameshift mutations. |
| REFERENCE | 3 (residues 1 to 1178) |
| AUTHORS | Jitrapakdee, S. and Wallace, J. C. |
| TITLE | Structure, function and regulation of pyruvate carboxylase |
| JOURNAL | Biochem. J. 340 (Pt 1), 1-16 (1999) |
| MEDLINE | 99247890 |
| PUBMED | 10229653 |
| REFERENCE | 4 (residues 1 to 1178) |
| AUTHORS | Wallace, J. C., Jitrapakdee, S. and Chapman-Smith, A. |
| TITLE | Pyruvate carboxylase |
| JOURNAL | Int. J. Biochem. Cell Biol. 30 (1), 1-5 (1998) |
| MEDLINE | 98260036 |
| PUBMED | 9597748 |
| REFERENCE | 5 (residues 1 to 1178) |
| AUTHORS | Walker, M. E., Baker, E., Wallace, J. C. and Sutherland, G. R. |
| TITLE | Assignment of the human pyruvate carboxylase gene (PC) to 11q13.4 |
| | by fluorescence in situ hybridisation |
| JOURNAL | Cytogenet. Cell Genet. 69 (3-4), 187-189 (1995) |
| MEDLINE | 95212152 |
| PUBMED | 7698008 |
| REFERENCE | 6 (residues 1 to 1178) |
| AUTHORS | Wexler, I. D., Du, Y., Lisgaris, M. V., Mandal, S. K., Freytag, S. O., |
| | Yang, B. S., Liu, T. C., Kwon, M., Patel, M. S. and Kerr, D. S. |
| TITLE | Primary amino acid sequence and structure of human pyruvate |
| | carboxylase |
| JOURNAL | Biochim. Biophys. Acta 1227 (1-2), 46-52 (1994) |
| MEDLINE | 95002202 |
| PUBMED | 7918683 |
| REFERENCE | 7 (residues 1 to 1178) |
| AUTHORS | MacKay, N., Rigat, B., Douglas, C., Chen, H. S. and Robinson, B. H. |
| TITLE | cDNA cloning of human kidney pyruvate carboxylase |
| JOURNAL | Biochem. Biophys. Res. Commun. 202 (2), 1009-1014 (1994) |
| MEDLINE | 94324922 |
| PUBMED | 8048912 |
| REFERENCE | 8 (residues 1 to 1178) |
| AUTHORS | Lamhonwah, A. M., Quan, F. and Gravel, R. A. |
| TITLE | Sequence homology around the biotin-binding site of human |
| | propionyl-CoA carboxylase and pyruvate carboxylase |
| JOURNAL | Arch. Biochem. Biophys. 254 (2), 631-636 (1987) |
| MEDLINE | 87212051 |
| PUBMED | 3555348 |
| REFERENCE | 9 (residues 1 to 1178) |
| AUTHORS | Freytag, S. O. and Collier, K. J. |
| TITLE | Molecular cloning of a cDNA for human pyruvate carboxylase. |
| | Structural relationship to other biotin-containing carboxylases and |
| | regulation of mRNA content in differentiating preadipocytes |
| JOURNAL | J. Biol. Chem. 259 (20), 12831-12837 (1984) |
| MEDLINE | 85030380 |
| PUBMED | 6548474 |
| COMMENT | REVIEWED REFSEQ: This record has been curated by NCBI staff. The |
| | reference sequence was derived from U30891.1 and U30890.1. |
| | Summary: This gene encodes pyruvate carboxylase, which requires |
| | biotin and ATP to catalyse the carboxylation of pyruvate to |
| | oxaloacetate. The active enzyme is a homotetramer arranged in a |
| | tetrahedron which is located exclusively in the mitochondrial |
| | matrix. Pyruvate carboxylase is involved in gluconeogenesis, |
| | lipogenesis, insulin secretion and synthesis of the |
| | neurotransmitter glutamate. Mutations in this gene have been |
| | associated with pyruvate carboxylase deficiency. Two transcript |
| | variants are encoded by this gene. |
| | Transcript Variant: This variant (2) encodes a 5′ UTR which is |
| | shorter and different from that found in transcript variant 1. Both |
| | variants encode the same protein sequence. |
| FEATURES | Location/Qualifiers |
| source | 1 . . . 1178 |
| | /organism=“Homo sapiens” |
| | /db_xref=“taxon:9606” |
| | /chromosome=“11” |
| | /map=“11q13.4-q13.5” |
| Protein | 1 . . . 1178 |
| | /product=“pyruvate carboxylase precursor” |
| | /EC_number=“6.4.1.1” |
| transit_peptide | 1 . . . 20 |
| | /note=“mitochondrial targeting sequence” |
| mat_peptide | 21 . . . 1178 |
| | /product=“pyruvate carboxylase” |
| | /EC_number=“6.4.1.1” |
| Region | 35 . . . 1178 |
| | /region_name=“Pyruvate carboxylase [Energy production and |
| | conversion]” |
| | /note=“PycA” |
| | /db_xref=“CDD:COG1038” |
| variation | 76 |
| | /allele=“H” |
| | /allele=“L” |
| | /db_xref=“dbSNP:7104156” |
| variation | 352 |
| | /allele=“A” |
| | /allele=“S” |
| | /db_xref=“dbSNP:1051704” |
| CDS | 1 . . . 1178 |
| | /gene=“PC” |
| | /coded_by=“NM_022172.1:132..3668” |
| | /note=“go_component: mitochondrion [goid 0005739] |
| | [evidence NR]; |
| | go_function: pyruvate carboxylase activity [goid 0004736] |
| | [evidence TAS] [pmid 7918683]; |
| | go_function: biotin binding [goid 0009374] [evidence TAS] |
| | [pmid 8048912]; |
| | go_function: ATP binding [goid 0005524] [evidence TAS] |
| | [pmid 8048912]; |
| | go_function: ligase activity [goid 0016874] [evidence |
| | IEA]; |
| | go_function: manganese ion binding [goid 0030145] |
| | [evidence IEA]; |
| | go_process: biotin metabolism [goid 0006768] [evidence |
| | IEA]; |
| | go_process: gluconeogenesis [goid 0006094] [evidence IEA]; |
| | go_process: metabolism [goid 0008152] [evidence IEA]; |
| | go_process: lipid biosynthesis [goid 0008610] [evidence |
| | IEA]” |
| | /db_xref=“GeneID:5091” |
| | /db_xref=“LocusID:5091” |
| | /db_xref=“MIM:266150” |

Origin

| | | | | | | | |
|---|---|---|---|---|---|---|---|
| 1 | mlkfrtvhgg | lrllgirrts | tapaaspnvr | rleykpikkv | mvanrgeiai | rvfractelg | [SEQ ID NO:25] |
| 61 | irtvaiyseq | dtgqmhrqka | deayligrgl | apvqaylhip | diikvakenn | vdavhpgygf | |
| 121 | lseradfaqa | cqdagvrfig | pspevvrkmg | dkvearaiai | aagvpvvpgt | dapitslhea | |
| 181 | hefsntygfp | iifkaayggg | grgmrvvhsy | eeleenytra | ysealaafgn | galfvekfie | |
| 241 | kprhievqil | gdqygnilhl | yerdcsiqrr | hqkvveiapa | ahldpqlrtr | ltsdsvklak | |
| 301 | qvgyenagtv | eflvdrhgkh | yfievnsrlq | vehtvteeit | dvdlvhaqih | vsegrslpdl | |
| 361 | glrqenirin | gcaiqcrvtt | edparsfqpd | tgrievfrsg | egmgirldna | safqgavisp | |
| 421 | hydsllvkvi | ahgkdhptaa | tkmsralaef | rvrgvktnia | flqnvlnnqq | flagtvdtqf | |
| 481 | idenpelfql | rpaqnraqkl | lhylghvmvn | gpttpipvka | spsptdpvvp | avpigpppag | |
| 541 | frdillregp | egfaravrnh | pglllmdttf | rdahqsllat | rvrthdlkki | apyvahnfsk | |
| 601 | lfsmenwgga | tfdvamrfly | ecpwrrlqel | relipnipfq | mllrganavg | ytnypdnvvf | |
| 661 | kfcevakeng | mdvfrvfdsl | nylpnmllgm | eaagsaggvv | eaaisytgdv | adpsrtkysl | |
| 721 | qyymglaeel | vragthilci | kdmagllkpt | actmlvsslr | drfpdlplhi | hthdtsgagv | |
| 781 | aamlacaqag | advvdvaads | msgmtsqpsm | galvactrgt | pldtevpmer | vfdyseyweg | |
| 841 | arglyaafdc | tatmksgnsd | vyeneipggq | ytnlhfqahs | mglgskfkev | kkayveanqm | |
| 901 | lgdlikvtps | skivgdlaqf | mvqnglsrae | aeaqaeelsf | prsvveflqg | yigvphggfp | |
| 961 | epfrskvlkd | lprvegrpga | slppldlqal | ekelvdrhge | evtpedvlsa | amypdvfahf | |
| 1021 | kdftatfgpl | dslntrlflq | gpkiaeefev | elergktlhi | kalavsdlnr | agqrqvffel | |
| 1081 | ngqlrsilvk | dtqamkemhf | hpkalkdvkg | qigapmpgkv | idikvvagak | vakgqplcvl | |
| 1141 | samkmetvvt | spmegtvrkv | hvtkdmtleg | ddlileie | | | |

IO. Eps Domain Containing Protein (RalBP1)

Eps domain containing protein (RalBP1 protein) has been identified as an Akt substrate that interacts with Akt. Human RalBP1 protein is associated with reference sequence NP_{—}114128 (GenBank Accession No. 13994296). Other relevant sequences include mouse RalBP1 protein associated with reference sequence NP_{—}033074 (GenBank Accession No. 6677715).

NP_114128. RAPBP1 associated Eps domain containing 1 [gi: 13994296]

| | |
|---|---|
| LOCUS | NP_114128 744 aa linear PRI 05-OCT-2003 |
| DEFINITION | RALBP1 associated Eps domain containing 1; RALBP1 protein [Homo |
| | sapiens]. |
| ACCESSION | NP_114128 |
| VERSION | NP_114128.1 GI: 13994296 |
| DBSOURCE | REFSEQ: accession NM_031922.1 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 744) |
| AUTHORS | Xu, J., Zhou, Z., Zeng, L., Huang, Y., Zhao, W., Cheng, C., Xu, M., Xie, Y. |
| | and Mao, Y. |
| TITLE | Cloning, expression and characterization of a novel human REPS1 |
| | gene |
| JOURNAL | Biochim. Biophys. Acta 1522 (2), 118-121 (2001) |
| MEDLINE | 21621408 |
| PUBMED | 11750063 |
| COMMENT | PROVISIONAL REFSEQ: This record has not vet been subject to final |
| | NCBI review. The reference sequence was derived from AF251052.1. |
| FEATURES | Location/Qualifiers |
| source | 1 . . . 744 |
| | /organism=“Homo sapiens” |
| | /db_xref=“taxon:9606” |
| | /chromosome=“6” |
| | /map=“6q23.1-q24.1” |
| Protein | 1 . . . 744 |
| | /product=“RALBP1 associated Eps domain containing 1” |
| | /note=“RALBP1 protein” |
| variation | 97 |
| | /allele=“V” |
| | /allele=“G” |
| | /db_xref=“dbSNP:1212987” |
| Region | 226 . . . 317 |
| | /region_name=“Eps15 homology domain” |
| | /note=“EH” |
| | /db_xref=“CDD:smart00027” |
| variation | 665 |
| | /allele=“V” |
| | /allele=“I” |
| | /db_xref=“dbSNP:1044418” |
| variation | 710 |
| | /allele=“W” |
| | /allele=“L” |
| | /db_xref=“dbSNP:1730376” |
| CDS | 1 . . . 744 |
| | /gene=“REPS1” |
| | /coded by=“NM_031922.1:94..2328” |
| | /db_xref=“GeneID:85021” |
| | /db_xref=“LocusID:85021” |

Origin

| | | | | | | | |
|---|---|---|---|---|---|---|---|
| 1 | melcgatrlg | yfgrsqfyia | lklvavaqsg | fplrvesint | vkdlplprfv | askneqesrh | [SEQ ID NO:26] |
| 61 | aasyssdsen | qgsysgvipp | ppgrgqvkkg | svshdtvqpr | tsadaqepas | pvvspqqspp | |
| 121 | tsphtwrkhs | rhpsggnser | plagpgpfws | pfgeaqsgss | agdavwsghs | ppppqenwvs | |
| 181 | fadtpptstl | ltmhpasvqd | qttvrtvasa | ttaieirrqs | ssyddpwkit | deqrqyyvnq | |
| 241 | fktiqpdlng | fipgsaakef | ftksklpile | lshiwelsdf | dkdgaltlde | fcaafhlvva | |
| 301 | rkngydlpek | lpeslmpkli | dledsadvgd | qpgevgysgs | paeappsksp | smpslnqtwp | |
| 361 | elnqsseqwe | tfserssssq | tltqfdsnia | padpdtaivh | pvpirmtpsk | ihmqemelkr | |
| 421 | tgsdhtnpts | pllvkpsdll | eenkinssvk | fasgntvadg | ysssdsftsd | peqigsnvtr | |
| 481 | qrshsgtspd | ntappppppr | pqpshsrsss | ldmnrtftvt | tgqqqagvva | hppavpprpq | |
| 541 | psqapgpavh | rpvdadglit | htstspqqip | eqpnfvdfsq | fevfaasnvn | deqddeaekh | |
| 601 | pevlpaekas | dpasslrvak | tdskteekta | asapanvskg | ttplapppkp | vrrrlksede | |
| 661 | lrpevdehtq | ktgvlaavla | sqpsiprsvg | kdkkaiqasi | rrnketntvl | arlnselqqq | |
| 721 | lkdvleeris | levqleqlrp | fshl | | | | |

IP. Nonmuscle myosin IIA (NMMIIA)

Nonmuscle myosin IIA (NMMIIA) has been identified as an Akt substrate that interacts with Akt. Human NMMIIA is associated with reference sequence NP_{—}002464 (GenBank Accession No. 12667788). Other relevant sequences include mouse NMMIIA associated with reference sequence NP_{—}071855 (GenBank Accession No. 20137006).

NP_002464. myosin, heavy polypeptide 9, non-muscle [gi: 12667788]

| | |
|---|---|
| LOCUS | NP_002464 1960 aa linear PRI 06-OCT-2003 |
| DEFINITION | myosin, heavy polypeptide 9, non-muscle [Homo sapiens]. |
| ACCESSION | NP_002464 |
| VERSION | NP_002464.1 GI: 12667788 |
| DBSOURCE | REFSEQ: accession NM_002473.2 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 1960) |
| AUTHORS | Lamant, L., Gascoyne, R. D., Duplantier, M. M., Armstrong, F., Raghab, A., |
| | Chhanabhai, M., Rajcan-Separovic, E., Raghab, J., Delsol, G. and |
| | Espinos, E. |
| TITLE | Non-muscle myosin heavy chain (MYH9): a new partner fused to ALK in |
| | anaplastic large cell lymphoma |
| JOURNAL | Genes Chromosomes Cancer 37 (4), 427-432 (2003) |
| MEDLINE | 22683269 |
| PUBMED | 12800156 |
| REMARK | GeneRIF: In a case of anaplastic large cell lymphoma, a portion of |
| | MYH9 is found to be fused to the ALK gene in a novel chromosomal |
| | abnormality, t(2; 22)(p23; q11.2). |
| REFERENCE | 2 (residues 1 to 1960) |
| AUTHORS | Deutsch, S., Rideau, A., Bochaton-Piallat, M. L., Merla, G., Geinoz, A., |
| | Gabbiani, G., Schwede, T., Matthes, T., Antonarakis, S. E. and Beris, P. |
| TITLE | Asp1424Asn MYH9 mutation results in an unstable protein responsible |
| | for the phenotypes in May-Hegglin anomaly/Fechtner syndrome |
| JOURNAL | Blood 102 (2), 529-534 (2003) |
| MEDLINE | 22718684 |
| PUBMED | 12649151 |
| REMARK | GeneRIF: The Asp1424Asn mutation in the MYH9 gene causes the |
| | May-Hegglin anomaly, Fechtner syndrome, Sebastian syndrome, & |
| | Epstein syndrome, which result from a highly unstable protein & |
| | failure to reorganize the megakaryocyte cytoskeleton for platelet |
| | production. |
| REFERENCE | 3 (residues 1 to 1960) |
| AUTHORS | Seri, M., Pecci, A., Di Bari, F., Cusano, R., Savino, M., Panza, E., |
| | Nigro, A., Noris, P., Gangarossa, S., Rocca, B., Gresele, P., |
| | Bizzaro, N., Malatesta, P., Koivisto, P. A., Longo, I., Musso, R., |
| | Pecoraro, C., Iolascon, A., Magrini, U., Rodriguez Soriano, J., |
| | Renieri, A., Ghiggeri, G. M., Ravazzolo, R., Balduini, C. L. and |
| | Savoia, A. |
| TITLE | MYH9-related disease: May-Hegglin anomaly, Sebastian syndrome, |
| | Fechtner syndrome, and Epstein syndrome are not distinct entities |
| | but represent a variable expression of a single illness |
| JOURNAL | Medicine (Baltimore) 82 (3), 203-215 (2003) |
| MEDLINE | 22676762 |
| PUBMED | 12792306 |
| REMARK | GeneRIF: MYH9 gene is implicated in May-Hegglin anomaly, Sebastian |
| | syndrome, Fechtner syndrome and Epstein syndrome which are |
| | autosomal dominant macrothrombocytopenias. |
| REFERENCE | 4 (residues 1 to 1960) |
| | AUTHORS Mhatre, A. N., Kim, Y., Brodie, H. A. and Lalwani, A. K. |
| TITLE | Macrothrombocytopenia and progressive deafness is due to a mutation |
| | in MYH9 |
| JOURNAL | Otol Neurotol 24 (2), 205-209 (2003) |
| MEDLINE | 22508750 |
| PUBMED | 12621333 |
| REMARK | GeneRIF: A single base pair transition in MYH9, resulting in an |
| | amino acid substitution D1424N, is responsible for |
| | macrothrombocytopenia and hearing loss in the kindred under study. |
| REFERENCE | 5 (residues 1 to 1960) |
| AUTHORS | Ghiggeri, G. M., Caridi, G., Magrini, U., Sessa, A., Savoia, A., Seri, M., |
| | Pecci, A., Romagnoli, R., Gangarossa, S., Noris, P., Sartore, S., |
| | Necchi, V., Ravazzolo, R. and Balduini, C. L. |
| TITLE | Genetics, clinical and pathological features of glomerulonephritis |
| | associated with mutations of nonmuscle myosin IIA (Fechtner |
| | syndrome) |
| JOURNAL | Am. J. Kidney Dis. 41 (1), 95-104 (2003) |
| MEDLINE | 22386817 |
| PUBMED | 12500226 |
| REMARK | GeneRIF: A major role is indicated for the NMMHC-IIA abnormality in |
| | the pathogenesis of leukocyte, platelet, and kidney defects in |
| | Fechtner syndrome. |
| REFERENCE | 6 (residues 1 to 1960) |
| AUTHORS | Rey, M., Vicente-Manzanares, M., Viedma, F., Yanez-Mo, M., |
| | Urzainqui, A., Barreiro, O., Vazquez, J. and Sanchez-Madrid, F. |
| TITLE | Cutting edge: association of the motor protein nonmuscle myosin |
| | heavy chain-IIA with the C terminus of the chemokine receptor CXCR4 |
| | in T lymphocytes |
| JOURNAL | J. Immunol. 169 (10), 5410-5414 (2002) |
| MEDLINE | 22309087 |
| PUBMED | 12421915 |
| REMARK | GeneRIF: Motor protein nonmuscle myosin heavy chain-IIA and CXCR$ |
| | colocalize at the leading edge of migrating T lymphocytes, together |
| | with filamentous actin and myosin light chain. |
| REFERENCE | 7 (residues 1 to 1960) |
| AUTHORS | Seri, M., Savino, M., Bordo, D., Cusano, R., Rocca, B., Meloni, I., Di |
| | Bari, F., Koivisto, P. A., Bolognesi, M., Ghiggeri, G. M., Landolfi, R., |
| | Balduini, C. L., Zelante, L., Ravazzolo, R., Renieri, A. and Savoia, A. |
| TITLE | Epstein syndrome: another renal disorder with mutations in the |
| | nonmuscle myosin heavy chain 9 gene |
| JOURNAL | Hum. Genet. 110 (2), 182-186 (2002) |
| MEDLINE | 21932559 |
| PUBMED | 11935325 |
| REMARK | GeneRIF: mutation in epstein syndrome |
| REFERENCE | 8 (residues 1 to 1960) |
| AUTHORS | Heath, K. E., Campos-Barros, A., Toren, A., Rozenfeld-Granot, G., |
| | Carlsson, L. E., Savige, J., Denison, J. C., Gregory, M. C., White, J. G., |
| | Barker, D. F., Greinacher, A., Epstein, C. J., Glucksman, M. J. and |
| | Martignetti, J. A. |
| TITLE | Nonmuscle myosin heavy chain IIA mutations define a spectrum of |
| | autosomal dominant macrothrombocytopenias: May-Hegglin anomaly and |
| | Fechtner, Sebastian, Epstein, and Alport-like syndromes |
| JOURNAL | Am. J. Hum. Genet. 69 (5), 1033-1045 (2001) |
| MEDLINE | 21473756 |
| PUBMED | 11590545 |
| REMARK | GeneRIF: mutations cause a spectrum of autosomal dominant |
| | macrothrombocytopenias: May-Hegglin anomaly and Fechtner, |
| | Sebastian, Epstein, and Alport-like syndromes; hence, the name |
| | ‘MYHIIA syndrome’ is proposed to encompass all of these disorders |
| REFERENCE | 9 (residues 1 to 1960) |
| AUTHORS | Toothaker, L. E., Gonzalez, D. A., Tung, N., Lemons, R. S., Le Beau, M. M., |
| | Arnaout, M. A., Clayton, L. K. and Tenen, D. G. |
| TITLE | Cellular myosin heavy chain in human leukocytes: isolation of 5′ |
| | cDNA clones, characterization of the protein, chromosomal |
| | localization, and upregulation during myeloid differentiation |
| JOURNAL | Blood 78 (7), 1826-1833 (1991) |
| MEDLINE | 92003925 |
| PUBMED | 1912569 |
| REFERENCE | 10 (residues 1 to 1960) |
| AUTHORS | Simons, M., Wang, M., McBride, O. W., Kawamoto, S., Yamakawa, K., |
| | Gdula, D., Adelstein, R. S. and Weir, L. |
| TITLE | Human nonmuscle myosin heavy chains are encoded by two genes |
| | located on different chromosomes |
| JOURNAL | Circ. Res. 69 (2), 530-539 (1991) |
| MEDLINE | 91316803 |
| PUBMED | 1860190 |
| REFERENCE | 11 (residues 1 to 1960) |
| AUTHORS | Saez, C. G., Myers, J. C., Shows, T. B. and Leinwand, L. A. |
| TITLE | Human nonmuscle myosin heavy chain mRNA: generation of diversity |
| | through alternative polyadenylylation |
| JOURNAL | Proc. Natl. Acad. Sci. U.S.A. 87 (3), 1164-1168 (1990) |
| MEDLINE | 90138958 |
| PUBMED | 1967836 |
| REFERENCE | 12 (residues 1 to 1960) |
| AUTHORS | Lee, C. L. and Atassi, M. Z. |
| TITLE | Enzymic and immunochemical properties of lysozyme. Accurate |
| | definition of the antigenic site around the disulphide bridge |
| | 30-115 (site 3) by ‘surface-simulation’ synthesis |
| JOURNAL | Biochem. J. 167 (3), 571-581 (1977) |
| MEDLINE | 78103135 |
| PUBMED | 603622 |
| COMMENT | PROVISIONAL REFSEQ: This record has not yet been subject to final |
| NCBI | review. The reference sequence was derived from Z82215.1. |
| FEATURES | Location/Qualifiers |
| source | 1 . . . 1960 |
| | /organism=“Homo sapiens” |
| | /db_xref=“taxon:9606” |
| | /chromosome=“22” |
| | /map=“22q13.1” |
| Protein | 1 . . . 1960 |
| | /product=“myosin, heavy polypeptide 9, non-muscle” |
| Region | 83 . . . 764 |
| | /region_name=“Myosin head (motor domain)” |
| | /note=“myosin head” |
| | /db_xref=“CDD:pfam00063” |
| Region | 1091 . . . 1924 |
| | /region_name=“Myosin tail. The myosin molecule is a |
| | multi-subunit complex made up of two heavy chains and four |
| | light chains it is a fundamental contractile protein found |
| | in all eukaryote cell types. This family consists of the |
| | coiled-coil myosin heavy chain tail region. The |
| | coiled-coil is composed of the tail from two molecules of |
| | myosin. These can then assemble into the macromolecular |
| | thick filament. The coiled-coil region provides the |
| | structural backbone the thick filament” |
| | /note=“Myosin tail” |
| | /db_xref=“CDD:pfam01576” |
| variation | 1626 |
| | /allele=“V” |
| | /allele=“I” |
| | /db_xref=“dbSNP:2269529” |
| CDS | 1 . . . 1960 |
| | /gene=“MYH9” |
| | /coded_by=“NM_002473.2:1..5883” |
| | /note=“match: proteins: Tr:Q63731 Sw:P35579 Tr:O93522 |
| | Tr:Q62812 Sw:P14105 Sw:P10587 Tr:O08638 Sw:P35748 |
| | Tr:O08639 Tr:O94944 Tr:Q02015; |
| | go_component: non-muscle myosin [goid 0005860] [evidence |
| | TAS] [pmid 1967836]; |
| | go_component: kinesin complex [goid 0005871] [evidence |
| | IEA]; |
| | go_component: myosin [goid 0016459] [evidence IEA]; |
| | go_function: adenosinetriphosphatase activity [goid |
| | 0004002] [evidence NR] [pmid 1967836]; |
| | go_function: motor activity [goid 0003774] [evidence NR]; |
| | go_function: actin binding [goid 0003779] [evidence IEA]; |
| | go_function: ATP binding [goid 0005524] [evidence IEA]; |
| | go_function: calmodulin binding [goid 0005516] [evidence |
| | IEA]; |
| | go_process: hearing [goid 0007605] [evidence IEA]; |
| | go_process: protein amino acid alkylation [goid 0008213] |
| | [evidence IEA]” |
| | /db_xref=“GeneID:4627” |
| | /db_xref=“LocusID:4627” |
| | /db_xref=“MIM:160775” |

Origin

| [SEQ ID NO:27] | |
|---|---|
| 1 | maqqaadkyl yvdknfinnp laqadwaakk lvwvpsdksg fepaslkeev geeaivelve |
| 61 | ngkkvkvnkd diqkmnppkf skvedmaelt clneasvlhn lkeryysgli ytysglfcvv |
| 121 | inpyknlpiy seeivemykg kkrhempphi yaitdtayrs mmqdredqsi lctgesgagk |
| 181 | tentkkviqy layvasshks kkdqgelerq llqanpilea fgnaktvknd nssrfgkfir |
| 241 | infdvngyiv ganietylle ksrairqake ertfhifyyl lsgagehlkt dlllepynky |
| 301 | rflsnghvti pgqqdkdmfq etmeamrimg ipeeeqmgll rvisgvlqlg nivfkkernt |
| 361 | dqasmpdnta aqkvshllgi nvtdftrgil tprikvgrdy vqkaqtkeqa dfaiealaka |
| 421 | tyermfrwlv lrinkaldkt krqgasfigi ldiagfeifd lnsfeqlcin ytneklqqlf |
| 481 | nhtmfileqe eyqregiewn fidfgldlqp cidliekpag ppgilallde ecwfpkatdk |
| 541 | sfvekvmqeq gthpkfqkpk qlkdkadfci ihyagkvdyk adewlmknmd plndniatll |
| 601 | hqssdkfvse lwkdvdriig ldqvagmset alpgafktrk gmfrtvgqly keqlaklmat |
| 661 | lrntnpnfvr ciipnhekka gkldphlvld qlrcngvleg iricrqgfpn rvvfqefrqr |
| 721 | yeiltpnsip kgfmdgkqac vlmikaleld snlyrigqsk vffragvlah leeerdlkit |
| 781 | dviigfqacc rgylarkafa krqqqltamk vlqrncaayl klrnwqwwrl ftkvkpllqv |
| 841 | srqeeemmak eeelvkvrek qlaaenrlte metlqsqlma eklqlqeqlq aetelcaeae |
| 901 | elrarltakk qeleeichdl earveeeeer cqhlqaekkk mqqniqelee qleeeesarq |
| 961 | klqlekvtte aklkkleeeq iiledqnckl akekklledr iaefttnlte eeekskslak |
| 1021 | lknkheamit dleerlrree kqrqelektr rklegdstdl sdqiaelqaq iaelkmqlak |
| 1081 | keeelqaala rveeeaaqkn malkkirele sqiselqedl eserasrnka ekqkrdlgee |
| 1141 | lealkteled tldstaaqqe lrskreqevn ilkktleeea ktheaqiqem rqkhsqavee |
| 1201 | laeqleqtkr vkanlekakq tlenergela nevkvllqgk gdsehkrkkv eaqlqelqvk |
| 1261 | fnegervrte ladkvtklqv eldnvtglls qsdskssklt kdfsalesql qdtqellqee |
| 1321 | nrqklslstk lkqvedekns freqleeeee akhnlekqia tlhaqvadmk kkmedsvgcl |
| 1381 | etaeevkrkl qkdleglsqr heekvaaydk lektktrlqq elddllvdld hqrqsacnle |
| 1441 | kkqkkfdqll aeektisaky aeerdraeae areketkals laraleeame qkaelerlnk |
| 1501 | qfrtemedlm sskddvgksv helekskral eqqveemktq leeledelqa tedaklrlev |
| 1561 | nlqamkaqfe rdlqgrdeqs eekkkqlvrq vremeaeled erkqrsmava arkklemdlk |
| 1621 | dleahidsan knrdeaikql rklqaqmkdc mrelddtras reeilaqake nekklksmea |
| 1681 | emiqlqeela aaerakrqaq qerdeladei anssgkgala leekrrlear iaqleeelee |
| 1741 | eqgntelind rlkkanlqid qintdlnler shaqknenar qqlerqnkel kvklqemegt |
| 1801 | vkskykasit aleakiaqle eqldnetker qaackqvrrt ekklkdvllq vdderrnaeq |
| 1861 | ykdqadkast rlkqlkrqle eaeeeaqran asrrklqrel edatetadam nrevsslknk |
| 1921 | lrrgdlpfvv prrmarkgag dgsdeevdgk adgaeakpae |

IQ. Stress 70 Protein (P66 mot1/GRP75)

Stress 70 Protein (P66 mot1/GRP75) has been identified as an Akt substrate that interacts with Akt. Human P66 mot1/GRP75 is associated with reference sequence NP_{—}004125 (GenBank Accession No. 24234688). Other relevant sequences include mouse P66 mot1/GRP75 (GenBank Accession No. 6754256).

NP_004125. heat shock 70kDa protein 9B precursor [gi: 24234688]

| | |
|---|---|
| LOCUS | NP_004125 679 aa linear PRI 05-OCT-2003 |
| DEFINITION | heat shock 70kDa protein 9B precursor; heat shock 70 kD protein 9; |
| | stress-70 protein, mitochondrial; 75 kDa glucose regulated protein; |
| | peptide-binding protein 74; mortalin, perinuclear; p66-mortalin |
| | [Homo sapiens]. |
| ACCESSION | NP_004125 |
| VERSION | NP_004125.3 GI: 24234688 |
| DBSOURCE | REFSEQ: accession NM_004134.3 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 679) |
| AUTHORS | Kaul, S. C, Yaguchi, T., Taira, K., Reddel, R. R. and Wadhwa, R. |
| TITLE | Overexpressed mortalin (mot-2)/mthsp70/GRP75 and hTERT cooperate to |
| | extend the in vitro lifespan of human fibroblasts |
| JOURNAL | Exp. Cell Res. 286 (1), 96-101 (2003) |
| MEDLINE | 22615561 |
| PUBMED | 12729798 |
| REMARK | GeneRIF: Results demonstrate that mot-2 and telomerase can |
| | cooperate in the immortalization process. |
| REFERENCE | 2 (residues 1 to 679) |
| AUTHORS | Wadhwa, R., Yaguchi, T., Hasan, M. K., Mitsui, Y., Reddel, R. R. and |
| | Kaul, S. C. |
| TITLE | Hsp70 family member, mot-2/mthsp70/GRP75, binds to the cytoplasmic |
| | sequestration domain of the p53 protein |
| JOURNAL | Exp. Cell Res. 274 (2), 246-253 (2002) |
| MEDLINE | 21898287 |
| PUBMED | 11900485 |
| REMARK | GeneRIF: Cytoplasmic sequestration and inactivation of p53 by mot-2 |
| | occurs by its binding to the cytoplasmic sequestration |
| | domain; perturbation of mot-p53 interactions can be employed to |
| | abrogate cytoplasmic retention of wild-type p53 in tumors. |
| REFERENCE | 3 (residues 1 to 679) |
| AUTHORS | Carette, J., Lehnert, S. and Chow, T. Y. |
| TITLE | Implication of PBP74/mortalin/GRP75 in the radio-adaptive response |
| JOURNAL | Int. J. Radiat. Biol. 78 (3), 183-190 (2002) |
| MEDLINE | 21859237 |
| PUBMED | 11869473 |
| REMARK | GeneRIF: Implication of PBP74/mortalin/GRP75 in the radio-adaptive |
| | response. |
| REFERENCE | 4 (residues 1 to 679) |
| AUTHORS | Kaul, S. C., Wadhwa, R., Matsuda, Y., Hensler, P. J., Pereira-Smith, O. M., |
| | Komatsu, Y. and Mitsui, Y. |
| TITLE | Mouse and human chromosomal assignments of mortalin, a novel member |
| | of the murine hsp70 family of proteins |
| JOURNAL | FEBS Lett. 361 (2-3), 269-272 (1995) |
| MEDLINE | 95212562 |
| PUBMED | 7698336 |
| REFERENCE | 5 (residues 1 to 679) |
| AUTHORS | Bhattacharyya, T., Karnezis, A. N., Murphy, S. P., Hoang, T., |
| | Freeman, B. C., Phillips, B. and Morimoto, R. I. |
| TITLE | Cloning and subcellular localization of human mitochondrial hsp70 |
| JOURNAL | J. Biol. Chem. 270 (4), 1705-1710 (1995) |
| MEDLINE | 95130547 |
| PUBMED | 7829505 |
| REFERENCE | 6 (residues 1 to 679) |
| AUTHORS | Domanico, S. Z., DeNagel, D. C., Dahlseid, J. N., Green, J. M. and |
| | Pierce, S. K. |
| TITLE | Cloning of the gene encoding peptide-binding protein 74 shows that |
| | it is a new member of the heat shock protein 70 family |
| JOURNAL | Mol. Cell. Biol. 13 (6), 3598-3610 (1993) |
| MEDLINE | 93268309 |
| PUBMED | 7684501 |
| COMMENT | REVIEWED REFSEQ: This record has been curated by NCBI staff. The |
| | reference sequence was derived from BC024034.1 and AU130219.1. |
| | On Oct 22, 2002 this sequence version replaced gi: 21314627. |
| | Summary: The product encoded by this gene belongs to the heat shock |
| | protein 70 family which contains both heat-inducible and |
| | constitutively expressed members. The latter are called heat-shock |
| | cognate proteins. This gene encodes a heat-shock cognate protein. |
| | This protein plays a role in the control of cell proliferation. It |
| | may also act as a chaperone. |
| FEATURES | Location/Qualifiers |
| source | 1 . . . 679 |
| | /organism=“Homo sapiens” |
| | /db_xref=“taxon:9606” |
| | /chromosome=“5” |
| | /map=“5q31.1” |
| Protein | 1 . . . 679 |
| | /product=“heat shock 70kDa protein 9B precursor” |
| | /note=“heat shock 70kD protein 9; stress-70 protein, |
| | mitochondrial; 75 kDa glucose regulated protein; |
| | peptide-binding protein 74; mortalin, perinuclear; |
| | p66-mortalin” |
| transit_peptide | 1 . . . 51 |
| | /note=“mitochondrial targeting sequence” |
| mat_peptide | 52 . . . 679 |
| | /product=“heat shock 70kDa protein 9B” |
| Region | 55 . . . 653 |
| | /region_name=“Hsp70 protein. Hsp70 chaperones help to fold |
| | many proteins. Hsp70 assisted folding involves repeated |
| | cycles of substrate binding and release. Hsp70 activity is |
| | ATP dependent. Hsp70 proteins are made up of two regions: |
| | the amino terminus is the ATPase domain and the carboxyl |
| | terminus is the substrate binding region” |
| | /note=“HSP70” |
| | /db_xref=“CDD:pfam00012” |
| CDS | 1 . . . 679 |
| | /gene=“HSPA9B” |
| | /coded_by=“NM_004134.3:94..2133” |
| | /note=“go_component: cytoplasm [goid 0005737] [evidence E] |
| | [pmid 7684501]; |
| | go_component: mitochondrion [goid 0005739] [evidence TAS] |
| | [pmid 7829505]; |
| | go_function: ATP binding [goid 0005524] [evidence IEA]” |
| | /db_xref=“GeneID:3313” |
| | /db_xref=“LocusID:3313” |
| | /db_xref=“MIM:600548” |

Origin

| [SEQ ID NO:28] | |
|---|---|
| 1 | misasraaaa rlvgaaasrg ptaarhqdsw nglsheafrl vsrrdyasea ikgavvgidl |
| 61 | gttnscvavm egkqakvlen aegarttpsv vaftadgerl vgmpakrqav tnpnntfyat |
| 121 | krligrrydd pevqkdiknv pfkivrasng dawveahgkl yspsqigafv lmkmketaen |
| 181 | ylghtaknav itvpayfnds qrqatkdagq isglnvlrvi neptaaalay gldksedkvi |
| 241 | avydlgggtf disileiqkg vfevkstngd tflggedfdq allrhivkef kretgvdltk |
| 301 | dnmalqrvre aaekakcels ssvqtdinlp yltmdssgpk hlnmkltraq fegivtdlir |
| 361 | rtiapcqkam qdaevsksdi gevilvggmt rmpkvqqtvq dlfgrapska vnpdeavaig |
| 421 | aaiqggvlag dvtdvllldv tplslgietl ggvftklinr nttiptkksq vfstaadgqt |
| 481 | qveikvcqge remagdnkll gqftligipp aprgvpqiev tfdidangiv hvsakdkgtg |
| 541 | reqqiviqss gglskddien mvknaekyae edrrkkerve avnmaegiih dtetkmeefk |
| 601 | dqlpadecnk lkeeiskmre llarkdsetg enirqaassl qqaslklfem aykkmasere |
| 661 | gsgssgtgeq kedqkeekq |

II. Screening Assays:

According to the invention, the following assays may be used to identify compounds that modulate interaction (e.g., binding) of Akt or bioactive fragments thereof with Akt substrates or bioactive fragments thereof and hence, insulin response modulators. Such insulin response modulators are particularly useful in regulation of phosphatidylinositol 3-kinase, regulation of insulin signaling to GLUT4, regulation of insulin signaling to glycogen synthase kinase, regulation of intracellular GLUT4 trafficking and regulation of intracellular retention of GLUT4. The assays feature identifying modulators of the activity of Akt substrates or bioactive fragments thereof, including, but not limited to, those activities identified in Table 1 and subsections IA-IQ. In certain embodiments, the assays of the present invention feature identifying compounds that modulate the phosphorylation state of Akt substrates.

The assays of the present invention are used to identify Akt modulators of the activity of Akt or bioactive fragments thereof or Akt substrates or bioactive fragments thereof. The modulators of the present invention are particularly useful in modulating insulin related activities but can also affect non-insulin related activities. In various embodiments, the modulators may affect activities in non-insulin responsive tissues and cells.

IIA. Cell Free Assays

In one embodiment, an assay of the present invention is a cell-free assay in which a composition comprising assay reagents (e.g., an Akt substrate polypeptide, Akt polypeptide or biologically active portions thereof), is contacted with a test compound and the ability of the test compound to modulate binding of the Akt substrate polypeptide to the Akt polypeptide (or bioactive fragments thereof) is determined. Binding of the Akt substrate or Akt (or bioactive fragments thereof) can be accomplished, for example, by coupling the polypeptide or fragment with a radioisotope or enzymatic label such that binding of polypeptide reagents can be determined by detecting the labeled compound or polypeptide in a complex. For example, test compounds or polypeptides can be labeled with ¹²⁵I, ³⁵S, ¹⁴C, or ³H, either directly or indirectly, and the radioisotope detected by direct counting of radioemmission or by scintillation counting. Alternatively, polypeptides can be enzymatically labeled with, for example, horseradish peroxidase, alkaline phosphatase, or luciferase, and the enzymatic label detected by determination of conversion of an appropriate substrate to product.

Determination of binding of reagents can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon of surface plasmon resonance (SPR) can be used as an indication of real-time reactions between biological molecules.

In a preferred embodiment, the assay includes contacting Akt polypeptide or biologically active portion thereof with an Akt target molecule, e.g., an akt substrate or a bioactive fragment thereof to form an assay mixture, contacting the assay mixture with a test compound, and determining the ability of the test compound to interact with the Akt polypeptide, wherein determining the ability of the test compound to interact with the Akt polypeptide comprises determining the ability of the test compound to preferentially bind to Akt or the bioactive portion thereof as compared to the Akt target molecule (e.g., an Akt substrate). In another embodiment, the assay includes contacting the Akt substrate polypeptide or biologically active portion thereof with an Akt substrate target molecule, e.g., Akt or a bioactive fragment thereof to form an assay mixture, contacting the assay mixture with a test compound, and determining the ability of the test compound to modulate binding between the Akt substrate polypeptide and the Akt polypeptide.

In another embodiment, the assay is a cell-free assay in which a composition comprising an Akt polypeptide and an Akt substrate polypeptide (or bioactive portions thereof) is contacted with a test compound and the ability of the test compound to modulate (e.g., stimulate or inhibit) the activity of the Akt polypeptide or Akt substrate polypeptide (or biologically active portions thereof) is determined.

Determining the ability of the test compound to modulate the activity of an Akt or an Akt substrate polypeptide can be accomplished, for example, by determining the ability of the Akt substrate polypeptide to modulate the activity of a downstream binding partner or target molecule by one of the methods described herein for cell-based assays. For example, the catalytic/enzymatic activity of the target molecule on an appropriate downstream substrate can be determined as previously described.

In yet another embodiment, the cell-free assay involves contacting an Akt substrate polypeptide or biologically active portion thereof with an Akt substrate target molecule that binds the Akt substrate polypeptide to form an assay mixture, contacting the assay mixture with a test compound, and determining the ability of the test compound (e.g., Akt) to preferentially modulate the activity of an Akt substrate binding partner or target molecule, as compared to the Akt substrate.

In more than one embodiment of the above assay methods of the present invention, it may be desirable to immobilize either the Akt substrate or Akt (or target molecules) to facilitate separation of complexed from uncomplexed forms of one or both of the proteins, as well as to accommodate automation of the assay. The ability of a test compound to modulate Akt substrate polypeptide activity, Akt polypeptide activity, interaction of an Akt substrate polypeptide with an Akt polypeptide (or target interaction or activity) in the presence and absence of a candidate compound, can be accomplished in any vessel suitable for containing the reactants. Examples of such vessels include microtitre plates, test tubes, and micro-centrifuge tubes. In one embodiment, a fusion protein can be provided so as to add a domain that allows one or both of the proteins to be bound to a matrix. For example, glutathione-S-transferase/Akt substrate fusion proteins, glutathione-S-transferase/Akt fusion proteins, or target fusion proteins can be adsorbed onto glutathione sepharose beads (Sigma Chemical, St. Louis, Mo.) or glutathione derivatized microtitre plates, which are then combined with the test compound or the test compound and either the non-adsorbed Akt polypeptide or Akt substrate polypeptide (or target polypeptide), and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of Akt substrate binding or activity or Akt binding or activity (or target binding or activity) determined using standard techniques.

Additional exemplary Akt and/or Akt substrate fusion proteins (or target fusion proteins) include, but are not limited to, chitin binding domain (CBD) fusion proteins, hemagglutinin epitope tagged (HA)-fusion proteins, His fusion proteins (e.g., His₆ tagged proteins), FLAG tagged fusion proteins, AU1 tagged proteins, and the like.

Other techniques for immobilizing proteins on matrices can also be used in the screening assays of the invention. For example, either an Akt polypeptide, an Akt substrate polypeptide or target polypeptide can be immobilized utilizing conjugation of biotin and streptavidin. Biotinylated Akt polypeptide, Akt substrate polypeptide or target polypeptide can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with Akt polypeptide, Akt substrate polypeptide or target polypeptide but which do not interfere with binding of the Akt substrate polypeptide to Akt polypeptide (or substrate to target binding) can be derivatized to the wells of the plate, and unbound Akt or Akt substrate polypeptide (or target) trapped in the wells by antibody conjugation. Methods for detecting such complexes, in addition to those described above for the GST-immobilized complexes, include immunodetection of complexes using antibodies reactive with the Akt substrate polypeptide, Akt polypeptide or target polypeptide, as well as enzyme-linked assays which rely on detecting an enzymatic activity associated with the Akt substrate polypeptide, Akt polypeptide or target polypeptide.

In one aspect of the invention, the Akt substrate or Akt polypeptides can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with Akt substrate or Akt (“binding proteins” or “target molecules”) and are involved in Akt substrate or Akt activity. Such target molecules are also likely to be involved in the regulation of cellular activities modulated by the Akt substrate polypeptides or Akt polypeptides.

At least one exemplary two-hybrid system is based on the modular nature of most transcription factors, which consist of separable DNA-binding and activation domains. Briefly, the assay utilizes two different DNA constructs. In one construct, the gene that codes for a first polypeptide (the “bait” polypeptide, e.g., Akt or Akt substrate) is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene that encodes the protein that interacts with the bait polypeptide.

Another exemplary two-hybrid system, referred to in the art as the CytoTrap™ system, is based in the modular nature of molecules of the Ras signal transduction cascade. Briefly, the assay features a fusion protein comprising the “bait” protein and Son-of-Sevenless (SOS) and the cDNAs for unidentified proteins (the “prey”) in a vector that encodes myristylated target proteins. Expression of an appropriate bait-prey combination results in translocation of SOS to the cell membrane where it activates Ras. Cytoplasmic reconstitution of the Ras signaling pathway allows identification of proteins that interact with the bait protein of interest, for example, an Akt or Akt substrate protein. Additional mammalian two hybrid systems are also known in the art and can be utilized to identify Akt or Akt substrate interacting proteins. Moreover, at least one of the above-described assays can be utilized to identify Akt-interacting domains or regions of the Akt substrate protein or alternativey, to identify Akt substrate-interacting domain or regions of the Akt protein.

IIB. Cell Based Assays

In one embodiment, an assay is a cell-based assay in which a cell capable of expressing a Akt substrate polypeptide, or biologically active portion thereof, is contacted with a test compound and the ability of the test compound to modulate the expression of the Akt substrate polypeptide, or biologically active portion thereof, determined. In another embodiment, an assay is a cell-based assay in which a cell which expresses an Akt substrate polypeptide or Akt polypeptide (or biologically active portions thereof) is contacted with a test compound and the ability of the test compound to modulate the activity of the Akt substrate polypeptide or Akt polypeptide (or biologically active portions thereof) determined. The cell, for example, can be of mammalian origin or a yeast cell. The polypeptides, for example, can be expressed heterologously or native to the cell. Determining the ability of the test compound to modulate the activity of an Akt substrate or Akt polypeptide (or biologically active portions thereof) can be accomplished by assaying for any of the activities of an Akt substrate or Akt polypeptide described herein. Determining the ability of the test compound to modulate the activity of an Akt substrate polypeptide or Akt polypeptide (or biologically active portions thereof) can also be accomplished by assaying for the activity of an Akt substrate target molecule. In one embodiment, determining the ability of the test compound to modulate the activity of an Akt substrate polypeptide, or biologically active portion thereof, is accomplished by assaying for the ability to bind Akt or a bioactive portion thereof. In another embodiment, determining the ability of the test compound to modulate the activity of an Akt substrate polypeptide, or biologically active portion thereof, is accomplished by assaying for the activity of Akt (e.g., by assaying for GLUT4 trafficking). In a preferred embodiment, the cell overexpresses the Akt substrate polypeptide, or biologically active portion thereof, and optionally, overexpresses Akt, or biologically active portion thereof. In another preferred embodiment, the cell expresses Akt, or biologically active portion thereof. In yet another preferred example, the cell is contacted with a compound that stimulates an Akt substrate-associated activity or Akt-associated activity (e.g., insulin) and the ability of a test compound to modulate the Akt substrate-associated activity is determined.

As used herein, the term “bioactive” fragment includes any portion (e.g., a segment of contiguous amino acids) of an Akt substrate or Akt protein sufficient to exhibit or exert at least one Akt substrate- or Akt-associated activity including, for example, the ability to bind to Akt or Akt substrate, respectively. In various embodiments, the Akt may be one of two isoforms, Akt1 or Akt2. In another embodiment, the bioactive peptide is derived from the amino acid sequence of Akt. In another embodiment, the bioactive peptide corresponds to a fragment or domain as set forth in subsections IA-IQ, supra or a smaller bioactive fragment thereof. In another embodiment, the bioactive peptide is derived from an Akt substrate and can include, for example, amino acid residues sufficient to effect enzymatic activity.

According to the cell-based assays of the present invention, determining the ability of the test compound to modulate the activity of the Akt polypeptide or biologically active portion thereof, can be determined by assaying for any of the native activities of an Akt polypeptide as described herein. Moreover, the activity of Akt, can be determined by assaying for an indirect activity which is coincident to the activity of Akt. For example, the effect of the test compound on the ability of an Akt-expressing cell to uptake glucose in an insulin-dependent manner can be assayed in the presence of the test compound. Furthermore, determining the ability of the test compound to modulate the activity of the Akt and/or Akt substrate polypeptide or biologically active portion thereof, can be determined by assaying for an activity which is not native to the Akt substrate or Akt polypeptide, but for which the cell has been recombinantly engineered. For example, the cell can be engineered to express a target molecule which is a recombinant protein comprising a bioactive portion of Akt operatively linked to a non-Akt polypeptide. It is also intended that in preferred embodiments, the cell-based assays of the present invention comprise a final step of identifying the compound as a modulator of Akt substrate activity or Akt activity.

III. Assay Reagents

IIIA. Test Compounds

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In a preferred embodiment, the library is a natural product library.

IIIB. Antibodies, Bioactive Fragments and Fusion Proteins

Preferred aspects of the invention feature Akt polypeptides, Akt substrate polypeptides and biologically active portions (i.e., bioactive fragments) of Akt polypeptides or Akt substrate polypeptides, including polypeptide fragments suitable for use in making Akt substrate or Akt fusion proteins. In one embodiment, Akt polypeptides or Akt substrate polypeptides can be isolated from cells or tissue sources by an appropriate purification scheme using standard protein purification techniques. Akt polypeptide or Akt substrate polypeptides can be further derived from said isolated polypeptides using standard enzymatic techniques. In another embodiment, Akt substrate polypeptides, Akt polypeptides or bioactive fragments thereof are produced by recombinant DNA techniques. Alternative to recombinant expression, Akt substrate polypeptides, Akt polypeptides or bioactive fragments thereof can be synthesized chemically using standard peptide synthesis techniques.

Polypeptides of the invention are preferably “isolated” or “purified”. The terms “isolated” and “purified” are used interchangeably herein. “Isolated” or “purified” means that the protein or polypeptide is substantially free of cellular material or other contaminating proteins from the cell or tissue source from which the polypeptide is derived, substantially free of other protein fragments, for example, non-desired fragments in a digestion mixture, or substantially free from chemical precursors or other chemicals when chemically synthesized. The language “substantially free of cellular material” includes preparations in which the polypeptide is separated from other components of the cells from which it is isolated or recombinantly produced. In one embodiment, the language “substantially free of cellular material” includes preparations of polypeptide having less than about 30% (by dry weight) of non-Akt substrate or non-Akt polypeptide (also referred to herein as a “contaminating protein”), more preferably less than about 20% of non-Akt substrate or non-Akt polypeptide, still more preferably less than about 10% of non-Akt substrate or non-Akt polypeptide, and most preferably less than about 5% non-Akt substrate or non-Akt polypeptide. When the polypeptide or protein is recombinantly produced, it is also preferably substantially free of culture medium, i.e., culture medium represents less than about 20%, more preferably less than about 10%, and most preferably less than about 5% of the volume of the polypeptide preparation. When the polypeptide or protein is produced by, for example, chemical or enzymatic processing from isolated or purified Akt substrate or Akt protein, the preparation is preferably free of enzyme reaction components or chemical reaction components and is free of non-desired Akt substrate or Akt fragments, i.e., the desired polypeptide represents at least 75% (by dry weight) of the preparation, preferably at least 80%, more preferably at least 85%, and even more preferably at least 90%, 95%, 99% or more or the preparation.

The language “substantially free of chemical precursors or other chemicals” includes preparations of polypeptide in which the polypeptide is separated from chemical precursors or other chemicals which are involved in the synthesis of the polypeptide. In one embodiment, the language “substantially free of chemical precursors or other chemicals” includes preparations having less than about 30% (by dry weight) of chemical precursors or reagents, more preferably less than about 20% chemical precursors or reagents, still more preferably less than about 10% chemical precursors or reagents, and most preferably less than about 5% chemical precursors or reagents.

Bioactive fragments of Akt substrate or Akt include polypeptides comprising amino acid sequences sufficiently identical to or derived from the amino acid sequence of the Akt substrate protein or the Akt protein, respectively, which include less amino acids than the full length protein, and exhibit at least one biological activity of the full-length protein. Typically, biologically active portions comprise a domain or motif with at least one activity of the full-length protein. A biologically active portion of an Akt substrate or Akt polypeptide can be a polypeptide which is, for example, 10, 20, 30, 40, 50, 100, 150, 200, 250, 300, 350, 400, 450, 500, 550, 600, 650, 700, 750, 800, 850, 900, 950, 1000 or more amino acids in length. In a preferred embodiment, a bioactive portion of an Akt protein comprises a portion comprising an Akt substrate interacting domain. Moreover, other biologically active portions, in which other regions of the protein are deleted, can be prepared by recombinant techniques and evaluated for one or more of the functional activities of a native Akt substrate or Akt protein. Mutants of Akt and/or Akt substrate can also be utilized as assay reagents, for example, mutants having reduced, enhanced or otherwise altered biological properties identified according to one of the activity assays described herein.

As defined herein, an Akt polypeptide or Akt substrate polypeptide of the invention includes polypeptides having the amino acid sequences set forth in subsections IA-IQ, infra, as well as homologs an/or orthologues of said polypeptides, i.e. polypeptides having sufficient sequence identity to function in the same manner as the described polypeptides. To determine the percent identity of two amino acid sequences (or of two nucleotide or amino acid sequences), the sequences are aligned for optimal comparison purposes (e.g., gaps can be introduced in the first sequence or second sequence for optimal alignment). The amino acid residues or nucleotides at corresponding amino acid positions or nucleotide positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10. BLAST alignments can be generated and percent identity calculated using BLAST protein searches (e.g., the XBLAST program) using Akt substrate, Akt or a portion thereof as a query, score=50, wordlength=3.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Research 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The invention also provides Akt substrates and Akt chimeric or fusion proteins. As used herein, an Akt substrate or Akt “chimeric protein” or “fusion protein” comprises an Akt substrate or Akt polypeptide operatively linked to a non-Akt substrate polypeptide or non-Akt polypeptide, respectively. A “Akt substrate polypeptide” or “Akt polypeptide” refers to a polypeptide having an amino acid sequence corresponding to the Akt substrate or Akt protein, respectively, whereas a “non-Akt substrate polypeptide” or “non-Akt polypeptide” refers to a polypeptide having an amino acid sequence corresponding to a protein which is not substantially identical to the Akt substrate protein or Akt protein. Within a fusion protein the Akt substrate or Akt polypeptide can correspond to all or a portion of an Akt substrate or Akt protein. In a preferred embodiment, an Akt substrate or Akt fusion protein comprises at least one biologically active portion of an Akt substrate or Akt protein, respectively. In another preferred embodiment, an Akt substrate or Akt fusion protein comprises at least two biologically active portions of an Akt substrate or Akt protein, respectively. In yet another preferred embodiment, a fusion protein can comprise Akt substrate, or a bioactive portion thereof, operatively linked to Akt, or a bioactive portion thereof, such that Akt substrate and Akt, or their respective bioactive portions are brought into close proximity. Within the fusion protein, the term “operatively linked” is intended to indicate that the Akt substrate or Akt polypeptide and the non-Akt substrate polypeptide or non-Akt polypeptide are fused in-frame to each other. The non-Akt substrate polypeptide or non-Akt polypeptide can be fused to the N-terminus or C-terminus of the Akt substrate polypeptide or Akt polypeptide, respectively.

For example, in one embodiment, the fusion protein is a GST-fusion protein in which the Akt substrate or Akt sequences are fused to the C-terminus of the GST sequences. In another embodiment, the fusion protein is a chitin binding domain (CBD) fusion protein in which the Akt substrate or Akt sequences are fused to the N-terminus of chitin binding domain (CBD) sequences. Such fusion proteins can facilitate the purification of recombinant Akt substrate or Akt.

Preferably, a chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety. An Akt substrate- or Akt-encoding nucleic acid can be cloned into such an expression vector such that the fusion moiety is linked in-frame to the Akt substrate or Akt polypeptide.

An Akt substrate polypeptide or Akt polypeptide, or a portion or fragment of Akt substrate or Akt, can also be used as an immunogen to generate antibodies that bind Akt substrate or Akt or that block Akt substrate/Akt binding using standard techniques for polyclonal and monoclonal antibody preparation. A full-length polypeptide can be used or, alternatively, the invention provides antigenic peptide fragments for use as immunogens. Preferably, an antigenic fragment comprises at least 8 amino acid residues of the amino acid sequence of an Akt substrate or Akt and encompasses an epitope of Akt substrate or Akt such that an antibody raised against the peptide forms a specific immune complex with Akt substrate or Akt, respectively. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of Akt substrate or Akt that are located on the surface of the protein, e.g., hydrophilic regions. Antigenic determinants at the termini of Akt substrate are preferred for the development of antibodies that do not interfere with the Akt substrate:Akt interaction. Alternatively, interfering antibodies can be generated towards antigenic determinants located within the Akt interacting domain of Akt substrate. The latter are preferred for therapeutic purposes.

An Akt substrate or Akt immunogen typically is used to prepare antibodies by immunizing a suitable subject, (e.g., rabbit, goat, mouse or other mammal) with the immunogen. An appropriate immunogenic preparation can contain, for example, recombinantly expressed Akt substrate or Akt polypeptide or a chemically synthesized Akt substrate or Akt polypeptide. The preparation can further include an adjuvant, such as Freund's complete or incomplete adjuvant, or similar immunostimulatory agent. Immunization of a suitable subject with an immunogenic Akt substrate or Akt preparation induces a polyclonal anti-Akt substrate or anti-Akt antibody response, respectively.

Accordingly, another aspect of the invention pertains to anti-Akt substrate or anti-Akt antibodies. The term “antibody” as used herein refers to immunoglobulin molecules and immunologically active portions of immunoglobulin molecules, i.e., molecules that contain an antigen binding site which specifically binds (immunoreacts with) an antigen, such as Akt substrate or Akt. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind Akt substrate. The term “monoclonal antibody” or “monoclonal antibody composition”, as used herein, refers to a population of antibody molecules that contain only one species of an antigen binding site capable of immunoreacting with a particular epitope of Akt substrate or Akt. A monoclonal antibody composition thus typically displays a single binding affinity for a particular Akt substrate or Akt polypeptide with which it immunoreacts.

Polyclonal anti-Akt substrate or anti-Akt antibodies can be prepared as described above by immunizing a suitable subject with an Akt substrate or Akt immunogen, respectively. The antibody titer in the immunized subject can be monitored over time by standard techniques, such as with an enzyme linked immunosorbent assay (ELISA) using immobilized Akt substrate or Akt. If desired, the antibody molecules can be isolated from the mammal (e.g., from the blood) and further purified by well known techniques, such as protein A chromatography to obtain the IgG fraction. At an appropriate time after immunization, e.g., when the anti-Akt substrate or anti-Akt antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an Akt substrate or Akt immunogen as described above, and the culture supematants of the resulting hybridoma cells are screened to identify a hybridoma producing a monoclonal antibody that binds Akt substrate or Akt, respectively.

Any of the many well known protocols used for fusing lymphocytes and immortalized cell lines can be applied for the purpose of generating an anti-Akt substrate monoclonal antibody (see, e.g., G. Galfre et al. (1977) Nature 266:55052; Gefter et al. Somatic Cell Genet., cited supra; Lemer, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind Akt substrate or Akt, e.g., using a standard ELISA assay.

Alternative to preparing monoclonal antibody-secreting hybridomas, a monoclonal anti-Akt substrate or anti-Akt antibody can be identified and isolated by screening a recombinant combinatorial immunoglobulin library (e.g., an antibody phage display library) with Akt substrate or Akt to thereby isolate immunoglobulin library members that bind Akt substrate or Akt, respectively. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

An anti-Akt substrate or anti-Akt antibody (e.g., monoclonal antibody) can be used to isolate Akt substrate or Akt, bioactive portions thereof, or fusion proteins by standard techniques, such as affinity chromatography or immunoprecipitation. Anti-Akt antibodies or anti-Akt substrate antibodies made according to any of the above-described techniques can be used to detect protein levels in donor or acceptor fractions as part of certain assay methodologies described herein. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³I, ³⁵S or ³H.

IIIC. Recombinant Expression Vectors and Assay Cells

Another aspect of the invention pertains to vectors, preferably expression vectors, for producing the proteins reagents of the instant invention. As used herein, the term “vector” refers to a nucleic acid molecule capable of transporting another nucleic acid to which it has been linked. A preferred vector is a “plasmid”, which refers to a circular double stranded DNA loop into which additional DNA segments can be ligated. In the present specification, “plasmid” and “vector” can be used interchangeably as the plasmid is the most commonly used form of vector.

The recombinant expression vectors of the invention comprise a nucleic acid that encodes, for example substrate or Akt or a bioactive fragment or Akt substrate or bioactive fragment, in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). The expression vectors can be introduced into host cells to thereby produce proteins, including fusion proteins or peptides. Alternatively, retroviral expression vectors and/or adenoviral expression vectors can be utilized to express the proteins of the present invention.

The recombinant expression vectors of the invention can be designed for expression of Akt substrate or Akt polypeptides in prokaryotic or eukaryotic cells. For example, Akt substrate or Akt polypeptides can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990).

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Purified fusion proteins are particularly useful in the cell-free assay methodologies of the present invention.

In yet another embodiment, a substrate or Akt-encoding or Akt-substrate-encoding nucleic acid is expressed in mammalian cells, for example, for use in the cell-based assays described herein. When used in mammalian cells, the expression vector's control functions are often provided by viral regulatory elements. In another embodiment, the recombinant mammalian expression vector is capable of directing expression of the nucleic acid preferentially in a particular cell type (e.g., tissue-specific regulatory elements are used to express the nucleic acid).

Another aspect of the invention pertains to assay cells into which a recombinant expression vector has been introduced. An assay cell can be prokaryotic or eukaryotic, but preferably is eukaryotic. A preferred assay cell is an adipocyte or muscle, for example, a human adipocyte cell, a human muscle cell, an adipocyte cell line (e.g., murine 3T3-L1 cells), or a muscle cell line (e.g., murine C2 or C2C12 cells or rat C6 cells). Human adipocytes can be derived from human adipose tissue as undifferentiated cells and expanded ex vivo prior to differentiation for use in the assays of the present invention. Cell lines are cultured according to art-recognized techniques. Vector DNA can be introduced into prokaryotic or eukaryotic cells via conventional transformation or transfection techniques. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals. An assay cell of the invention, can be contacted with a test compound and assayed for any Akt substrate and/or Akt biological activity in order to identify the compound as an insulin responsive modulator. Biological activities that can further be assayed as part of the methodologies of the present invention include, but are not limited to, (1) modulation of cellular protein degradation; (2) modulation of fat metabolism; (3) modulation of insulin clearance; (4) modulation of proteosome activation; (5) modulation of ubiquitination; and (6) peroxisome targeting activity. Biological activities that can also be assayed as part of the methodologies of the present invention include, but are not limited to, (1) interaction between Akt substrate or a bioactive fragment thereof with Akt or a bioactive fragment thereof; (2) modulation of vesicle translocation; (3) modulation of GLUT4 translocation; (4) regulation of intracellular trafficking; (5) regulation of glucose uptake; and (6) regulation of phosphatidylinositol 3-kinase pathway components; and/or regulation of insulin signaling. In addition, other biological activities which may be assayed for include those listed in Table 1 and/or subsections IA-IQ, supra.

IV. Pharmaceutical Compositions

This invention further pertains to insulin response modulators identified by the above-described screening assays. Insulin response modulators identified by the above-described screening assays can be tested in an appropriate animal model. For example, an insulin response modulator identified as described herein can be used in an animal model to determine the efficacy, toxicity, or side effects of treatment with such a modulator. Alternatively, a modulator identified as described herein can be used in an animal model to determine the mechanism of action of such an agent. Furthermore, this invention pertains to uses of insulin response modulators identified by the above-described screening assays for therapeutic treatments as described infra.

Accordingly, the insulin response modulators of the present invention can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

V. Methods of Treatment

The present invention also features methods of treatment or therapeutic methods. In one embodiment, the invention features a method of treating a subject (e.g., a human subject in need thereof) with a modulatory compound identified according to the present invention, such that a desired therapeutic effect is achieved. In another embodiment, the method involves administering to an isolated tissue or cell line from the subject a modulatory compound identified according to the methodology described herein, such that a desired therapeutic effect is achieved. In a preferred embodiment, the invention features a method of treating a subject having an insulin response disorder, for example, reduced insulin sensitivity or insulin resistance or diabetes (e.g., Type II diabetes). The present invention also provides for therapeutic methods of treating a subject having pre-diabetes or symptoms thereof, hyperglycemia and/or Type I diabetes. Desired therapeutic effects include a modulation of any Akt substrate-, Akt- or Akt substrate/Akt-associated activity, as described herein. A preferred therapeutic effect is modulation of glucose uptake and/or transport. Desired therapeutic effects also include, but are not limited to curing or healing the subject, alleviating, relieving, altering or ameliorating a disease or disorder in the subject or at least one symptom of said disease or disorder in the subject, or otherwise improving or affecting the health of the subject. A preferred aspect of the invention pertains to methods of modulating Akt substrate/Akt interactions for therapeutic purposes.

The modulators identified by the methods disclosed herein may be used in a subject to modulate insulin responsiveness, regulate glucose transport, regulate gluconeogenesis, regulate glucose homeostasis, and to regulate blood glucose levels.

The effectiveness of treatment of a subject with an insulin response modulator can be accomplished by (i) detecting the level of insulin responsiveness or, alternatively, glucose tolerance in the subject prior to treating with an appropriate modulator; (ii) detecting the level of insulin responsiveness or, alternatively, glucose tolerance in the subject prior post treatment with the modulator; (iii) comparing the levels pre-administration and post administration; and (iv) altering the administration of the modulator to the subject accordingly. For example, increased administration of the modulator may be desirable if the subject continues to demonstrate insensitive insulin responsiveness.

Alternatively, the effectiveness of treatment of a subject with an insulin response modulator can be accomplished by (i) detecting the blood glucose or glucose tolerance in the subject prior to treating with an appropriate modulator; (ii) detecting the blood glucose level or, alternatively, glucose tolerance in the subject prior post treatment with the modulator; (iii) comparing the levels pre-administration and post administration; and (iv) altering the administration of the modulator to the subject accordingly. For example, increased or sustained administration of the modulator may be desirable if the subject fails to adequately clear blood glucose.

VI. Diagnostic Assays

The present invention is based at least in part on the discovery that the Akt substrates identified above and Akt interact and are proposed to be involved in regulating insulin responsiveness in a subject. Based on the proposed role for Akt substrate:Akt in normal insulin responsiveness, aberrant Akt substrate:Akt interaction, expression and/or activity may be associated with abnormal insulin responsiveness. Accordingly, the present invention also features diagnostic assays, for determining aberrant Akt substrate:Akt interaction, expression or activity, in the context of a biological sample (e.g., blood, serum, cells, tissue) to thereby determine whether an individual is afflicted with a disease or disorder (e.g., abnormal insulin responsiveness), or is at risk of developing such a disorder. The invention also provides for prognostic (or predictive) assays for determining whether an individual is at risk of developing such a disorder (e.g., a disorder associated with aberrant Akt substrate expression or activity). Such assays can be used for prognostic or predictive purpose to thereby phophylactically treat an individual prior to the onset of a disease or disorder. A preferred agent for detecting an Akt substrate or Akt protein is an antibody capable of binding to substrate or Akt, respectively, preferably an antibody with a detectable label. The term “biological sample” is intended to include tissues, cells and biological fluids isolated from a subject, as well as tissues, cells and fluids present within a subject. The invention also encompasses kits for the detection of aberrant Akt substrate:Akt interaction, expression or activity in a biological sample. For example, the kit can comprise a labeled compound or agent capable of detecting Akt substrate and/or Akt in a biological sample; means for determining the amount of Akt substrate and/or Akt in the sample; and/or means for comparing the amount of Akt substrate in the sample with a standard. The compound or agent can be packaged in a suitable container. The kit can further comprise instructions for using the kit.

This invention is further illustrated by the following examples which should not be construed as limiting. The contents of all references, patents and published patent applications cited throughout this application are hereby incorporated by reference.

### EXEMPLIFICATION

### EXAMPLE 1

### Insulin Signaling Through Akt/Protein Kinase B Analyzed by Small Interfering RNA Mediated Gene Silencing

In order to determine the importance of Akt1 and Akt2, the following experiment seeks to selectively inhibit the expression of Akt protein kinases in intact cultured adipocytes through the use of interference RNA. This powerful approach overcomes the problems encountered in mouse gene knockouts where loss of both Akt1 and Akt2 genes is lethal. First discovered in Caenorhabditis elegans (Fire et al. (1998) Nature 391:806-811), this gene-silencing technique uses double-stranded RNA to activate nuclease-containing protein complexes (RNA-induced silencing complex) to target a specific MRNA species, which is then degraded (Hammond et al. (2001) Nature 404:293-296; Hammond et al. (2001) Science 293:1146-1150). Before insertion into protein-silencing complexes, processing of double-stranded RNA into small interfering RNA (siRNA) duplexes of 21-23 nt occurs by enzymes known as Dicers (Bernstein et al. (2001) Nature 409:363-366; Zamore et al. (2000) Cell 101:25-33; Elbashir et al. (2000) Genes Dev. 15:188-200). Extension of the technique to mammalian cells has involved the use of synthetic siRNA duplexes of 21-base lengths transfected directly into cultured cells, where decreased levels of selected proteins can be observed in response to siRNA after 24-72 h (Elbashir et al. (2001) Nature 411:494-498). The following example demonstrates the details of a method that can be used successfully to silence genes in insulin-sensitive cultured adipocytes and to show that virtual complete ablation of Akt1 and ≈70% depletion of Akt2 can be achieved. Combined depletion of these Akt isoforms largely attenuates insulin signaling to both GLUT4 glucose transporters and glycogen synthase kinase (GSK)-3, demonstrating an obligatory role of Akt protein kinases in these insulin-signaling cascades.

Materials and Methods

Materials. Human insulin was obtained from Eli Lilly (Indianapolis, Ind.). Goat polyclonal anti-Akt1 Ab (antigen human Akt1 peptide near C terminus, sc-7126), horseradish peroxidase-conjugated donkey anti-goat IgG, mouse monoclonal anti-lamin A/C (sc7293), monoclonal anti-GSK-3α/β, and polyclonal anti-protein kinase C (PKC)λ/ξ (C-20, sc216) were from Santa Cruz Biotechnology (Santa Cruz, Calif.). Rabbit polyclonal anti-Akt2 Ab (antigen peptide at C-terminal of human Akt2) was provided by the University of Pennsylvania (Philadelphia, Pa.). Rabbit polyclonal Ab against adipocyte complement-related protein of 30 kDa (Acrp30) was from Affinity BioReagents (Golden, Colo.) and Ab against nonmuscle myosin IIB was from Covance (Richmond, Calif.). Polyclonal Abs against phospho-Akt Thr-308/309, phospho-GSK-α/β (Ser-21/9), and phospho-Erk-1/2 were from Cell Signaling Technology (Beverly, Mass.). Monoclonal phosphotyrosine Ab (4G10) was from Upstate USA (Charlottesville, Va.). The FITC-conjugated goat anti-mouse was from BioSource International (Camarillo, Calif.). The plasmid expressing Myc-tagged GLUT4-EGFP was constructed as described in Jiang et al. (2002) J. Biol. Chem. 277:509-515.

Design and Synthesis of siRNA Duplexes. The 21-mer sense and antisense strands of RNA oligonucleotides were designed as described in Elbashir et al. (2001) Nature 411:494-498. The RNA oligonucleotides were synthesized in the 2′-ACE(™) protected form, which enhances overall RNA stability and resistance to nucleases. The complementary sense and antisense strands of RNA oligonucleotides were mixed, 2′-deprotected, annealed, and purified by PAGE. Gel-purified duplexes were subsequently desalted by using reverse-phase column chromatography, followed by washing with 75% ethanol twice to ensure complete salt removal and dried by use of a Speed-Vac. The pellets were resuspended in nuclease-free water before transfection into cultured cells.

Cell Culture and Electroporation of 3T3L1 Adipocytes. The 3T3-L1 fibroblasts were grown in DMEM supplemented with 10% FBS, 50 μg/ml streptomycin, and 50 units/ml penicillin and differentiated into adipocytes as described in Harrison et al., (1990) J. Biol. Chem 265:20106-20116. The 3T3-L1 adipocytes were transfected with siRNA duplexes by electroporation. In brief, the adipocytes at day 5 of differentiation were detached from culture dishes with 0.25% trypsin and 0.5 mg of collagenase/ml in PBS, washed twice, and resuspended in PBS. Approximately 5 million cells (half of the cells from one p150 dish) were then mixed with siRNA duplexes, which were delivered to the cells by a pulse of electroporation with a Bio-Rad gene pulser II system (Bio-Rad Laboratories, Hercules, Calif.) at the setting of 0.18 kV and 960 μF capacitance. After electroporation, cells were immediately mixed with fresh medium for 10 min before reseeding onto multiple-well plates designed for the deoxyglucose uptake assay, Western blotting, and immunofluorescence microscopic analysis.

Immunofluorescence Microscopy. To visualize lamin A/C, cells were fixed with 4% formaldehyde and permeabilized with PBS containing 1% FBS and 0.5% Triton X-100. Cells were then incubated with primary mouse anti-rat lamin A/C Ab for overnight at 4° C. After washing, cells were incubated with FITC-labeled goat anti-mouse IgG for 30 min at room temperature. To analyze GLUT4 translocation in adipocytes, cells were cotransfected with Myc-GLUT4-EGFP plasmid and siRNAs by electroporation. After this, adipocytes were serum-starved, treated as noted in the figure legends, washed, and immunostained by using the procedure as described in Jiang et al. (2002), J. Biol. Chem. 277:509-515. In brief, the cell surface Myc-GLUT4-EGFP was visualized with mouse anti-Myc Ab and rhodamine-labeled anti-mouse secondary Ab. After washing, the coverslips were mounted in 90% glycerol containing 2.5% diazabicyclo[2.2.2]octane. Fluorescence microscopy was carried out with a IX70-inverted microscope (Olympus, Melville, N.Y.) with charge-coupled device camera (Roper Scientific, Trenton, N.J.) and METAMORPH image processing software (Universal Imaging, Downington, Pa.).

Western Blotting. After experimental treatments, the cells were solubilized as described in Jiang et al. (2002), J. Biol. Chem. 277:509-515. To detect phosphorylation of Akt Thr-308/309, GSK-3α/β Ser-21/9, and tyrosine phosphorylation of Erk-1/2, 50 μg protein from 3T3-L1 adipocyte lysates were resolved with 8% SDS/PAGE and electrotransferred to nitrocellulose membranes, which were incubated with anti-phospho-specific Abs (1:1,000 dilution) overnight at 4° C. and then with horseradish peroxidase-linked anti-rabbit IgG Abs (1:10,000 dilution) for 1 h at room temperature. Tyrosin phosphorylation of insulin receptor substrate (IRS) proteins was detected with monoclonal phosphotyrosine Ab followed by horseradish peroxidase-linked anti-mouse IgG Abs. Akt1 was detected with primary goat polyclonal Ab (1:750 dilution) and secondary horseradish peroxidase-linked donkey anti-goat Ab (1:10,000 dilution). Primary rabbit polyclonal Abs against Akt2 (1:1,000 dilution), nonmuscle myosin IIB (0.1 μg/ml), Acrp30 (0.5 μg/ml), and PKCλ/ξ (1:500 dilution) were used to detect their antigens by using 25 μg of protein from total cell lysates. The membranes were washed with wash buffer (PBS, pH 7.4/0.1% Tween 20) for 1 h at room temperature after incubation with each Ab. Finally, the levels of target proteins or phosphorylated proteins were detected with an enhanced chemiluminescence kit. To use the same nitrocellulose membrane to detect several proteins and phospho-proteins, the blots were incubated with gentle shaking in stripping buffer (62.5 mM Tris-HCl, pH 6.7/100 mM 2-mercaptoethanol/2% SDS) for 30-45 min at 60° C. and washed for at least 1 h with wash buffer before reblotting with the Ab designed for the next experiment.

The 2-Deoxyglucose Uptake Assay. Insulin-stimulated glucose transport in 3T3-L1 adipocytes was estimated by measuring 2-deoxyglucose uptake. In brief, siRNA-transfected cells were reseeded on 12-well plates and cultured for 40 h and then washed twice with DMEM before incubation with DMEM containing 0.5% BSA for 4 h at 37° C. Cells were then washed twice with Krebs-Ringer's Hepes buffer (130 mM NaCl/5 mM KCl/1.3 mM CaCl₂/1.3 mM MgSO₄/25 mM Hepes, pH 7.4) and further starved for 1.5 h with Krebs-Ringer's Hepes buffer supplemented with 0.5% BSA and 2 mM sodium pyruvate. Cells were then stimulated with insulin for 30 min at 37° C. Glucose uptake was initiated by addition of [1,2-³H]2-deoxy-D-glucose to a final assay concentration of 100 μM for 5 min at 37° C. Assays were terminated by four washes with ice-cold Krebs-Ringer's Hepes buffer, and the cells were solubilized with 0.4 ml of 1% Triton X-100, and ³H was determined by scintillation counting. Nonspecific deoxyglucose uptake was measured in the presence of 20 μM cytochalasin B and subtracted from each determination to obtain specific uptake.

Results and Discussion

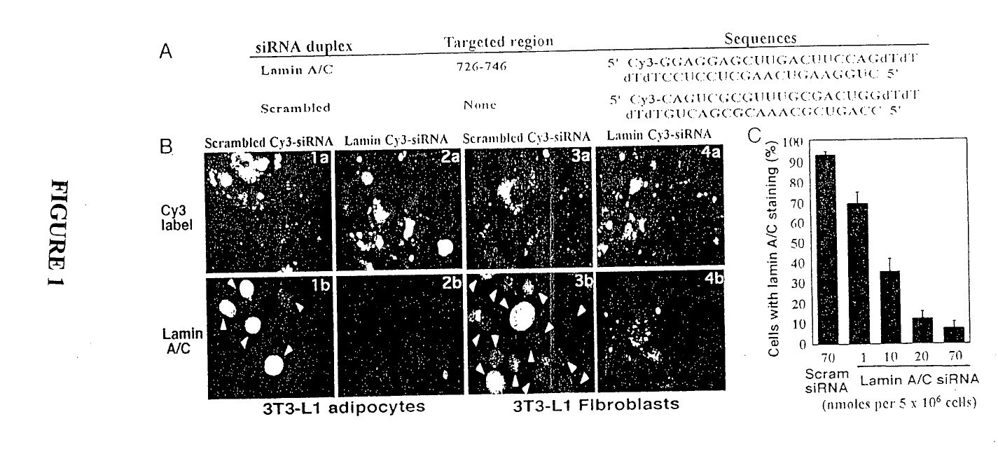

siRNA-Induced Gene Silencing in Cultured Adipocytes. To test whether siRNA can induce gene-specific silencing in adipocytes, the gene encoding lamin A/C, previously shown to be sensitive to this technique in other cells, was first targeted. Cy3-tagged 21-nt siRNA duplexes directed against mouse lamin A/C mRNA were designed and synthesized (FIG. 1A). The initial experiments showed that conditions developed for siRNA-mediated gene silencing in other cells types (Elbashir et al. (2001) Nature 411:494-498) worked well in 3T3-L1 fibroblasts (FIG. 1B Right), but failed to work in 3T3-L1 adipocytes (not shown). Consequently, an alternate methodology was developed to transfect lamin A/C siRNA into 3T3-L1 adipocytes by using electroporation. With this method, Cy3-siRNA was introduced with virtually 100% efficiency into the cultured adipocytes, and by 48 h nearly all cells showed loss of nuclear lamin A/C compared to cells transfected with a scrambled Cy3-tagged siRNA species (FIG. 1B). Quantification of these results showed that adding 20 nmol of siRNA to a suspension of 5×10⁶ adipocytes results in loss of lamin A/C in ≈90% of the adipocytes with no detectable toxicity (FIG. 1C). These findings provide the basis for reliable gene silencing in insulin-sensitive cultured adipocytes.

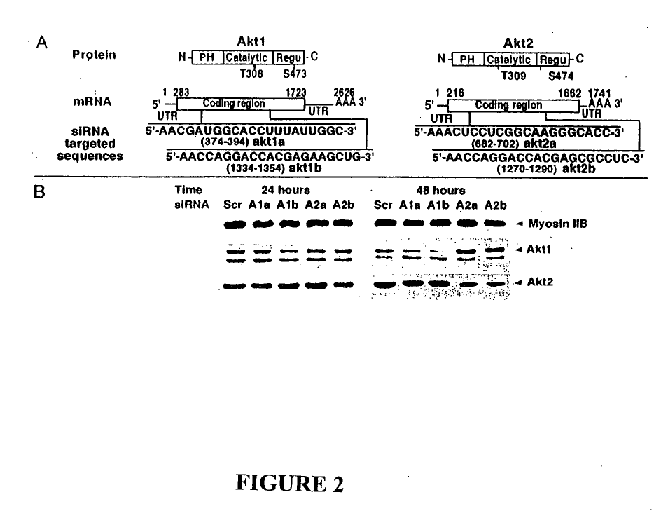

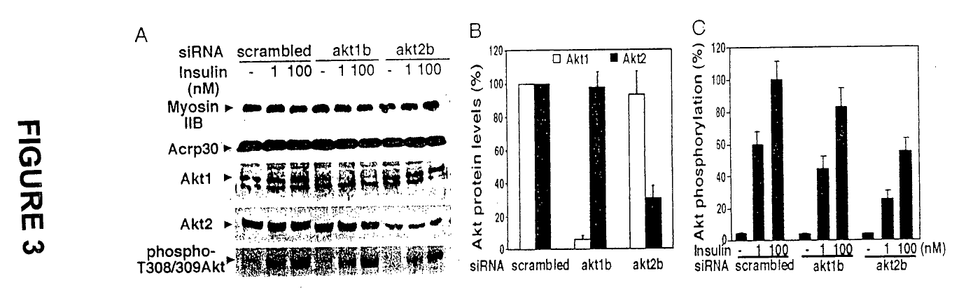

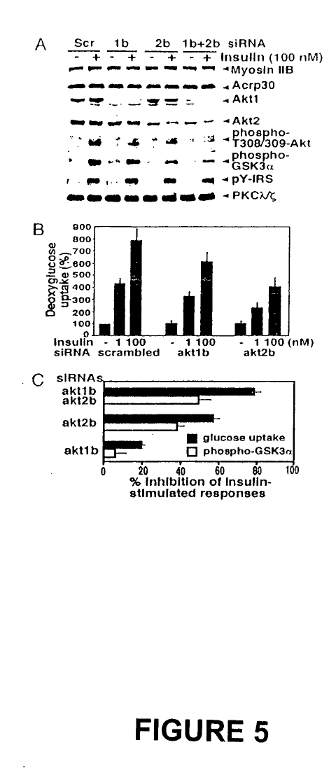

Two siRNA species directed against each of the Akt isoforms Akt1 and Akt2 were then tested for their abilities to inhibit expression of these protein kinases in 3T3-L1 adipocytes (FIG. 2). Each of the Akt1-directed siRNA species inhibited expression of Akt1 protein at both 24 and 48 h after transfection. One of these (akt1b) directed virtually total ablation of Akt1 expression by 48 h, whereas Akt2 expression was unaffected (FIGS. 2B and 3A and B). Similarly, Akt2 expression could be selectively attenuated by ≈70% after transfection of the siRNA species akt2b, whereas the akt2a siRNA was less effective (FIGS. 2B and 3A and B). Interestingly, the akt1b and akt2b siRNA species that show most efficacy are targeted to similar regions of the Akt1 and Akt2 mRNA sequences that encode amino acids 351-357 and 352-358 in Akt1 and Akt2, respectively (FIG. 2A). Whether the secondary structure of this Akt mRNA region is particularly susceptible to siRNA-directed RNA degradation requires further study. The selectivity of akt1b vs. akt2b siRNAs to silence their respective target mRNA species is apparent even though only 4 of 21 nucleotides are different (FIG. 2A). Expression of several other unrelated proteins (e.g., myosin IIB shown in FIGS. 2, 3 and 5 and adipocyte-specific protein Acrp30 shown in FIGS. 3 and 5) were also unaffected by akt1b or akt2b siRNAs.

Differential Effects of Akt1 and Akt2 Gene Silencing on Insulin Signaling. Akt1 and Akt2 are phosphorylated and activated by the protein kinase PDK1 at Thr-308 or Thr-309, respectively, in the activation T-loop, and further activation occurs through phosphorylatian at Ser-473 or Ser-474, respectively (Alessi et al. (1997) Curr. Biol. 7:776-789; Williams et al. (2000) Curr. Biol. 10:439-448; Brazil et al. (2001) Trends Biochem. Sci. 26:657-664). The effects of selective loss of Akt1 vs. Akt2 proteins on insulin-stimulated phosphorylation of total Thr-308/309 contained in both proteins were assessed by Western blotting with an anti-phospho-Thr-308/309 Ab. Total loss of Akt1 protein resulted in only a 10-20% reduction in total Thr-308/309 phosphorylation of Akt protein kinases in cultured 3T3-L1 adipocytes, consistent with previous results showing Akt1 is much less abundant than Akt2 in adipocytes (Hill et al. (1999) Mol. Cell. Biol. 19:7771-7781; Summers et al. (1999) J. Biol. Chem. 274: 23858-23867; Calera et al. (1998) J. Biol. Chem. 273:7201-7204). In contrast, reduction of Akt2 expression by ≈70% caused a marked 55-60% decrease in insulin-stimulated Thr phosphorylation of the Akt protein kinases (FIG. 3). Taken together, these data confirm the predominance of Akt2 over Akt1 in insulin-sensitive cultured adipocytes (Hill et al. (1999) Mol. Cell. Biol. 19:7771-7781).

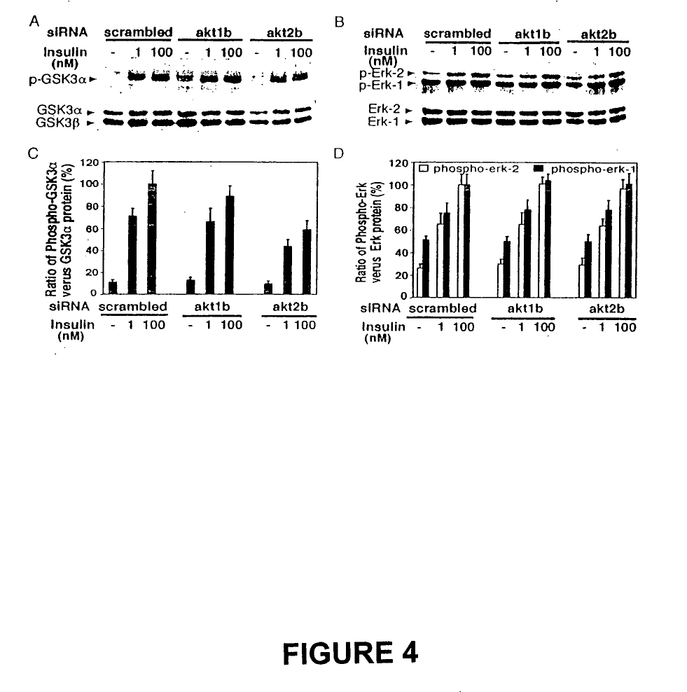

Next, the consequences of the selective attenuation of Akt1 or Akt2 expression on a downstream target of insulin signaling-GSK-3 were assessed (Cross et al. (1995) Nature 378:785-789; Cohen et al. (2001) Nat. Rev. Mol. Cell Biol. 2:769-776). GSK-3α is phosphorylated by Akt protein kinases in response to the hormone in a dose-dependent manner (FIG. 4). In three independent experiments, loss of 95% or more of Akt1 directed by the akt1b siRNA caused no significant attenuation of insulin-mediated GSK-3α phosphorylation (FIGS. 4A and B), although a 10-20% effect may go undetected in these studies. In contrast, attenuation of Akt2 expression by 70% caused ≈40% inhibition of insulin-mediated GSK-3α phosphorylation (FIGS. 4A and B and 5A and C). In control studies, no diminution of insulin signaling to the mitogen-activated protein kinases Erk-1 and Erk-2 was observed when either Akt1 or Akt2 are depleted (FIGS. 4C and D), confirming the specificity of the effect of silencing the Akt protein kinases by this method. These data indicate that insulin action on GSR-3α in cultured adipocytes specifically requires Akt2.

Redundancy of Akt1 and Akt2 in Insulin Signaling. Applying this same approach to hexose transport regulation, ablation of Akt1 expression (FIG. 5A) leads to a small but significant 20-30% decrease in insulin-stimulated 2-deoxyglucose uptake in 3T3-L1 adipocytes (FIG. 5A). Akt2 protein depletion to ≈70% of normal levels dampened the insulin response by 50-58%. These data indicate that both Akt1 and Akt2 contribute to insulin responsiveness of hexose transport in cultured adipocytes roughly in proportion to their contributions to total activated Akt in these cells (see Hill et al. (1999) Mol. Cell. Biol. 19: 7771-7781 and FIG. 3). Subsequently, the effects of depleting both Akt1 and Akt2 in 3T3-L1 adipocytes were tested by using the combination of akt1b and akt2b siRNA species (FIGS. 5A and C). This combined treatment virtually completely ablated Akt1 expression and reduced Akt2 expression by ≈65% whereas insulin-stimulated phosphorylation of total Akt detected by anti-phospho Thr-308/309 Ab decreased by 81% (FIG. 5A). Importantly, insulin-stimulated deoxyglucose uptake was inhibited by nearly 80% under these conditions, compared to ≈58% when only Akt2 was depleted (FIG. 5C). Under these conditions, GLUT4 expression was unchanged (not shown). In control experiments to test whether engagement of the gene silencing process itself nonspecifically inhibits insulin signaling, no significant effect of lamin siRNA on insulin-stimulated glucose transport were observed under conditions where lamin A/C protein levels were markedly reduced (FIG. 1 and data not shown). Taken together, these data demonstrate that although Akt2 is the major protein kinase required in this response, Akt1 can also play a role. Thus under conditions where insulin-stimulated glucose uptake is significantly compromised by partial depletion of Akt2, Akt1 is required for half of the remaining insulin signal (FIG. 5C). GSK-3α phosphorylation in response to insulin was also inhibited to a greater extent when both protein kinases were depleted versus when only Akt2 was reduced (FIGS. 5A and C).

In additional control experiments, we tested whether expression of other insulin-signaling elements such as IRS proteins (Li et al. (1999) J. Biol. Chem. 274:9351-9356) or PKCλ/ξ (Kotani et al. (1998) Mol. Cell. Biol. 18:6971-6982; Standaert et al. (1997) J. Biol. Chem. 272:30075-30082) is affected by Akt1 or Akt2 siRNAs. As shown in FIG. 5A, depletion of Akt1 or Akt2 had no significant effect on insulin-stimulated tyrosine phosphorylation of IRS proteins or expression levels of PKCλ/ξ.

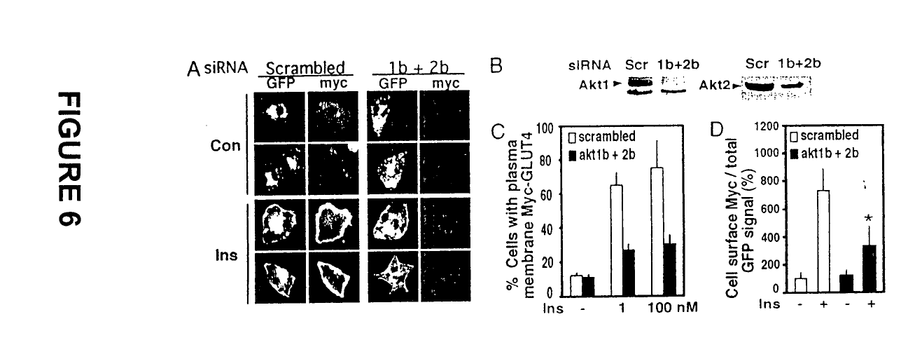

The effect of combined depletion of both Akt1 and Akt2 by siRNA on insulin-mediated GLUT4 translocation was next examined in 3T3-L1 adipocytes. Cotransfection of myc-GLUT4-EGFP plasmid DNA with the mixture of akt1b and akt2b siRNAs was performed, and 48 h later reductions of ≈90% and 65% of Akt1 and Akt2 protein levels, respectively, were observed (FIG. 6B). This combined knockdown of Akt1 and Akt2 proteins resulted in the loss of insulin-stimulated cell surface Myc signal (detected by anti-Myc Ab) in ≈70% of adipocytes transfected with the Myc-GLUT4-GFP construct (FIGS. 6A and C). Quantifying the ratio of cell surface Myc rim signal over the total Myc-GLUT4-GFP signal in positive transfected cells revealed that loss of both Akt1 and Akt2 resulted in a 60% decrease in insulin-stimulated Myc-GLUT4-GFP on the cell surface (FIG. 6D). The attenuation of GLUT4 responsiveness is observed at both maximal and submaximal concentrations of insulin (FIG. 6C), whereas no significant effect of Akt depletion on GLUT4 translocation in the absence of insulin is detected.

The findings presented here show an absolute requirement of Akt protein kinases for normal insulin signaling to glucose transport and GSK-3α and imply direct proportionality of total available Akt1 plus Akt2 to the degree of insulin responsiveness (FIG. 5). This conclusion is in keeping with the similar insulin dose-response relationships observed for activation of total Akt and glucose transport (compare FIGS. 3C and 5B). Progressive loss of Akt1, Akt2, or both leads to a correspondingly progressive loss in glucose transport stimulation (FIG. 5). These considerations show that Akt1 can in part replace Akt2 to sustain glucose transport responsiveness, potentially providing an explanation for why skeletal muscle of Akt2^{-/-} mice exhibits only a mild impairment in insulin responsiveness (Cho et al. (2001) Science 292:1728-1731). No data on insulin signaling in muscle or fat cells from Akt1^{-/-} mice have been published, but the normal glucose tolerance in these animals (Cho et al. (2001) J. Biol. Chem. 276:38349-38352) is consistent with our data (FIG. 5) that Akt2 alone can sustain most of the insulin-regulated glucose uptake. Although it is possible that other signaling pathways and protein kinases are also involved in insulin action on glucose transport, the results underscore the absolute requirement of Akt for this process.

### EXAMPLE 2

### Identification And Characterization of Akt Substrates

The present invention relates to the fields of diabetes and insulin resistance. Insulin response modulating compounds (e.g., insulin response stimulatory compounds) will permit restoration of insulin sensitivity and result in lower blood glucose levels. Insulin resistance results from the inability of normally insulin-responsive tissues to respond to the hormone. In normally functioning muscle and fat cells, insulin binds to its receptor on the surface of the cell and initiates a series of intracellular events including the transport of glucose into the cell. This glucose transport is the key event that regulates the level of glucose in the blood and maintains normoglycemia. The inability to take up glucose into these cells is a condition called insulin resistance and is often found in the diabetic or pre-diabetic state. The activity and regulation of the molecules regulating glucose transport in the cell have been widely studied in the hopes that understanding their function may lead to the ability to alter that function and restore responsiveness to insulin. The insulin-responsive glucose transporter, GLUT4, is found in intracellular vesicles that are located in an insulin-sensitive intracellular compartment. In the absence of insulin, these GLUT4-containing vesicles (G4Vs) are retained in the cytosol of the cell. Upon insulin binding to its receptor at the surface of the cell the G4Vs move from this compartment to the cell surface where GLUT4 is then at the cell surface and can transport glucose into the cell. One protein that has been shown to be involved in such trafficking and the associated insulin signaling pathway is Akt. To further identify substrates involved in the trafficking and regulation of GLUT4 and, more generally, the insulin mediated response of a cell, a biochemical screen was set up to identify proteins from insulin responsive cells that interact with Akt.

Anti-Akt substrate antibody was generated, expressed in and purified from E. coli and used as an affinity reagent to bind proteins that interact with Akt. The antibody was specific for phosphorylated sites on Akt substrates. The antibody was exposed to a adipocyte cell extract. The tagged phosphorylated proteins were immunoprecipitated by techniques well known in the art. The resulting precipitated proteins were characterized through mass spectrometry. Table 1 identifies the proteins identified according to the described methodology.

Equivalents

Those skilled in the art will recognize, or be able to ascertain using no more than routine experimentation, many equivalents to the specific embodiments of the invention described herein. Such equivalents are intended to be encompassed by the following claims.

1. A method for identifying an insulin response modulator, comprising contacting a composition comprising Akt or a bioactive fragment thereof and an Akt substrate or a bioactive fragment thereof with a test compound and determining the ability of the test compound to modulate an activity selected from the group consisting of:
(a) an interaction of Akt or the Akt bioactive fragment to the substrate or the substrate bioactive fragment;
(b) an activity of Akt or the Akt bioactive fragment;
(c) an activity of the substrate or the substrate bioactive fragment; and
(d) the phosphorylation state of the Akt substrate or the substrate bioactive fragment;
such that the insulin response modulator is identified.

2-4. (canceled)

5. The method of claim 1, wherein the interaction of Akt or the Akt bioactive fragment to the substrate or the substrate bioactive fragment comprises binding of Akt or the Akt bioactive fragment to the substrate or the substrate bioactive fragment.

6. The method of claim 1, wherein the Akt substrate is selected from the group consisting of R-type calcium channel alpha-1E subunit (R-CaC1E), WNK1, FMS interacting protein (FMIP), nGAP-like protein, nuclear matrix protein p84, HIRA interacting protein 3 (HIRIP3), HSP71, ribosomal protein L6, guanine nucleotide exchange factor Lbc (GEF Lbc), ATP citrate lyase, Mi-2b, peripheral benzodiazepine receptor-associated protein 1, heterogeneous nuclear ribonucleoprotein U (hnRNP U protein), pyruvate carboxylase precursor, Eps domain containing protein (RalBP1), nonmuscle myosin IIA (NMMIIA), and stress 70 protein (p66 mot1/GRP75).

7. The method of any one of claim 1, wherein the Akt comprises Akt1 or Akt2.

8. The method of claim 1, wherein at least one of Akt, the Akt bioactive fragment, the Akt substrate or the substrate bioactive fragment is detectably labeled.

9. The method of claim 1, wherein at least one of Akt, the Akt bioactive fragment, the Akt substrate or the substrate bioactive fragment is radioactively labeled.

10. The method of claim 1, wherein at least one of Akt, the Akt bioactive fragment, the Akt substrate or the substrate bioactive fragment is fluorescently labeled.

11. The method of claim 1, wherein the interaction is compared to an appropriate control.

12. The method of claim 1, wherein the activity is compared to an appropriate control.

13. The method of claim 1, wherein the phosphorylation state is compared to an appropriate control.

14. The method of claim 1, wherein at least one of Akt, the Akt bioactive fragment, the Akt substrate or the substrate bioactive fragment is immobilized.

15. The method of claim 1, wherein the activity of Akt or the bioactive fragment thereof is selected from the group consisting of regulation of insulin signaling to glycogen synthase kinase, regulation of intracellular GLUT4 trafficking and regulation of intracellular retention of GLUT4.

16. The method of claim 1, wherein the activity of the Akt substrate or substrate bioactive fragment is an activity set forth in Table 1 or in subsections IA-IQ.

17. The method of claim 1, wherein the Akt substrate is R-type calcium channel alpha 1E subunit.

18. The method of claim 1, wherein the Akt substrate is WNK1.

19. The method of claim 1, wherein the Akt substrate is ribosomal protein L6, and the activity comprises involvement in phosphorylation.

20. The method of claim 1, wherein the Akt substrate is guanine nucleotide exchange factor Lbc (GEF Lbc).

21. The method of claim 1, wherein the Akt substrate is ATP citrate lyase.

22. The method of claim 1, wherein the Akt bioactive fragment comprises an Akt substrate interacting portion of Akt.

23. A method for identifying an insulin response modulator, comprising contacting a cell that expresses an Akt substrate or a bioactive fragment thereof and Akt or a bioactive fragment thereof with a test compound and determining the ability of the test compound to modulate an activity selected from the group consisting of:
(a) an interaction of the Akt substrate or the substrate bioactive fragment to Akt or the Akt bioactive fragment;
(b) an activity of the Akt or the Akt bioactive fragment;
(c) an activity of the Akt substrate or the substrate bioactive fragment: and
(d) the phosphorylation state of the Akt substrate or the substrate bioactive fragment;
such that the insulin response modulator is identified.

24-26. (canceled)

27. The method of claim 23, wherein the interaction of Akt or the Akt bioactive fragment to the substrate or the substrate bioactive fragment comprises binding of Akt or the Akt bioactive fragment to the substrate or the substrate bioactive fragment.

28. The method of claim 23, wherein the Akt substrate is selected from the group consisting of R-type calcium channel alpha-1E subunit (R-CaC1E), WNK1, FMS interacting protein (FMIP), nGAP-like protein, nuclear matrix protein p84, HIRA interacting protein 3 (HIRIP3), HSP71, ribosomal protein L6, guanine nucleotide exchange factor Lbc (GEF Lbc), ATP citrate lyase, Mi-2b, peripheral benzodiazepine receptor-associated protein 1, heterogeneous nuclear ribonucleoprotein U (hnRNP U protein), pyruvate carboxylase precursor, Eps domain containing protein (RalBP1), nonmuscle myosin IIA (NMMIIA), and stress 70 protein (p66 mot1/GRP75).

29. The method of claim 23, wherein the activity of Akt or the bioactive fragment thereof is selected from the group consisting of regulation of insulin signaling to glycogen synthase kinase, regulation of intracellular GLUT4 trafficking and regulation of intracellular retention of GLUT4.

30. The method of claim 23, wherein the activity of the substrate is an activity set forth in Table 1 or in subsections IA-IQ.

31. The method of claim 23, wherein the Akt comprises Akt1 or Akt2.

32. The method of claim 23, wherein the Akt substrate is R-type calcium channel alpha 1E subunit.

33. The method of claim 23, wherein the Akt substrate is WNK1.

34. The method of claim 23, wherein the Akt substrate is ribosomal protein L6, and the activity comprises involvement in phosphorylation.

35. The method of claim 23, wherein the Akt substrate is guanine nucleotide exchange factor Lbc (GEF Lbc).

36. The method of claim 23, wherein the Akt substrate is ATP citrate lyase.

37. The method of claim 23, wherein the Akt bioactive fragment comprises an Akt substrate interacting portion of Akt.

38. The method of claim 23, wherein said cell overexpresses the Akt substrate or the bioactive fragment thereof.

39. The method of claim 23, wherein said cell overexpresses Akt or the bioactive fragment thereof.

40. The method of claim 23, wherein said cell overexpresses the Akt substrate or the substrate bioactive fragment and Akt or the Akt bioactive fragment.

41. The method of claim 1 or 23, wherein the modulator identified is a positive modulator.

42. The method of claim 1 or 23, wherein the modulator identified is a negative modulator.

43. A modulator identified by claim 1 or 23.

44. A method for identifying an Akt:Akt substrate modulator, comprising contacting a cell or a composition comprising Akt or a bioactive fragment thereof and an Akt substrate or a bioactive fragment thereof with a test compound and determining the ability of the test compound to affect an activity selected from the group consisting of:
(a) an interaction of the Akt or the bioactive fragment thereof to the Akt substrate or the bioactive fragment thereof;
(b) an activity of the Akt or the bioactive fragment thereof;
(c) an activity of the substrate or the bioactive fragment thereof; and
(d) the phosphorylation state of the Akt substrate or the bioactive fragment thereof;
such that the modulator is identified.

45-47. (canceled)

48. The method of claim 44 wherein the interaction of the Akt or the bioactive fragment thereof to the substrate or the bioactive fragment thereof is the binding of the Akt or the bioactive fragment thereof to the substrate or the bioactive fragment thereof.

49. The method of claim 44, wherein the ability of the test compound to affect comprises the ability of the test compound to either enhance or inhibit.

50. The method claim 44, wherein the Akt substrate is selected from the group consisting of R-type calcium channel alpha-1E subunit (R-CaC1E), WNK1, FMS interacting protein (FMIP), nGAP-like protein, nuclear matrix protein p84, HIRA interacting protein 3 (HIRIP3), HSP71, ribosomal protein L6, guanine nucleotide exchange factor Lbc (GEF Lbc), ATP citrate lyase, Mi-2b, peripheral benzodiazepine receptor-associated protein 1, heterogeneous nuclear ribonucleoprotein U (hnRNP U protein), pyruvate carboxylase precursor, Eps domain containing protein (RalBP1), nonmuscle myosin IIA (NMMIIA), and stress 70 protein (p66 mot1/GRP75).

51. The method of any one of claim 44, wherein the Akt comprises Akt1 or Akt2.

52. A method of modulating insulin responsiveness in a subject comprising administering to the subject an insulin response modulator identified according to the methods of any one of claims 1, 23 or 44 such that insulin responsiveness is modulated.

53. A method of regulating glucose transport in a subject comprising administering to the subject an insulin response modulator identified according to the methods of any one of claims 1, 23 or 44 such that glucose transport is regulated.

54. A method of regulating gluconeogenesis in a subject comprising administering to the subject an insulin response modulator identified according to the methods of any one of claims 1, 23 or 44 such that gluconeogenesis is regulated.

55. A method of regulating glucose homeostasis in a subject comprising administering to the subject an insulin response modulator identified according to the methods of any one of claims 1, 23 or 44 such that glucose homeostasis is regulated.

56. A method of regulating blood glucose levels in a subject comprising administering to the subject an insulin response modulator identified according to the methods of any one of claims 1, 23 or 44, such that blood glucose levels are regulated.

57. An antibody that specifically binds to an Akt-interacting domain of an Akt substrate, said antibody being capable of interfering with the Akt:Akt substrate interaction.

58. The method of claim 57, wherein the Akt substrate is selected from the group consisting of R-type calcium channel alpha-1E subunit (R-CaC1E), WNK1, FMS interacting protein (FMIP), nGAP-like protein, nuclear matrix protein p84, HIRA interacting protein 3 (HIRIP3), HSP71, ribosomal protein L6, guanine nucleotide exchange factor Lbc (GEF Lbc), ATP citrate lyase, Mi-2b, peripheral benzodiazepine receptor-associated protein 1, heterogeneous nuclear ribonucleoprotein U (hnRNP U protein), pyruvate carboxylase precursor, Eps domain containing protein (RalBP1), nonmuscle myosin IIA (NMMIIA), and stress 70 protein (p66 mot1/GRP75).

59. A pharmaceutical composition comprising the antibody of claim 57.

60. A pharmaceutical composition comprising the modulator of claim 43.

61. A pharmaceutical composition comprising an Akt-interacting domain of an Akt substrate, said Akt-interacting domain being capable of interfering with the Akt:Akt substrate interaction.

62. The composition of claim 61, wherein the Akt substrate is selected from the group consisting of R-type calcium channel alpha-1E subunit (R-CaC1E), WNK1, FMS interacting protein (FMIP), nGAP-like protein, nuclear matrix protein p84, HIRA interacting protein 3 (HIRIP3), HSP71, ribosomal protein L6, guanine nucleotide exchange factor Lbc (GEF Lbc), ATP citrate lyase, Mi-2b, peripheral benzodiazepine receptor-associated protein 1, heterogeneous nuclear ribonucleoprotein U (hnRNP U protein), pyruvate carboxylase precursor, Eps domain containing protein (RalBP1), nonmuscle myosin IIA (NMMIIA), and stress 70 protein (p66 mot1/GRP75).

63. A method of treating an insulin response disease or disorder comprising administering the pharmaceutical composition of claim 59 or 61.

64. The method of claim 63, wherein the disease or disorder is selected from the group consisting of Type I diabetes, Type II diabetes, and insulin resistance.

65. A method of treating an insulin response disease or disorder comprising administering the pharmaceutical composition of claim 60.

66. The method of claim 65, wherein the disease or disorder is selected from the group consisting of Type I diabetes, Type II diabetes, and insulin resistance.


SEQUENCE LISTING (31 sequences)

SEQ ID NO: 1 - DNA, 21 bp, Artificial Sequence
  Description of Combined DNA/RNA Molecule Synthetic siRNA oligonucleotide
ggaggagcuugacuuccagtt

SEQ ID NO: 2 - DNA, 21 bp, Artificial Sequence
  Description of Combined DNA/RNA Molecule Synthetic siRNA oligonucleotide
cagucgcguuugcgacuggtt

SEQ ID NO: 3 - DNA, 21 bp, Artificial Sequence
  Description of Combined DNA/RNA Molecule Synthetic siRNA oligonucleotide
aacgauggcaccuuuauuggc

SEQ ID NO: 4 - DNA, 21 bp, Artificial Sequence
  Description of Combined DNA/RNA Molecule Synthetic siRNA oligonucleotide
aaccaggaccacgagaagcug

SEQ ID NO: 5 - DNA, 21 bp, Artificial Sequence
  Description of Combined DNA/RNA Molecule Synthetic siRNA oligonucleotide
aaacuccucggcaagggcacc

SEQ ID NO: 6 - DNA, 21 bp, Artificial Sequence
  Description of Combined DNA/RNA Molecule Synthetic siRNA oligonucleotide
aaccaggaccacgagcgccuc

SEQ ID NO: 7 - protein, 2251 aa, Homo sapiens
MARFGEAVVARPGSGDGDSDQSRNRQGTPVPASGQAAAYKQTKAQRARTMALYNPIPVRQNCFTVNRSLFIFGEDNIVRKYAKKLIDWPPFEYMILATIIANCIVLALEQHLPEDDKTPMSRRLEKTEPYFIGIFCFEAGIKIVALGFIFHKGSYLRNGWNVMDFIVVLSGILATAGTHFNTHVDLRTLRAVRVLRPLKLVSGIPSLQIVLKSIMKAMVPLLQIGLLLFFAILMFAIIGLEFYSGKLHRACFMNNSGILEGFDPPHPCGVQGCPAGYECKDWIGPNDGITQFDNILFAVLTVFQCITMEGWTTVLYNTNDALGATWNWLYFIPLIIIGSFFVLNLVLGVLSGEFAKERERVENRRAFMKLRRQQQIERELNGYRAWIDKAEEVMLAEENKNAGTSALEVLRRATIKRSRTEAMTRDSSDEHCVDISSVGTPLARASIKSAKVDGVSYFRHKERLLRISIRHMVKSQVFYWIVLSLVALNTACVAIVHHNQPQWLTHLLYYAEFLFLGLFLLEMSLKMYGMGPRLYFHSSFNCFDFGVTVGSIFEVVWAIFRPGTSFGISVLRALRLLRIFKITKYWASLRNLVVSLMSSMKSIISLLFLLFLFIVVFALLGMQLFGGRFNFNDGTPSANFDTFPAAIMTVFQILTGEDWNEVMYNGIRSQGGVSSGMWSAIYFIVLTLFGNYTLLNVFLAIAVDNLANAQELTKDEQEEEEAFNQKHALQKAKEVSPMSAPNMPSIERERRRRHHMSVWEQRTSQLRKHMQMSSQEALNREEAPTMNPLNPLNPLSSLNPLNAHPSLYRRPRAIEGLALGLALEKFEEERISRGGSLKGDGGDRSSALDNQRTPLSLGQREPPWLARPCHGNCDPTQQEAGGGEAVVTFEDRARHRQSQRRSRHRRVRTEGKESSSASRSRSASQERSLDEAMPTEGEKDHELRGNHGAKEPTIQEERAQDLRRTNSLMVSRGSGLAGGLDEADTPLVLPHPELEVGKHVVLTEQEPEGSSEQALLGNVQLDMGRVISQSEPDLSCITANTDKATTESTSVTVAIPDVDPLVDSTVVHISNKTDGEASPLKEAEIREDEEEVEKKKQKKEKRETGKAMVPHSSMFIFSTTNPIRRACHYIVNLRYFEMCILLVIAASSIALAAEDPVLTNSERNKVLRYFDYVFTGVFTFEMVIKMIDQGLILQDGSYFRDLWNILDFVVVVGALVAFALANALGTNKGRDIKTIKSLRVLRVLRPLKTIKRLPKLKAVFDCVVTSLKNVFNILIVYKLFMFIFAVIAVQLFKGKFFYCTDSSKDTEKECIGNYVDHEKNKMEVKGREWKRHEFHYDNIIWALLTLFTVSTGEGWPQVLQHSVDVTEEDRGPSRSNRMEMSIFYVVYFVVFPFFFVNIFVALIIITFQEQGDKMMEECSLEKNERACIDFAISAKPLTRYMPQNRHTFQYRVWHFVVSPSFEYTIMAMIALNTVVLMMKYYSAPCTYELALKYLNIAFTMVFSLECVLKVIAFGFLNYFRDTWNIFDFITVIGSITEIILTDSKLVNTSGFNMSFLKLFRAARLIKLLRQGYTIRILLWTFVQSFKALPYVCLLIAMLFFIYAIIGMQVFGNIKLDEESHINRHNNFRSFFGSLMLLFRSATGEAWQEIMLSCLGEKGCEPDTTAPSGQNENERCGTDLAYVYFVSFIFFCSFLMLNLFVAVIMDNFEYLTRDSSILGPHHLDEFVRVWAEYDRAACGRIHYTEMYEMLTLMSPPLGLGKRCPSKVAYKRLVLMNMPVAEDMTVHFTSTLMALIRTALDIKIAKGGADRQQLDSELQKETLAIWPHLSQKMLDLLVPMPKASDLTVGKIYAAMMIMDYYKQSKVKKQRQQLEEQKNAPMFQRMEPSSLPQEIIANAKALPYLQQDPVSGLSGRSGYPSMSPLSPQDIFQLACMDPADDGQFQERQSLVVTDPSSMRRSFSTIRDKRSNSSWLEEFSMERSSENTYKSRRRSYHSSLRLSAHRLNSDSGHKSDTHPSGGRERRRSKERKHLLSPDVSRCNSEERGTQADWESPERRQSRSPSEGRSQTPNRQGTGSLSESSIPSVSDTSTPRRSRRQLPPVPPKPRPLLSYSSLIRHAGSISPPADGSEEGSPLTSQALESNNAWLTESSNSPHPQQRQHASPQRYISEPYLALHEDSHASDCVEEETLTFEAAVATSLGRSNTIGSAPPLRHSWQMPNGHYRRRRRGGPGPGMMCGAVNNLLSDTEEDDKC

SEQ ID NO: 8 - protein, 2382 aa, Homo sapiens
MSGGAAEKQSSTPGSLFLSPPAPAPKNGSSSDSSVGEKLGAAAADAVTGRTEEYRRRRHTMDKDSRGAAATTTTTEHRFFRRSVICDSNATALELPGLPLSLPQPSIPAAVPQSAPPEPHREETVTATATSQVAQQPPAAAAPGEQAVAGPAPSTVPSSTSKDRPVSQPSLVGSKEEPPPARSGSGGGSAKEPQEERSQQQDDIEELETKAVGMSNDGRFLKFDIEIGRGSFKTVYKGLDTETTVEVAWCELQDRKLTKSERQRFKEEAEMLKGLQHPNIVRFYDSWESTVKGKKCIVLVTELMTSGTLKTYLKRFKVMKIKVLRSWCRQILKGLQFLHTRTPPIIHRDLKCDNIFITGPTGSVKIGDLGLATLKRASFAKSVIGTPEFMAPEMYEEKYDESVDVYAFGMCMLEMATSEYPYSECQNAAQIYRRVTSGVKPASFDKVAIPEVKEIIEGCIRQNKDERYSIKDLLNHAFFQEETGVRVELAEEDDGEKIAIKLWLRIEDIKKLKGKYKDNEAIEFSFDLERDVPEDVAQEMVESGYVCEGDHKTMAKAIKDRVSLIKRKREQRQLVREEQEKKKQEESSLKQQVEQSSASQTGIKQLPSASTGIPTASTTSASVSTQVEPEEPEADQHQQLQYQQPSISVLSDGTVDSGQGSSVFTESRVSSQQTVSYGSQHEQAHSTGTVPGHIPSTVQAQSQPHGVYPPSSVAQGQSQGQPSSSSLTGVSSSQPIQHPQQQQGIQQTAPPQQTVQYSLSQTSTSSEATTAQPVSQPQAPQVLPQVSAGKQLPVSQPVPTIQGEPQIPVATQPSVVPVHSGAHFLPVGQPLPTPLLPQYPVSQIPISTPHVSTAQTGFSSLPITMAAGITQPLLTLASSATTAAIPGVSTVVPSQLPTLLQPVTQLPSQVHPQLLQPAVQSMGIPANLGQAAEVPLSSGDVLYQGFPPRLPPQYPGDSNIAPSSNVASVCIHSTVLSPPMPTEVLATPGYFPTVVQPYVESNLLVPMGGVGGQVQVSQPGGSLAQAPTTSSQQAVLESTQGVSQVAPAEPVAVAQPQATQPTTLASSVDSAHSDVASGMSDGNENVPSSSGRHEGRTTKRHYRKSVRSRSRHEKTSRPKLRILNVSNKGDRVVECQLETHNRKMVTFKFDLDGDNPEEIATIMVNNDFILAIERESFVDQVREIIEKADEMLSEDVSVEPEGDQGLESLQGKDDYGFSGSQKLEGEFKQPIPASSMPQQIGIPTSSLTQVVHSAGRRFIVSPVPESRLRESKVFPSEITDTVAASTAQSPGMNLSHSASSLSLQQAFSELRRAQMTEGPNTAPPNFSHTGPTFPVVPPFLSSIAGVPTTAAATAPVPATSSPPNDISTSVIQSEVTVPTEEGIAGVATSTGVVTSGGLPIPPVSESPVLSSVVSSITIPAVVSISTTSPSLQVPTSTSEIVVSSTALYPSVTVSATSASAGGSTATPGPKPPAVVSQQAAGSTTVGATLTSVSTTTSFPSTASQLSIQLSSSTSTPTLAETVVVSAHSLDKTSHSSTTGLAFSLSAPSSSSSPGAGVSSYISQPGGLHPLVIPSVIASTPILPQAAGPTSTPLLPQVPSIPPLVQPVANVPAVQQTLIHSQPQPALLPNQPHTHCPEVDSDTQPKAPGIDDIKTLEEKLRSLFSEHSSSGAQHASVSLETSLVIESTVTPGIPTTAVAPSKLLTSTTSTCLPPTNLPLGTVALPVTPVVTPGQVSTPVSTTTSGVKPGTAPSKPPLTKAPVLPVGTELPAGTLPSEQLPPFPGPSLTQSQQPLEDLDAQLRRTLSPEIITVTSAVGPVSMAAPTAITEAGTQPQKGVSQVKEGPVLATSSGAGVFKMGRFQVSVAADGAQKEGKNKSEDAKSVHFESSTSESSVLSSSSPESTLVKPEPNGITIPGISSDVPESAHKTTASEAKSDTGQPTKVGRFQVTTTANKVGRFSVSKTEDKITDTKKEGPVASPPFMDLEQAVLPAVIPKKEKPELSEPSHLNGPSSDPEAAFLSRDVDDGSGSPHSPHQLSSKSLPSQNLSQSLSNSFNSSYMSSDNESDIEDEDLKLELRRLRDKHLKEIQDLQSRQKHEIESLYTKLGKVPPAVIIPPAAPLSGRRRRPTKSKGSKSSRSSSLGNKSPQLSGNLSGQSAASVLHPQQTLHPPGNIPESGQNQLLQPLKPSPSSDNLYSAFTSDGAISVPSLSAPGQGTSSTNTVGATVNSQAAQAQPPAMTSSRKGTFTDDLHKLVDNWARDAMNLSGRRGSKGHMNYEGPGMARKFSAPGQLCISMTSNLGGSAPISAASATSLGHFTKSMCPPQQYGFPATPFGAQWSGTGGPAPQPLGQFQPVGTASLQNFNISNLQKSISNPPGSNLRTT

SEQ ID NO: 9 - protein, 683 aa, Homo sapiens [sequence omitted]

SEQ ID NO: 10 - protein, 967 aa, Homo sapiens
MENLRRAVHPNKDNSRRVEHILKLWVIEAKDLPAKKKYLCELCLDDVLYARTTGKLKTDNVFWGEHFEFHNLPPLRTVTVHLYRETDKKKKKERNSYLGLVSLPAASVAGRQFVEKWYPVVTPNPKGGKGPGPMIRIKARYQTITILPMEMYKEFAEHITNHYLGLCAALEPILSAKTKEEMASALVHILQSTGKVKDFLTDLMMSEVDRCGDNEHLIFRENTLATKAIEEYLKLVGQKYLQDALGEFIKALYESDENCEVDPSKCSAADLPEHQGNLKMCCELAFCKTINSYCVFPRELKEVFASWRQECSSRGRPDISERLISASLFLRFLCPAIMSPSLFNLLQEYPDDRTARTLTLIAKVTQNLANFAKFGSKEEYMSFMNQFLEHEWTNMQRFLLEISNPETLSNTAGFEGYIDLGRELSSLHSLLWEAVSQLEQSIVSKLGPLPRILRDVHTALSTPGSGQLPGTNDLASTPGSGSSSISAGLQKMVIENDLSGLIDFTRLPSPTPENKDLFFVTRSSGVQPSPARSSSYSEANEPDLQMANGGKSLSMVDLQDARTLDGEAGSPAGPDVLPTDGQAAAAQLVAGWPARATPVNLAGLATVRRAGQTPTTPGTSEGAPGRPQLLAPLSFQNPVYQMAAGLPLSPRGLGDSGSEGHSSLSSHSNSEELAAAAKLGSFSTAAEELARRPGELARRQMSLTEKGGQPTVPRQNSAGPQRRIDQPSPPPPPPPPAPRGRTPPNLLSTLQYPRPSSGTLASASPDWVGPSTRLRQQSSSSKGDSPELKPRAVHKQGPSPVSPNALDRTAAWLLTMNAQLLEDEGLGPDPPHRDRLRSKDELSQAEKDLAVLQDKLRISTKKLEEYETLFKCQEETTQKLVLEYQARLEEGEERLRRHEEDKDIQMKGIISRLMSVEEELKKDHAEMQAAVDSKQKIIDAQEKRIASLDAANARLMSALTQLKESMH

SEQ ID NO: 11 - protein, 657 aa, Homo sapiens
MSPTPPLFSLPEARTRFTKSTREALNNKNIKPLLSTFSQVPGSENEKKCTLDQAFRGILEEEIINHSSCENVLAIISLAIGGVTEGICTASTPFVLLGDVLDCLPLDQCDTIFTFVEKNVATWKSNTFYAAGKNYLLRMCNDLLRRLSKSQNTVFCGRIQLFLARLFPLSEKSGLNLQSQFNLENVTVFNTNEQESTLGQKHTEDREEGMDVEEGEMGDEEAPTTCSIPIDYNLYRKFWSLQDYFRNPVQCYEKISWKTFLKYSEEVLAVFKSYKLDDTQASRKKMEELKTGGEHVYFAKFLTSEKLMDLQLSDSNFRRHILLQYLILFQYLKGQVKFKSSNYVLTDEQSLWIEDTTKSVYQLLSENPPDGERFSKMVEHILNTEENWNSWKNEGCPSFVKERTSDTKPTRIIRKRTAPEDFLGKGPTKKILTGNEELTRLWNLCPDNMEACKSETREHMPTLEEFFEEAIEQADPENMAENEYKAMNNSNYGWRALKLLARRSPHFFQPTNQQFKSLQEYLENMVIKLAKELPPPSEEIKTGEDEDEEDNDALLKENESPDVRRDKPVTGEQIEVFANKLGEQWKILAPYLEMKDSEIRQIECDSEDMKMRAKQLLVAWQDQEGVHATPENLINALNKSGLSDLAESLTNDNETNS

SEQ ID NO: 12 - protein, 556 aa, Homo sapiens
MAREKEMQEFTRSFFRGRPDLSTLTHSIVRRRYLAHSGRSHLEPEEKQALKRLVEEELLKMQVDEAASREDKLDLTKKGKRPPTPCSDPERKRFRFNSESESGSEASSPDYFGPPAKNGVAAEVSPAKEENPRRASKAVEESSDEERQRDLPAQRGEESSEEEEKGYKGKTRKKPVVKKQAPGKASVSRKQAREESEESEAEPVQRTAKKVEGNKGTKSLKESEQESEEEILAQKKEQREEEVEEEEKEEDEEKGDWKPRTRSNGRRKSAREERSCKQKSQAKRLLGDSDSEEEQKEAASSGDDSGRDREPPVQRKSEDRTQLKGGKRLSGSSEDEEDSGKGEPTAKGSRKMARLGSTSGEESDLEREVSDSEAGGGPQGERKNRSSKKSSRKGRTRSSSSSSDGSPEAKGGKAGSGRRGEDHPAVMRLKRYIRACGAHRNYKKLLGSCCSHKERLSILRAELEALGMKGTPSLGKCRALKEQREEAAEVASLDVANIISGSGRPRRRTAWNPLGEAAPPGELYRRTLDSDEERPRPAPPDWSHMRGIISSDGESN

SEQ ID NO: 13 - protein, 493 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGMPGGMPGGFPGGGAPPSGGASSGPTIEEVD

SEQ ID NO: 14 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 15 - protein, 288 aa, Homo sapiens [sequence omitted]

SEQ ID NO: 16 - protein, 2813 aa, Homo sapiens
MKLNPQQAPLYGDCVVTVLLAEEDKAEDDVVFYLVFLGSTLRHCTSTRKVSSDTLETIAPGHDCCETVKVQLCASKEGLPVFVVAEEDFHFVQDEAYDAAQFLATSAGNQQALNFTRFLDQSGPPSGDVNSLDKKLVLAFRHLKLPTEWNVLGTDQSLHDAGPRETLMHFAVRLGLLRLTWFLLQKPGGRGALSIHNQEGATPVSLALERGYHKLHQLLTEENAGEPDSWSSLSYEIPYGDCSVRHHRELDIYTLTSESDSHHEHPFPGDGCTGPIFKLMNIQQQLMKTNLKQMDSLMPLMMTAQDPSSAPETDGQFLPCAPEPTDPQRLSSSEETESTQCCPGSPVAQTESPCDLSSIVEEENTDRSCRKKNKGVERKGEEVEPAPIVDSGTVSDQDSCLQSLPDCGVKGTEGLSSCGNRNEETGTKSSGMPTDQESLSSGDAVLQRDLVMEPGTAQYSSGGELGGISTTNVSTPDTAGEMEHGLMNPDATVWKNVLQGGESTKERFENSNIGTAGASDVHVTSKPVDKISVPNCAPAASSLDGNKPAESSLAFSNEETSTEKTAETETSRSREESADAPVDQNSVVIPAAAKDKISDGLEPYTLLAAGIGEAMSPSDLALLGLEEDVMPHQNSETNSSHAQSQKGKSSPICSTTGDDKLCADSACQQNTVTSSGDLVAKLCDNIVSESESTTARQPSSQDPPDASHCEDPQAHTVTSDPVRDTQERADFCPFKVVDNKGQRKDVKLDKPLTNMLEVVSHPHPVVPKMEKELVPDQAVISDSTFSLANSPGSESVTKDDALSFVPSQKEKGTATPELHTATDYRDGPDGNSNEPDTRPLEDRAVGLSTSSTAAELQHGMGNTSLTGLGGEHEGPAPPAIPEALNIKGNTDSSLQSVGKATLALDSVLTEEGKLLVVSESSAAQEQDKDKAVTCSSIKENALSSGTLQEEQRTPPPGQDTQQFHEKSISADCAKDKALQLSNSPGASSAFLKAETEHNKEVAPQVSLLTQGGAAQSLVPPGASLATESRQEALGAEHNSSALLPCLLPDGSDGSDALNCSQPSPLDVGVKNTQSQGKTSACEVSGDVTVDVTGVNALQGMAEPRRENISHNTQDILIPNVLLSQEKNAVLGLPVALQDKAVTDPQGVGTPEMIPLDWEKGKLEGADHSCTMGDAEEAQIDDEAHPVLLQPVAKELPTDMELSAHDDGAPAGVREVMRAPPSGRERSTPSLPCMVSAQDAPLPKGADLIEEAASRIVDAVIEQVKAAGALLTEGEACHMSLSSPELGPLTKGLESAFTEKVSTFPPGESLPMGSTPEEATGSLAGCFAGREEPEKIILPVQGPEPAAEMPDVKAEDEVDFRASSISEEVAVGSIAATLKMKQGPMTQAINRENWCTIEPCPDAASLLASKQSPECENFLDVGLGRECTSKQGVLKRESGSDSDLFHSPSDDMDSIIFPKPEEEHLACDITGSSSSTDDTASLDRHSSHGSDVSLSQILKPNRSRDRQSLDGFYSHGMGAEGRESESEPADPGDVEEEEMDSITEVPANCSVLRSSMRSLSPFRRHSWGPGKNAASDAEMNHRSSMRVLGDVVRRPPIHRRSFSLEGLTGGAGVGNKPSSSLEVSSANAEELRHPFSGEERVDSLVSLSEEDLESDQREHRMFDQQICHRSKQQGFNYCTSAISSPLTKSISLMTISHPGLDNSRPFHSTFHNTSANLTESITEENYNFLPHSPSKKDSEWKSGTKVSRTFSYIKNKMSSSKKSKEKEKEKDKIKEKEKDSKDKEKDKKTVNGHTFSSIPVVGPISCSQCMKPFTNKDAYTCANCSAFVHKGCRESLASCAKVKMKQPKGSLQAHDTSSLPTVIMRNKPSQPKERPRSAVLLVDETATTPIFANRRSQQSVSLSKSVSIQNITGVGNDENMSNTWKFLSHSTDSLNKISKVNESTESLTDEGVGTDMNEGQLLGDFEIESKQLEAESWSRIIDSKFLKQQKKDVVKRQEVIYELMQTEFHHVRTLKIMSGVYSQGMMADLLFEQQMVEKLFPCLDELISIHSQFFQRILERKKESLVDKSEKNFLIKRIGDVLVNQFSGENAERLKKTYGKFCGQHNQSVNYFKDLYAKDKRFQAFVKKKMSSSVVRRLGIPECILLVTQRITKYPVLFQRILQCTKDNEVEQEDLAQSLSLVKDVIGAVDSKVASYEKKVRLNEIYTKTDSKSIMRMKSGQMFAKEDLKRKKLVRDGSVFLKNAAGRLKEVQAVLLTDILVFLQEKDQKYIFASLDQKSTVISLKKLIVREVAHEEKGLFLISMGMTDPEMVEVHASSKEERNSWIQIIQDTINTLNRDEDEGIPSENEEEKKMLDTRARELKEQLHQKDQKILLLLEEKEMIFRDMAECSTPLPEDCSPTHSPRVLFRSNTEEALKGGPLMKSAINEVEILQGLVSGNLGGTLGPTVSSPIEQDVVGPVSLPRRAETFGGFDSHQMNASKGGEKEEGDDGQDLRRTESDSGLKKGGNANLVFMLKRNSEQVVQSVVHLYELLSALQGVVLQQDSYIEDQKLVLSERALTRSLSRPSSLIEQEKQRSLEKQRQDLANLQKQQAQYLEEKRRREREWEARERELREREALLAQREEEVQQGQQDLEKEREELQQKKGTYQYDLERLRAAQKQLEREQEQLRREAERLSQRQTERDLCQVSHPHTKLMRIPSFFPSPEEPPSPSAPSIAKSGSLDSELSVSPKRNSISRTHKDKGPFHILSSTSQTNKGPEGQSQAPASTSASTRLFGLTKPKEKKEKKKKNKTSRSQPGDGPASEVSAEGEEIFC

SEQ ID NO: 17 - protein, 2817 aa, Homo sapiens
MKLNPQQAPLYGDCVVTVLLAEEDKAEDDVVFYLVFLGSTLRHCTSTRKVSSDTLETIAPGHDCCETVKVQLCASKEGLPVFVVAEEDFHFVQDEAYDAAQFLATSAGNQQALNFTRFLDQSGPPSGDVNSLDKKLVLAFRHLKLPTEWNVLGTDQSLHDAGPRETLMHFAVRLGLLRLTWFLLQKPGGRGALSIHNQEGATPVSLALERGYHKLHQLLTEENAGEPDSWSSLSYEIPYGDCSVRHHRELDIYTLTSESDSHHEHPFPGDGCTGPIFKLMNIQQQLMKTNLKQMDSLMPLMMTAQDPSSAPETDGQFLPCAPEPTDPQRLSSSEETESTQCCPGSPVAQTESPCDLSSIVEEENTDRSCRKKNKGVERKGEEVEPAPIVDSGTVSDQDSCLQSLPDCGVKGTEGLSSCGNRNEETGTKSSGMPTDQESLSSGDAVLQRDLVMEPGTAQYSSGGELGGISTTNVSTPDTAGEMEHGLMNPDATVWKNVLQGGESTKERFENSNIGTAGASDVHVTSKPVDKISVPNCAPAASSLDGNKPAESSLAFSNEETSTEKTAETETSRSREESADAPVDQNSVVIPAAAKDKISDGLEPYTLLAAGIGEAMSPSDLALLGLEEDVMPHQNSETNSSHAQSQKGKSSPICSTTGDDKLCADSACQQNTVTSSGDLVAKLCDNIVSESESTTARQPSSQDPPDASHCEDPQAHTVTSDPVRDTQERADFCPFKVVDNKGQRKDVKLDKPLTNMLEVVSHPHPVVPKMEKELVPDQAVISDSTFSLANSPGSESVTKDDALSFVPSQKEKGTATPELHTATDYRDGPDGNSNEPDTRPLEDRAVGLSTSSTAAELQHGMGNTSLTGLGGEHEGPAPPAIPEALNIKGNTDSSLQSVGKATLALDSVLTEEGKLLVVSESSAAQEQDKDKAVTCSSIKENALSSGTLQEEQRTPPPGQDTQQFHEKSISADCAKDKALQLSNSPGASSAFLKAETEHNKEVAPQVSLLTQGGAAQSLVPPGASLATESRQEALGAEHNSSALLPCLLPDGSDGSDALNCSQPSPLDVGVKNTQSQGKTSACEVSGDVTVDVTGVNALQGMAEPRRENISHNTQDILIPNVLLSQEKNAVLGLPVALQDKAVTDPQGVGTPEMIPLDWEKGKLEGADHSCTMGDAEEAQIDDEAHPVLLQPVAKELPTDMELSAHDDGAPAGVREVMRAPPSGRERSTPSLPCMVSAQDAPLPKGADLIEEAASRIVDAVIEQVKAAGALLTEGEACHMSLSSPELGPLTKGLESAFTEKVSTFPPGESLPMGSTPEEATGSLAGCFAGREEPEKIILPVQGPEPAAEMPDVKAEDEVDFRASSISEEVAVGSIAATLKMKQGPMTQAINRENWCTIEPCPDAASLLASKQSPECENFLDVGLGRECTSKQGVLKRESGSDSDLFHSPSDDMDSIIFPKPEEEHLACDITGSSSSTDDTASLDRHSSHGSDVSLSQILKPNRSRDRQSLDGFYSHGMGAEGRESESEPADPGDVEEEEMDSITEVPANCSVLRSSMRSLSPFRRHSWGPGKNAASDAEMNHRSMSWCPSGVQYSAGLSADFNYRSFSLEGLTGGAGVGNKPSSSLEVSSANAEELRHPFSGEERVDSLVSLSEEDLESDQREHRMFDQQICHRSKQQGFNYCTSAISSPLTKSISLMTISHPGLDNSRPFHSTFHNTSANLTESITEENYNFLPHSPSKKDSEWKSGTKVSRTFSYIKNKMSSSKKSKEKEKEKDKIKEKEKDSKDKEKDKKTVNGHTFSSIPVVGPISCSQCMKPFTNKDAYTCANCSAFVHKGCRESLASCAKVKMKQPKGSLQAHDTSSLPTVIMRNKPSQPKERPRSAVLLVDETATTPIFANRRSQQSVSLSKSVSIQNITGVGNDENMSNTWKFLSHSTDSLNKISKVNESTESLTDEGVGTDMNEGQLLGDFEIESKQLEAESWSRIIDSKFLKQQKKDVVKRQEVIYELMQTEFHHVRTLKIMSGVYSQGMMADLLFEQQMVEKLFPCLDELISIHSQFFQRILERKKESLVDKSEKNFLIKRIGDVLVNQFSGENAERLKKTYGKFCGQHNQSVNYFKDLYAKDKRFQAFVKKKMSSSVVRRLGIPECILLVTQRITKYPVLFQRILQCTKDNEVEQEDLAQSLSLVKDVIGAVDSKVASYEKKVRLNEIYTKTDSKSIMRMKSGQMFAKEDLKRKKLVRDGSVFLKNAAGRLKEVQAVLLTDILVFLQEKDQKYIFASLDQKSTVISLKKLIVREVAHEEKGLFLISMGMTDPEMVEVHASSKEERNSWIQIIQDTINTLNRDEDEGIPSENEEEKKMLDTRARELKEQLHQKDQKILLLLEEKEMIFRDMAECSTPLPEDCSPTHSPRVLFRSNTEEALKGGPLMKSAINEVEILQGLVSGNLGGTLGPTVSSPIEQDVVGPVSLPRRAETFGGFDSHQMNASKGGEKEEGDDGQDLRRTESDSGLKKGGNANLVFMLKRNSEQVVQSVVHLYELLSALQGVVLQQDSYIEDQKLVLSERALTRSLSRPSSLIEQEKQRSLEKQRQDLANLQKQQAQYLEEKRRREREWEARERELREREALLAQREEEVQQGQQDLEKEREELQQKKGTYQYDLERLRAAQKQLEREQEQLRREAERLSQRQTERDLCQVSHPHTKLMRIPSFFPSPEEPPSPSAPSIAKSGSLDSELSVSPKRNSISRTHKDKGPFHILSSTSQTNKGPEGQSQAPASTSASTRLFGLTKPKEKKEKKKKNKTSRSQPGDGPASEVSAEGEEIFC

SEQ ID NO: 18 - protein, 1058 aa, Homo sapiens
MSSSKKSKEKEKEKDKIKEKEKDSKDKEKDKKTVNGHTFSSIPVVGPISCSQCMKPFTNKDAYTCANCSAFVHKGCRESLASCAKVKMKQPKGSLQAHDTSSLPTVIMRNKPSQPKERPRSAVLLVDETATTPIFANRRSQQSVSLSKSVSIQNITGVGNDENMSNTWKFLSHSTDSLNKISKVNESTESLTDEGVGTDMNEGQLLGDFEIESKQLEAESWSRIIDSKFLKQQKKDVVKRQEVIYELMQTEFHHVRTLKIMSGVYSQGMMADLLFEQQMVEKLFPCLDELISIHSQFFQRILERKKESLVDKSEKNFLIKRIGDVLVNQFSGENAERLKKTYGKFCGQHNQSVNYFKDLYAKDKRFQAFVKKKMSSSVVRRLGIPECILLVTQRITKYPVLFQRILQCTKDNEVEQEDLAQSLSLVKDVIGAVDSKVASYEKKVRLNEIYTKTDSKSIMRMKSGQMFAKEDLKRKKLVRDGSVFLKNAAGRLKEVQAVLLTDILVFLQEKDQKYIFASLDQKSTVISLKKLIVREVAHEEKGLFLISMGMTDPEMVEVHASSKEERNSWIQIIQDTINTLNRDEDEGIPSENEEEKKMLDTRARELKEQLHQKDQKILLLLEEKEMIFRDMAECSTPLPEDCSPTHSPRVLFRSNTEEALKGGPLMKSAINEVEILQGLVSGNLGGTLGPTVSSPIEQDVVGPVSLPRRAETFGGFDSHQMNASKGGEKEEGDDGQDLRRTESDSGLKKGGNANLVFMLKRNSEQVVQSVVHLYELLSALQGVVLQQDSYIEDQKLVLSERALTRSLSRPSSLIEQEKQRSLEKQRQDLANLQKQQAQYLEEKRRREREWEARERELREREALLAQREEEVQQGQQDLEKEREELQQKKGTYQYDLERLRAAQKQLEREQEQLRREAERLSQRQTERDLCQVSHPHTKLMRIPSFFPSPEEPPSPSAPSIAKSGSLDSELSVSPKRNSISRTHKDKGPFHILSSTSQTNKGPEGQSQAPASTSASTRLFGLTKPKEKKEKKKKNKTSRSQPGDGPASEVSAEGEEIFC

SEQ ID NO: 19 - protein, 1105 aa, Homo sapiens
MSAKAISEQTGKELLYKFICTTSAIQNRFKYARVTPDTDWARLLQDHPWLLSQNLVVKPDQLIKRRGKLGLVGVDLTLDGVKSWLKPRLGQEATVGKATGFLKNFLIEPFAPHSQAEEFYVCIYATREGDYVLFHHEGGVDVGDVDAKAQKLLVGVDEKLNPEDIKKHLLVHAPDDKKEILASFISGLFNFYEDLYFTYLEINPLVVTKDGVYVLDLAAKVDATADYICKVKWGDIEFPPPFGRVAYPEEAYIADLDAKSGASLKLTLLNPKGRIWTMVAGGGASVVYSDTICDLGGVNELANYGEYSGAPSEQQTYDYAKTILSLMTREKHPDGKILIIGGSIANFTNVAATFKGIVRAIRDYQGPLKEHEVTIFVRRGGPNYQEGLRVMGEVGKTTGIPIHVFGTETHMTAIVGMAWAPAIPNQPPTAAHTANFLLNAQRETSTPAPSRTASFYESMVDEVRADEVAPAKKAKPAMPQDSVPSPRSLQGKSTTLFSRHTKAIVWGMQTRAVQGMLDFDYVCSRDEPSVAAMVYPFTGDHKQKFYWGHKEILIPVFKNMADAMRKHPEVDVLINFASLRSAYDSTMETMNYAQIRTIAIIAEGIPEALTRKLIKKADQKGVTIIGPATVGGIKPGCFKIGNTGGMLDNILASKLYPQAAVAYVSRSGGMSNELNNIISRTTDGVYEGVAIGGDRYPGSTFMDHVLRYQDTPGVKMIVVLGEIGGTEEYKISRGIKEGRLTKPIVCWCIGTCATMFSSEVQFGHAGACANQASETAVAKNQALKEAGVFVPRSFDELGEIIQSVYEDLVANGVIVPAQEVPPPTVPMDYSWARELGLIRKPASFMTSICDERGQELIYAGMPITEVFKEEMGIGGALGLLWFQKRLPKYSCQFIEMCLMVTADHGPAVSGAHNTIICARTAVELVSSLTSGLLTIGDRFGGALDAAAKMFSKAFDSGIIPMEFVNKMKKEGKLIMGIGHRVKSINNPDMRVQILKDYVRQHFPATPLLDYALEVEKITTSKKPNLILNVDGLIGVAFVDMLRNCGSFTREEADEYIDIGALNGIFVLGRSMGFIGHYLDQKRLKQGLYRHPWDDISYVLPEHMSM

SEQ ID NO: 20 - protein, 1912 aa, Homo sapiens
MASGLGSPSPCSAGSEEEDMDALLNNSLPPPHPENEEDPEEDLSETETPKLKKKKKPKKPRDPKIPKSKRQKKERMLLCRQLGDSSGEGPEFVEEEEEVALRSDSEGSDYTPGKKKKKKLGPKKEKKSKSKRKEEEEEDDDDDDSKEPKSSAQLLEDWGMEDIDHVFSEEDYRTLTNYKAFSQFVRPLIAAKNPKIAVSKMMMVLGAKWREFSTNNPFKGSSGASVAAAAAAAVAVVESMVTATEVAPPPPPVEVPIRKAKTKEGKGPNARRKPKGSPRVPDAKKPKPKKVAPLKIKLGGFGSKRKRSSSEDDDLDVESDFDDASINSYSVSDGSTSRSSRSRKKLRTTKKKKKGEEEVTAVDGYETDHQDYCEVCQQGGEIILCDTCPRAYHMVCLDPDMEKAPEGKWSCPHCEKEGIQWEAKEDNSEGEEILEEVGGDLEEEDDHHMEFCRVCKDGGELLCCDTCPSSYHIHCLNPPLPEIPNGEWLCPRCTCPALKGKVQKILIWKWGQPPSPTPVPRPPDADPNTPSPKPLEGRPERQFFVKWQGMSYWHCSWVSELQLELHCQVMFRNYQRKNDMDEPPSGDFGGDEEKSRKRKNKDPKFAEMEERFYRYGIKPEWMMIHRILNHSVDKKGHVHYLIKWRDLPYDQASWESEDVEIQDYDLFKQSYWNHRELMRGEEGRPGKKLKKVKLRKLERPPETPTVDPTVKYERQPEYLDATGGTLHPYQMEGLNWLRFSWAQGTDTILADEMGLGKTVQTAVFLYSLYKEGHSKGPFLVSAPLSTIINWEREFEMWAPDMYVVTYVGDKDSRAIIRENEFSFEDNAIRGGKKASRMKKEASVKFHVLLTSYELITIDMAILGSIDWACLIVDEAHRLKNNQSKFFRVLNGYSLQHKLLLTGTPLQNNLEELFHLLNFLTPERFHNLEGFLEEFADIAKEDQIKKLHDMLGPHMLRRLKADVFKNMPSKTELIVRVELSPMQKKYYKYILTRNFEALNARGGGNQVSLLNVVMDLKKCCNHPYLFPVAAMEAPKMPNGMYDGSALIRASGKLLLLQKMLKNLKEGGHRVLIFSQMTKMLDLLEDFLEHEGYKYERIDGGITGNMRQEAIDRFNAPGAQQFCFLLSTRAGGLGINLATADTVIIYDSDWNPHNDIQAFSRAHRIGQNKKVMIYRFVTRASVEERITQVAKKKMMLTHLVVRPGLGSKTGSMSKQELDDILKFGTEELFKDEATDGGGDNKEGEDSSVIHYDDKAIERLLDRNQDETEDTELQGMNEYLSSFKVAQYVVREEEMGEEEEVEREIIKQEESVDPDYWEKLLRHHYEQQQEDLARNLGKGKRIRKQVNYNDGSQEDRDWQDDQSDNQSDYSVASEEGDEDFDERSEAPRRPSRKGLRNDKDKPLPPLLARVGGNIEVLGFNARQRKAFLNAIMRYGMPPQDAFTTQWLVRDLRGKSEKEFKAYVSLFMRHLCEPGADGAETFADGVPREGLSRQHVLTRIGVMSLIRKKVQEFEHVNGRWSMPELAEVEENKKMSQPGSPSPKTPTPSTPGDTQPNTPAPVPPAEDGIKIEENSLKEEESIEGEKEVKSTAPETAIECTQAPAPASEDEKVVVEPPEGEEKVEKAEVKERTEEPMETEPKGAADVEKVEEKSAIDLTPIVVEDKEEKKEEEEKKEVMLQNGETPKDLNDEKQKKNIKQRFMFNIADGGFTELHSLWQNEERAATVTKKTYEIWHRRHDYWLLAGIINHGYARWQDIQNDPRYAILNEPFKGEMNRGNFLEIKNKFLARRFKLLEQALVIEEQLRRAAYLNMSEDPSHPSMALNTRFAEVECLAESHQHLSKESMAGNKPANAVLHKVLKQLEELLSDMKADVTRLPATIARIPPVAVRLQMSERNILSRLANRAPEPTPQQVAQQQ

SEQ ID NO: 21 - protein, 1857 aa, Homo sapiens
MEQLTTLPRPGDPGAMEPWALPTWHSWTPGRGGEPSSAAPSIADTPPAALQLQELRSEESSKPKGDGSSRPVGGTDPEGAEACLPSLGQQASSSGPACQRPEDEEVEAFLKAKLNMSFGDRPNLELLRALGELRQRCAILKEENQMLRKSSFPETEEKVRRLKRKNAELAVIAKRLEERARKLQETNLRVVSAPLPRPGTSLELCRKALARQRARDLSETASALLAKDKQIAALQRECRELQARLTLVGKEGPQWLHVRDFDRLLRESQREVLRLQRQIALRNQRETLPLPPSWPPGPALQARAGAPAPGAPGEATPQEDADNLPVILGEPEKEQRVQQLESELSKKRKKCESLEQEARKKQRRCEELELQLRQAQNENARLVEENSRLSGRATEKEQVEWENAELRGQLLGVTQERDSALRKSQGLQSKLESLEQVLKHMREVAQRRQQLEVEHEQARLSLREKQEEVRRLQQAQAEAQREHEGAVQLLESTLDSMQARVRELEEQCRSQTEQFSLLAQELQAFRLHPGPLDLLTSALDCGSLGDCPPPPCCCSIPQPCRGSGPKDLDLPPGSPGRCTPKSSEPAPATLTGVPRRTAKKAESLSNSSHSESIHNSPKSCPTPEVDTASEVEELEADSVSLLPAAPEGSRGGARIQVFLARYSYNPFEGPNENPEAELPLTAGEYIYIYGNMDEDGFFEGELMDGRRGLVPSNFVERVSDDDLLTSLPPELADLSHSSGPELSFLSVGGGGSSSGGQSSVGRSQPRPEEEDAGDELSLSPSPEGLGEPPAVPYPRRLVVLKQLAHSVVLAWEPPPEQVELHGFHICVNGELRQALGPGAPPKAVLENLDLWAGPLHISVQALTSRGSSDPLRCCLAVGARAGVVPSQLRVHRLTATSAEITWVPGNSNLAHAIYLNGEECPPASPSTYWATFCHLRPGTPYQAQVEAQLPPQGPWEPGWERLEQRAATLQFTTLPAGPPDAPLDVQIEPGPSPGILIISWLPVTIDAAGTSNGVRVTGYAIYADGQKIMEVASPTAGSVLVELSQLQLLQVCREVVVRTMSPHGESADSIPAPITPALAPASLPARVSCPSPHPSPEARAPLASASPGPGDPSSPLQHPAPLGTQEPPGAPPASPSREMAKGSHEDPPAPCSQEEAGAAVLGTSEERTASTSTLGEKDPGPAAPSLAKQEAEWTAGEACPASSSTQGARAQQAPNTEMCQGGDPGSGLRPRAEKEDTAELGVHLVNSLVDHGRNSDLSDIQEEEEEEEEEEEEELGSRTCSFQKQVAGNSIRENGAKSQPDPFCETDSDEEILEQILELPLQQFCSKKLFSIPEEEEEEEEDEEEEKSGAGCSSRDPGPPEPALLGLGCDSGQPRRPGQCPLSPESSRAGDCLEDMPGLVGGSSRRRGGGSPEKPPSRRRPPDPREHCSRLLSNNGPQASGRLGPTRERGGLPVIEGPRTGLEASGRGRLGPSRRCSRGRALEPGLASCLSPKCLEISIEYDSEDEQEAGSGGISITSSCYPGDREAWGTATVGRPRGPPKANSGPKPYPRLPAWEKGEPERRGRSATGRAKEPLSRATETGEARGQDGSGRRGPQKRGVRVLRPSTAELVPARSPSETLAYQHLPVRIFVALFDYDPVSMSPNPDAGEEELPFREGQILKVFGDKDADGFYQGEGGGRTGYIPCNMVAEVAVDSPAGRQQLLQRGYLSPDILLEGSGNGPFVYSTAHTTGPPPKPRRSKKAESEGPAQPCPGPPKLVPSADLKAPHSMVAAFDYNPQESSPNMDVEAELPFRAGDVITVFGGMDDDGFYYGELNGQRGLVPSNFLEGPGPEAGGLDREPRTPQAESQRTRRRRVQC

SEQ ID NO: 22 - protein, 806 aa, Homo sapiens
MSSSPVNVKKLKVSELKEELKKRRLSDKGLKAELMERLQAALDDEEAGGRPAMEPGNGSLDLGGDSAGRSGAGLEQEAAAGGDEEEEEEEEEEEGISALDGDQMELGEENGAAGAADSGPMEEEEAASEDENGDDQGFQEGEDELGDEEEGAGDENGHGEQQPQPPATQQQQPQQQRGAAKEAAGKSSGPTSLFAVTVAPPGARQGQQQAGGDGKTEQKGGDKKRGVKRPREDHGRGYFEYIEENKYSRAKSPQPPVEEEDEHFDDTVVCLDTYNCDLHFKISRDRLSASSLTMESFAFLWAGGRASYGVSKGKVCFEMKVTEKIPVRHLYTKDIDIHEVRIGWSLTTSGMLLGEEEFSYGYSLKGIKTCNCETEDYGEKFDENDVITCFANFESDEVELSYAKNGQDLGVAFKISKEVLAGRPLFPHVLCHNCAVEFNFGQKEKPYFPIPEEYTFIQNVPLEDRVRGPKGPEEKKDCEVVMMIGLPGAGKTTWVTKHAAENPGKYNILGTNTIMDKMMVAGFKKQMADTGKLNTLLQRAPQCLGKFIEIAARKKRNFILDQTNVSAAAQRRKMCLFAGFQRKAVVVCPKDEDYKQRTQKKAEVEGKDLPEHAVLKMKGNFTLPEVAECFDEITYVELQKEEAQKLLEQYKEESKKALPPEKKQNTGSKKSNKNKSGKNQFNRGGGHRGRGGFNMRGGNFRGGAPGNRGGYNRRGNMPQRGGGGGGSGGIGYPYPRAPVFPGRGSYSNRGNYNRGGMPNRGNYNQNFRGRGNNRGYKNQSQGYNQWQQGQFWGQKPWSQHYHQGYY

SEQ ID NO: 23 - protein, 824 aa, Homo sapiens
MSSSPVNVKKLKVSELKEELKKRRLSDKGLKAELMERLQAALDDEEAGGRPAMEPGNGSLDLGGDSAGRSGAGLEQEAAAGGDEEEEEEEEEEEGISALDGDQMELGEENGAAGAADSGPMEEEEAASEDENGDDQGFQEGEDELGDEEEGAGDENGHGEQQPQPPATQQQQPQQQRGAAKEAAGKSSGPTSLFAVTVAPPGARQGQQQAGGKKKAEGGGGGGRPGAPAGDGKTEQKGGDKKRGVKRPREDHGRGYFEYIEENKYSRAKSPQPPVEEEDEHFDDTVVCLDTYNCDLHFKISRDRLSASSLTMESFAFLWAGGRASYGVSKGKVCFEMKVTEKIPVRHLYTKDIDIHEVRIGWSLTTSGMLLGEEEFSYGYSLKGIKTCNCETEDYGEKFDENDVITCFANFESDEVELSYAKNGQDLGVAFKISKEVLAGRPLFPHVLCHNCAVEFNFGQKEKPYFPIPEEYTFIQNVPLEDRVRGPKGPEEKKDCEVVMMIGLPGAGKTTWVTKHAAENPGKYNILGTNTIMDKMMVAGFKKQMADTGKLNTLLQRAPQCLGKFIEIAARKKRNFILDQTNVSAAAQRRKMCLFAGFQRKAVVVCPKDEDYKQRTQKKAEVEGKDLPEHAVLKMKGNFTLPEVAECFDEITYVELQKEEAQKLLEQYKEESKKALPPEKKQNTGSKKSNKNKSGKNQFNRGGGHRGRGGLNMRGGNFRGGAPGNRGGYNRRGNMPQRGGGGGGSGGIGYPYPRAPVFPGRGSYSNRGNYNRGGMPNRGNYNQNFRGRGNNRGYKNQSQGYNQWQQGQFWGQKPWSQHYHQGYY

SEQ ID NO: 24 - protein, 1178 aa, Homo sapiens
MLKFRTVHGGLRLLGIRRTSTAPAASPNVRRLEYKPIKKVMVANRGEIAIRVFRACTELGIRTVAIYSEQDTGQMHRQKADEAYLIGRGLAPVQAYLHIPDIIKVAKENNVDAVHPGYGFLSERADFAQACQDAGVRFIGPSPEVVRKMGDKVEARAIAIAAGVPVVPGTDAPITSLHEAHEFSNTYGFPIIFKAAYGGGGRGMRVVHSYEELEENYTRAYSEALAAFGNGALFVEKFIEKPRHIEVQILGDQYGNILHLYERDCSIQRRHQKVVEIAPAAHLDPQLRTRLTSDSVKLAKQVGYENAGTVEFLVDRHGKHYFIEVNSRLQVEHTVTEEITDVDLVHAQIHVSEGRSLPDLGLRQENIRINGCAIQCRVTTEDPARSFQPDTGRIEVFRSGEGMGIRLDNASAFQGAVISPHYDSLLVKVIAHGKDHPTAATKMSRALAEFRVRGVKTNIAFLQNVLNNQQFLAGTVDTQFIDENPELFQLRPAQNRAQKLLHYLGHVMVNGPTTPIPVKASPSPTDPVVPAVPIGPPPAGFRDILLREGPEGFARAVRNHPGLLLMDTTFRDAHQSLLATRVRTHDLKKIAPYVAHNFSKLFSMENWGGATFDVAMRFLYECPWRRLQELRELIPNIPFQMLLRGANAVGYTNYPDNVVFKFCEVAKENGMDVFRVFDSLNYLPNMLLGMEAAGSAGGVVEAAISYTGDVADPSRTKYSLQYYMGLAEELVRAGTHILCIKDMAGLLKPTACTMLVSSLRDRFPDLPLHIHTHDTSGAGVAAMLACAQAGADVVDVAADSMSGMTSQPSMGALVACTRGTPLDTEVPMERVFDYSEYWEGARGLYAAFDCTATMKSGNSDVYENEIPGGQYTNLHFQAHSMGLGSKFKEVKKAYVEANQMLGDLIKVTPSSKIVGDLAQFMVQNGLSRAEAEAQAEELSFPRSVVEFLQGYIGVPHGGFPEPFRSKVLKDLPRVEGRPGASLPPLDLQALEKELVDRHGEEVTPEDVLSAAMYPDVFAHFKDFTATFGPLDSLNTRLFLQGPKIAEEFEVELERGKTLHIKALAVSDLNRAGQRQVFFELNGQLRSILVKDTQAMKEMHFHPKALKDVKGQIGAPMPGKVIDIKVVAGAKVAKGQPLCVLSAMKMETVVTSPMEGTVRKVHVTKDMTLEGDDLILEIE

SEQ ID NO: 25 - protein, 1178 aa, Homo sapiens; identical to SEQ ID NO: 24

SEQ ID NO: 26 - protein, 744 aa, Homo sapiens
MELCGATRLGYFGRSQFYIALKLVAVAQSGFPLRVESINTVKDLPLPRFVASKNEQESRHAASYSSDSENQGSYSGVIPPPPGRGQVKKGSVSHDTVQPRTSADAQEPASPVVSPQQSPPTSPHTWRKHSRHPSGGNSERPLAGPGPFWSPFGEAQSGSSAGDAVWSGHSPPPPQENWVSFADTPPTSTLLTMHPASVQDQTTVRTVASATTAIEIRRQSSSYDDPWKITDEQRQYYVNQFKTIQPDLNGFIPGSAAKEFFTKSKLPILELSHIWELSDFDKDGALTLDEFCAAFHLVVARKNGYDLPEKLPESLMPKLIDLEDSADVGDQPGEVGYSGSPAEAPPSKSPSMPSLNQTWPELNQSSEQWETFSERSSSSQTLTQFDSNIAPADPDTAIVHPVPIRMTPSKIHMQEMELKRTGSDHTNPTSPLLVKPSDLLEENKINSSVKFASGNTVADGYSSSDSFTSDPEQIGSNVTRQRSHSGTSPDNTAPPPPPPRPQPSHSRSSSLDMNRTFTVTTGQQQAGVVAHPPAVPPRPQPSQAPGPAVHRPVDADGLITHTSTSPQQIPEQPNFVDFSQFEVFAASNVNDEQDDEAEKHPEVLPAEKASDPASSLRVAKTDSKTEEKTAASAPANVSKGTTPLAPPPKPVRRRLKSEDELRPEVDEHTQKTGVLAAVLASQPSIPRSVGKDKKAIQASIRRNKETNTVLARLNSELQQQLKDVLEERISLEVQLEQLRPFSHL

SEQ ID NO: 27 - protein, 1960 aa, Homo sapiens
MAQQAADKYLYVDKNFINNPLAQADWAAKKLVWVPSDKSGFEPASLKEEVGEEAIVELVENGKKVKVNKDDIQKMNPPKFSKVEDMAELTCLNEASVLHNLKERYYSGLIYTYSGLFCVVINPYKNLPIYSEEIVEMYKGKKRHEMPPHIYAITDTAYRSMMQDREDQSILCTGESGAGKTENTKKVIQYLAYVASSHKSKKDQGELERQLLQANPILEAFGNAKTVKNDNSSRFGKFIRINFDVNGYIVGANIETYLLEKSRAIRQAKEERTFHIFYYLLSGAGEHLKTDLLLEPYNKYRFLSNGHVTIPGQQDKDMFQETMEAMRIMGIPEEEQMGLLRVISGVLQLGNIVFKKERNTDQASMPDNTAAQKVSHLLGINVTDFTRGILTPRIKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVMQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNSIPKGFMDGKQACVLMIKALELDSNLYRIGQSKVFFRAGVLAHLEEERDLKITDVIIGFQACCRGYLARKAFAKRQQQLTAMKVLQRNCAAYLKLRNWQWWRLFTKVKPLLQVSRQEEEMMAKEEELVKVREKQLAAENRLTEMETLQSQLMAEKLQLQEQLQAETELCAEAEELRARLTAKKQELEEICHDLEARVEEEEERCQHLQAEKKKMQQNIQELEEQLEEEESARQKLQLEKVTTEAKLKKLEEEQIILEDQNCKLAKEKKLLEDRIAEFTTNLTEEEEKSKSLAKLKNKHEAMITDLEERLRREEKQRQELEKTRRKLEGDSTDLSDQIAELQAQIAELKMQLAKKEEELQAALARVEEEAAQKNMALKKIRELESQISELQEDLESERASRNKAEKQKRDLGEELEALKTELEDTLDSTAAQQELRSKREQEVNILKKTLEEEAKTHEAQIQEMRQKHSQAVEELAEQLEQTKRVKANLEKAKQTLENERGELANEVKVLLQGKGDSEHKRKKVEAQLQELQVKFNEGERVRTELADKVTKLQVELDNVTGLLSQSDSKSSKLTKDFSALESQLQDTQELLQEENRQKLSLSTKLKQVEDEKNSFREQLEEEEEAKHNLEKQIATLHAQVADMKKKMEDSVGCLETAEEVKRKLQKDLEGLSQRHEEKVAAYDKLEKTKTRLQQELDDLLVDLDHQRQSACNLEKKQKKFDQLLAEEKTISAKYAEERDRAEAEAREKETKALSLARALEEAMEQKAELERLNKQFRTEMEDLMSSKDDVGKSVHELEKSKRALEQQVEEMKTQLEELEDELQATEDAKLRLEVNLQAMKAQFERDLQGRDEQSEEKKKQLVRQVREMEAELEDERKQRSMAVAARKKLEMDLKDLEAHIDSANKNRDEAIKQLRKLQAQMKDCMRELDDTRASREEILAQAKENEKKLKSMEAEMIQLQEELAAAERAKRQAQQERDELADEIANSSGKGALALEEKRRLEARIAQLEEELEEEQGNTELINDRLKKANLQIDQINTDLNLERSHAQKNENARQQLERQNKELKVKLQEMEGTVKSKYKASITALEAKIAQLEEQLDNETKERQAACKQVRRTEKKLKDVLLQVDDERRNAEQYKDQADKASTRLKQLKRQLEEAEEEAQRANASRRKLQRELEDATETADAMNREVSSLKNKLRRGDLPFVVPRRMARKGAGDGSDEEVDGKADGAEAKPAE

SEQ ID NO: 28 - protein, 679 aa, Homo sapiens
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 29 - protein, 9 aa, Homo sapiens
VTEPGTAQY

SEQ ID NO: 30 - DNA, 21 bp, Artificial Sequence
  Description of Combined DNA/RNA Molecule Synthetic siRNA oligonucleotide
cuggaagucaagcuccucctt

SEQ ID NO: 31 - DNA, 21 bp, Artificial Sequence
  Description of Combined DNA/RNA Molecule Synthetic siRNA oligonucleotide
ccagucgcaaacgcgacugtt